(19) United States
(12) Patent Application Publication
(10) Pub. No.: US 2025/0179533 A1
(43) Pub. Date: Jun. 5, 2025

(54) RETROVIRAL-LIKE PARTICLE-REDUCED CHINESE HAMSTER OVARY PRODUCTION CELL LINES

(71) Applicant: GENENTECH, INC., South San Francisco, CA (US)

(72) Inventors: Zion Lee, South San Francisco, CA (US); Amy Shen, South San Francisco, CA (US); Gavin Christian Barnard, South San Francisco, CA (US); Qi Chen, South San Francisco, CA (US); Hua Wang, South San Francisco, CA (US); Bin Yang, South San Francisco, CA (US); Min Zhang, South San Francisco, CA (US)

(73) Assignee: GENENTECH, INC., South San Francisco, CA (US)

(21) Appl. No.: 18/919,812

(22) Filed: Oct. 18, 2024

Related U.S. Application Data
(60) Provisional application No. 63/544,782, filed on Oct. 18, 2023.

(51) Int. Cl. C12N 15/90; C07K 16/18; C07K 16/22; C07K 16/24; C07K 16/28; C07K 16/32; C07K 16/40; C07K 16/42; C12N 9/22; C12N 15/11

(52) U.S. Cl. CPC C12N 15/907; C07K 16/18; C07K 16/22; C07K 16/244; C07K 16/249; C07K 16/2803; C07K 16/2809; C07K 16/2812; C07K 16/2827; C07K 16/2839; C07K 16/2845; C07K 16/2863; C07K 16/2875; C07K 16/2878; C07K 16/2887; C07K 16/32; C07K 16/40; C07K 16/4291; C12N 9/22; C12N 15/11; C12N 2310/20

(57) ABSTRACT

The presently disclosed subject matter relates to retroviral-like particle (RVLP)-reduced Chinese hamster ovary (CHO) cells suitable to produce recombinant proteins, as well as methods of producing and using such RVLP-reduced CHO production cells.

### CROSS-REFERENCE TO RELATED APPLICATIONS

This application claims priority to U.S. Provisional Application No. 63/544,782, filed Oct. 18, 2023, the contents of which are incorporated by reference herein in their entireties and to which priority is claimed.

### SEQUENCE LISTING

The present specification makes reference to a Sequence Listing (submitted electronically as a xml file named “00B206_1462_SL.xml” on Oct. 18, 2024). The 00B206_1462_SL.xml file was generated on Oct. 17, 2024, and is 6,639,945 bytes in size. The entire contents of the Sequence Listing are hereby incorporated by reference.

### TECHNICAL FIELD

The presently disclosed subject matter relates to retroviral-like particle (RVLP)-reduced Chinese hamster ovary (CHO) cells suitable to produce recombinant proteins, as well as methods of producing and using such RVLP-reduced CHO production cells.

### BACKGROUND

Due to the rapid advancement in cell biology and immunology, there has been an increasing demand to develop novel therapeutic recombinant proteins for a variety of diseases including cancer, cardiovascular diseases and metabolic diseases. These biopharmaceutical candidates are commonly manufactured by commercial cell lines capable of expressing the proteins of interest. For example, CHO cells have been widely adopted for the production of monoclonal antibodies.

That CHO cells are a preferred mammalian expression system for biomanufacturing is due, in part, to their safety profile relative to other mammalian expression systems. Not only are CHO cells less susceptible to certain viral infections relative to other mammalian cells, but studies have repeatedly found CHO cells unable to produce infective viral-like particles (VLPs), e.g., infective RVLPs, capable of replicating in human cells. CHO cells do, however, produce VLPs and such VLPs have been identified both intracellularly and extracellularly in cell culture media. Because such VLPs, e.g., RVLPs, are believed to result from the presence of endogenous retroviruses (ERVs), as opposed to newly acquired retroviral infections, there remains a need in the art for RVLP-reduced CHO cells suitable to facilitate the commercial production of recombinant proteins.

### SUMMARY OF THE INVENTION

The presently disclosed subject matter relates, in certain embodiments, to modified CHO cells where, prior to the modification, the CHO cells comprise two or more ERV loci selected from:
(a) CHERV-1b (Genbank Accession No. MN527960, SEQ ID NO. 8);
(b) CHERV-2g (Genbank Accession No. MN527961, SEQ ID NO. 9);
(c) ETC109F (30021-39247 of SEQ ID NO. 10);
(d) CHERV-3g (Genbank Accession No. MN527962, SEQ ID NO. 11); and
(e) an ERV locus comprising a sequence having at least 90% identity to any one of (a)-(d), where the modification comprises inactivation of two or more of the ERV loci of (a)-(e) present in the CHO cell prior to such modification. In certain embodiments, inactivation of the two or more of the ERV loci of (a)-(e) comprises a knock-out of the respective ERV GAG coding sequence. For example, but not by way of limitation, the knock-out of the respective ERV GAG coding sequence can comprise introduction of an indel into the ERV GAG coding sequence. In certain non-limiting embodiments, the knock-out of the respective ERV GAG coding sequence can comprise introduction of a base edit into the ERV GAG coding sequence. In certain non-limiting embodiments, the knock-out of the respective ERV GAG coding sequence can comprise introduction of a deletion flanking the ERV GAG coding sequence.

In certain embodiments, the presently disclosed subject matter is directed to modified CHO cells where, prior to the modification, the CHO cells comprise two or more ERV loci selected from:
(a) CHERV-1b (Genbank Accession No. MN527960, SEQ ID NO. 8);
(b) CHERV-2g (Genbank Accession No. MN527961, SEQ ID NO. 9);
(c) ETC109F (30021-39247 of SEQ ID NO. SEQ ID NO. 10);
(d) CHERV-3g (Genbank Accession No. MN527962, SEQ ID NO. 11); and
(e) an ERV locus comprising a sequence having at least 90% identity to any one of (a)-(d), where the modification comprises inactivation of two or more of the ERV loci of (a)-(e) present in the CHO cell prior to such modification, and wherein the modified cell expresses a recombinant product of interest. In certain embodiments, the modified cell is generated from a recombinant cell that expresses a recombinant product of interest. In certain embodiments, the recombinant product of interest comprises a recombinant protein. In certain embodiments, the recombinant protein is antibody or an antigen-binding fragment thereof. In certain embodiments, the antibody is a multispecific antibody or an antigen-binding fragment thereof. In certain embodiments, the antibody consists of a single heavy chain sequence and a single light chain sequence or antigen-binding fragments thereof. In certain embodiments, the antibody is a chimeric antibody, a human antibody or a humanized antibody. In certain embodiments, the antibody is a monoclonal antibody.

In certain embodiments, the recombinant product of interest expressed by a modified CHO cell of the present disclosure is encoded by an exogenous nucleic acid sequence integrated at one or more targeted locations in the cellular genome of the modified CHO cell. In certain embodiments, the targeted location is at least about 90% homologous to a sequence of a portion of the contig sequence of one of the contigs NW_006874047.1, NW_006884592.1, NW_006881296.1, NW_003616412.1, NW_003615063.1, NW_006882936.1, and NW_003615411.1, or to a sequence selected from SEQ ID Nos. 1-7.

In certain embodiments, the modified CHO cell of the present disclosure comprises gene knock-outs selected from: a) BAX; BAK; ICAM-1; GGTA1; CMAH; LPL; LPLA2; and PPT1; b) BAX; BAK; ICAM-1; SIRT-1; GGTA1; CMAH; LPL, LPLA2; and PPT1; c) BAX; BAK; ICAM-1; SIRT-1; MYC; GGTA1; CMAH; LPL; LPLA2; and PPT1; d) BAX; BAK; ICAM-1; SIRT-1; MYC; GGTA1; CMAH; LPL, LPLA2; PPT1; and LIPA; e) BAX; BAK; ICAM-1; GGTA1; CMAH; LPLA2; PPT1; and LIPA; f) BAX; BAK; ICAM-1; SIRT-1; GGTA1; CMAH; LPLA2; PPT1; and LIPA; g) BAX; BAK; ICAM-1; SIRT-1; MYC; GGTA1; CMAH; LPLA2; PPT1; and LIPA; h) BAX; BAK; ICAM-1; LPL; LPLA2; and PPT1; i) BAX; BAK; ICAM-1; SIRT-1; LPL, LPLA2; and PPT1; j) BAX; BAK; ICAM-1; SIRT-1; MYC; LPL; LPLA2; and PPT1; k) BAX; BAK; ICAM-1; SIRT-1; MYC; LPL; LPLA2; PPT1; and LIPA; 1) BAX; BAK; ICAM-1; SIRT-1; and MYC; m) BAX; BAK; ICAM-1; PERK; SIRT-1; and MYC; n) BAX; BAK; ICAM-1; SIRT-1; PERK; MYC; GGTA1; CMAH; LPL, LPLA2; PPT1; and LIPA; o) BAX; BAK; LPL; LPLA2; GGTA1; and CMAH; p) BAX; BAK; PERK; LPL; LPLA2; GGTA1; and CMAH; q) BAX; BAK; MYC; LPL; LPLA2; GGTA1; and CMAH; r) BAX; BAK; MYC; PERK; LPL; LPLA2; GGTA1; and CMAH; s) BAX; BAK; LPL; LPLA2; GGTA1; CMAH; and PPT1; t) BAX; BAK; PERK; LPL; LPLA2; GGTA1; CMAH; and PPT1; u) BAX; BAK; MYC; LPL; LPLA2; GGTA1; CMAH; and PPT1; v) BAX; BAK; MYC; PERK; LPL; LPLA2; GGTA1; CMAH; and PPT1; w) BAX; BAK; ICAM-1; and SIRT-1; x) BAX; BAK; and ICAM-1; y) BAX; BAK; BCKDHA; ICAM-1; GGTA1; CMAH; LPL; LPLA2; and PPT1; z) BAX; BAK; BCKDHA; ICAM-1; SIRT-1; GGTA1; CMAH; LPL; LPLA2; and PPT1; aa) BAX; BAK; BCKDHA; ICAM-1; SIRT-1; MYC; GGTA1; CMAH; LPL; LPLA2; and PPT1; bb) BAX; BAK; BCKDHA; ICAM-1; SIRT-1; MYC; GGTA1; CMAH; LPL, LPLA2; PPT1; and LIPA; cc) BAX; BAK; BCKDHA; ICAM-1; GGTA1; CMAH; LPLA2; PPT1; and LIPA; dd) BAX; BAK; BCKDHA; ICAM-1; SIRT-1; GGTA1; CMAH; LPLA2; PPT1; and LIPA; ee) BAX; BAK; BCKDHA; ICAM-1; SIRT-1; MYC; GGTA1; CMAH; LPLA2; PPT1; and LIPA; ff) BAX; BAK; BCKDHA; ICAM-1; LPL; LPLA2; and PPT1; gg) BAX; BAK; BCKDHA; ICAM-1; SIRT-1; LPL, LPLA2; and PPT1; hh) BAX; BAK; BCKDHA; ICAM-1; SIRT-1; MYC; LPL; LPLA2; and PPT1; ii) BAX; BAK; BCKDHA; ICAM-1; SIRT-1; MYC; LPL; LPLA2; PPT1; and LIPA; jj) BAX; BAK; BCKDHA; ICAM-1; SIRT-1; and MYC; kk) BAX; BAK; BCKDHA; ICAM-1; PERK; SIRT-1; and MYC; 11) BAX; BAK; BCKDHA; ICAM-1; PERK; SIRT-1; MYC; GGTA1; CMAH; LPL, LPLA2; PPT1; and LIPA; mm) BAX; BAK; BCKDHA; LPL; LPLA2; GGTA1; and CMAH; nn) BAX; BAK; BCKDHA; PERK; LPL; LPLA2; GGTA1; and CMAH; oo) BAX; BAK; BCKDHA; MYC; LPL; LPLA2; GGTA1; and CMAH; pp) BAX; BAK; BCKDHA; MYC; PERK; LPL; LPLA2; GGTA1; and CMAH; qq) BAX; BAK; BCKDHA; LPL; LPLA2; GGTA1; CMAH; and PPT1; rr) BAX; BAK; BCKDHA; PERK; LPL; LPLA2; GGTA1; CMAH; and PPT1; ss) BAX; BAK; BCKDHA; MYC; LPL; LPLA2; GGTA1; CMAH; and PPT1; tt) BAX; BAK; BCKDHA; MYC; PERK; LPL; LPLA2; GGTA1; CMAH; and PPT1; uu) BAX; BAK; BCKDHA; ICAM-1; and SIRT-1; vv) BAX; BAK; BCKDHA; and ICAM-1; ww) BAX; BAK; BCKDHB; ICAM-1; GGTA1; CMAH; LPL; LPLA2; and PPT1; xx) BAX; BAK; BCKDHB; ICAM-1; SIRT-1; GGTA1; CMAH; LPL; LPLA2; and PPT1; yy) BAX; BAK; BCKDHB; ICAM-1; SIRT-1; MYC; GGTA1; CMAH; LPL; LPLA2; and PPT1; zz) BAX; BAK; BCKDHB; ICAM-1; SIRT-1; MYC; GGTA1; CMAH; LPL, LPLA2; PPT1; and LIPA; aaa) BAX; BAK; BCKDHB; ICAM-1; GGTA1; CMAH; LPLA2; PPT1; and LIPA; bbb) BAX; BAK; BCKDHB; ICAM-1; SIRT-1; GGTA1; CMAH; LPLA2; PPT1; and LIPA; ccc) BAX; BAK; BCKDHB; ICAM-1; SIRT-1; MYC; GGTA1; CMAH; LPLA2; PPT1; and LIPA; ddd) BAX; BAK; BCKDHB; ICAM-1; LPL; LPLA2; and PPT1; eee) BAX; BAK; BCKDHB; ICAM-1; SIRT-1; LPL, LPLA2; and PPT1; fff) BAX; BAK; BCKDHB; ICAM-1; SIRT-1; MYC; LPL; LPLA2; and PPT1; ggg) BAX; BAK; BCKDHB; ICAM-1; SIRT-1; MYC; LPL; LPLA2; PPT1; and LIPA; hhh) BAX; BAK; BCKDHB; ICAM-1; SIRT-1; and MYC; iii) BAX; BAK; BCKDHB; ICAM-1; PERK; SIRT-1; and MYC; jjj) BAX; BAK; BCKDHB; ICAM-1; PERK; SIRT-1; MYC; GGTA1; CMAH; LPL, LPLA2; PPT1; and LIPA; kkk) BAX; BAK; BCKDHB; LPL; LPLA2; GGTA1; and CMAH; 111) BAX; BAK; BCKDHB; PERK; LPL; LPLA2; GGTA1; and CMAH; mmm) BAX; BAK; BCKDHB; MYC; LPL; LPLA2; GGTA1; and CMAH; nnn) BAX; BAK; BCKDHB; MYC; PERK; LPL; LPLA2; GGTA1; and CMAH; ooo) BAX; BAK; BCKDHB; LPL; LPLA2; GGTA1; CMAH; and PPT1; ppp) BAX; BAK; BCKDHB; PERK; LPL; LPLA2; GGTA1; CMAH; and PPT1; qqq) BAX; BAK; BCKDHB; MYC; LPL; LPLA2; GGTA1; CMAH; and PPT1; rrr) BAX; BAK; BCKDHB; MYC; PERK; LPL; LPLA2; GGTA1; CMAH; and PPT1; sss) BAX; BAK; BCKDHB; ICAM-1; and SIRT-1; ttt) BAX; BAK; BCKDHB; and ICAM-1; uuu) BAX; BAK; BCKDHA; BCKDHB; ICAM-1; GGTA1; CMAH; LPL; LPLA2; and PPT1; vvv) BAX; BAK; BCKDHA; BCKDHB; ICAM-1; SIRT-1; GGTA1; CMAH; LPL; LPLA2; and PPT1; www) BAX; BAK; BCKDHA; BCKDHB; ICAM-1; SIRT-1; MYC; GGTA1; CMAH; LPL; LPLA2; and PPT1; xxx) BAX; BAK; BCKDHA; BCKDHB; ICAM-1; SIRT-1; MYC; GGTA1; CMAH; LPL, LPLA2; PPT1; and LIPA; yyy) BAX; BAK; BCKDHA; BCKDHB; ICAM-1; GGTA1; CMAH; LPLA2; PPT1; and LIPA; zzz) BAX; BAK; BCKDHA; BCKDHB; ICAM-1; SIRT-1; GGTA1; CMAH; LPLA2; PPT1; and LIPA; aaaa) BAX; BAK; BCKDHA; BCKDHB; ICAM-1; SIRT-1; MYC; GGTA1; CMAH; LPLA2; PPT1; and LIPA; bbbb) BAX; BAK; BCKDHA; BCKDHB; ICAM-1; LPL; LPLA2; and PPT1; cccc) BAX; BAK; BCKDHA; BCKDHB; ICAM-1; SIRT-1; LPL, LPLA2; and PPT1; dddd) BAX; BAK; BCKDHA; BCKDHB; ICAM-1; SIRT-1; MYC; LPL; LPLA2; and PPT1; eeee) BAX; BAK; BCKDHA; BCKDHB; ICAM-1; SIRT-1; MYC; LPL; LPLA2; PPT1; and LIPA; ffff) BAX; BAK; BCKDHA; BCKDHB; ICAM-1; SIRT-1; and MYC; gggg) BAX; BAK; BCKDHA; BCKDHB; ICAM-1; PERK; SIRT-1; and MYC; hhhh) BAX; BAK; BCKDHA; BCKDHB; ICAM-1; PERK; SIRT-1; MYC; GGTA1; CMAH; LPL, LPLA2; PPT1; and LIPA; iiii) BAX; BAK; BCKDHA; BCKDHB; LPL; LPLA2; GGTA1; and CMAH; jjjj) BAX; BAK; BCKDHA; BCKDHB; PERK; LPL; LPLA2; GGTA1; and CMAH; kkkk) BAX; BAK; BCKDHA; BCKDHB; MYC; LPL; LPLA2; GGTA1; and CMAH; llll) BAX; BAK; BCKDHA; BCKDHB; MYC; PERK; LPL; LPLA2; GGTA1; and CMAH; mmmm) BAX; BAK; BCKDHA; BCKDHB; LPL; LPLA2; GGTA1; CMAH; and PPT1; nnnn) BAX; BAK; BCKDHA; BCKDHB; PERK; LPL; LPLA2; GGTA1; CMAH; and PPT1; oooo) BAX; BAK; BCKDHA; BCKDHB; MYC; LPL; LPLA2; GGTA1; CMAH; and PPT1; pppp) BAX; BAK; BCKDHA; BCKDHB; MYC; PERK; LPL; LPLA2; GGTA1; CMAH; and PPT1; qqqq) BAX; BAK; BCKDHA; BCKDHB; ICAM-1; and SIRT-1; or rrrr) BAX; BAK; BCKDHA; BCKDHB; and ICAM-1.

In certain embodiments, the present disclosure is directed to compositions comprising a modified CHO cell as disclosed herein.

In certain embodiments, the present disclosure is directed to methods of producing a recombinant product of interest comprising:
a) culturing a modified CHO cell of the present disclosure; and
b) recovering the recombinant product of interest from the cultivation medium or the modified CHO cells.

In certain embodiments, the methods of producing a recombinant product of interest of the present disclosure comprise purifying the recombinant product of interest, harvesting the recombinant product of interest, and/or formulating the recombinant product of interest.

In certain embodiments, the present disclosure is directed to methods of producing a modified CHO cell, comprising:
(a) contacting the cell with a nuclease-assisted gene targeting system and/or nucleic acid targeting at least two endogenous ERVs selected from
   (i) CHERV-1b (Genbank Accession No. MN527960, SEQ ID NO. 8);
   (ii) CHERV-2g (Genbank Accession No. MN527961, SEQ ID NO. 9);
   (iii) ETC109F (30021-39247 of SEQ ID NO. 10);
   (iv) CHERV-3g (Genbank Accession No. MN527962, SEQ ID NO. 11); and
   (v) an ERV locus comprising a sequence having at least 90% identity to any one of (i)-(iv), and
   (b) selecting the modified CHO cell wherein the expression of said ERVs has been reduced or eliminated as compared to an unmodified CHO cell.

In certain embodiments, the methods of producing a modified CHO cell of the present disclosure comprise introducing an exogenous nucleic acid encoding a recombinant product of interest is into the CHO cell after the modification where the expression of said ERVs is reduced or eliminated as compared to an unmodified CHO cell. In certain embodiments, the methods of producing a modified CHO cell of the present disclosure comprise introducing an exogenous nucleic acid encoding a recombinant product of interest is into the CHO cell prior to the modification where the expression of said ERVs is reduced or eliminated as compared to an unmodified CHO cell.

In certain embodiments, the exogenous nucleic acid encoding a recombinant product of interest introduced into the modified cell of the present disclosure, either prior to or after said modification, is integrated in the cellular genome of the CHO cell at one or more targeted locations. In certain embodiments, the targeted location is at least about 90% homologous to a sequence of a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7.

In certain embodiments, the exogenous nucleic acid encoding a recombinant product of interest introduced into the modified cell of the present disclosure, either prior to or after said modification, is randomly integrated in the cellular genome of the modified CHO cells.

In certain embodiments, the recombinant product of interest encoded by the exogenous nucleic acid encoding introduced into the modified cell of the present disclosure, either prior to or after said modification, comprises a recombinant protein. In certain embodiments, the recombinant protein is an antibody or an antigen-binding fragment thereof. In certain embodiments, antibody is a multispecific antibody or an antigen-binding fragment thereof. In certain embodiments, the antibody consists of a single heavy chain sequence and a single light chain sequence or antigen-binding fragments thereof. In certain embodiments, the antibody is a chimeric antibody, a human antibody or a humanized antibody. In certain embodiments, the antibody is a monoclonal antibody.

In certain embodiments, the nucleic acid sequence encoding the recombinant product of interest introduced into the modified cell of the present disclosure, either prior to or after said modification, is introduced using a transposase-mediated gene integration system.

In certain embodiments, the nuclease-assisted gene targeting system employed for the reduction of ERV expression in the modified CHO cells of the present disclosure is selected from the group consisting of CRISPR/Cas9, CRISPR/Cpf1, zinc-finger nuclease, TALEN or meganuclease.

In certain embodiments, the reduction of ERV expression in the modified CHO cells of the present disclosure is mediated by RNA silencing. In certain embodiments, the RNA silencing is selected from the group consisting of siRNA gene targeting and knock-down, shRNA gene targeting and knock-down, and miRNA gene targeting and knock-down.

In certain embodiments, the methods of producing a modified CHO cell of the present disclosure comprise use of a CHO cells comprising gene knock-outs selected from: a) BAX; BAK; ICAM-1; GGTA1; CMAH; LPL; LPLA2; and PPT1; b) BAX; BAK; ICAM-1; SIRT-1; GGTA1; CMAH; LPL, LPLA2; and PPT1; c) BAX; BAK; ICAM-1; SIRT-1; MYC; GGTA1; CMAH; LPL; LPLA2; and PPT1; d) BAX; BAK; ICAM-1; SIRT-1; MYC; GGTA1; CMAH; LPL, LPLA2; PPT1; and LIPA; e) BAX; BAK; ICAM-1; GGTA1; CMAH; LPLA2; PPT1; and LIPA; f) BAX; BAK; ICAM-1; SIRT-1; GGTA1; CMAH; LPLA2; PPT1; and LIPA; g) BAX; BAK; ICAM-1; SIRT-1; MYC; GGTA1; CMAH; LPLA2; PPT1; and LIPA; h) BAX; BAK; ICAM-1; LPL; LPLA2; and PPT1; i) BAX; BAK; ICAM-1; SIRT-1; LPL, LPLA2; and PPT1; j) BAX; BAK; ICAM-1; SIRT-1; MYC; LPL; LPLA2; and PPT1; k) BAX; BAK; ICAM-1; SIRT-1; MYC; LPL; LPLA2; PPT1; and LIPA; 1) BAX; BAK; ICAM-1; SIRT-1; and MYC; m) BAX; BAK; ICAM-1; PERK; SIRT-1; and MYC; n) BAX; BAK; ICAM-1; SIRT-1; PERK; MYC; GGTA1; CMAH; LPL, LPLA2; PPT1; and LIPA; o) BAX; BAK; LPL; LPLA2; GGTA1; and CMAH; p) BAX; BAK; PERK; LPL; LPLA2; GGTA1; and CMAH; q) BAX; BAK; MYC; LPL; LPLA2; GGTA1; and CMAH; r) BAX; BAK; MYC; PERK; LPL; LPLA2; GGTA1; and CMAH; s) BAX; BAK; LPL; LPLA2; GGTA1; CMAH; and PPT1; t) BAX; BAK; PERK; LPL; LPLA2; GGTA1; CMAH; and PPT1; u) BAX; BAK; MYC; LPL; LPLA2; GGTA1; CMAH; and PPT1; v) BAX; BAK; MYC; PERK; LPL; LPLA2; GGTA1; CMAH; and PPT1; w) BAX; BAK; ICAM-1; and SIRT-1; x) BAX; BAK; and ICAM-1; y) BAX; BAK; BCKDHA; ICAM-1; GGTA1; CMAH; LPL; LPLA2; and PPT1; z) BAX; BAK; BCKDHA; ICAM-1; SIRT-1; GGTA1; CMAH; LPL; LPLA2; and PPT1; aa) BAX; BAK; BCKDHA; ICAM-1; SIRT-1; MYC; GGTA1; CMAH; LPL; LPLA2; and PPT1; bb) BAX; BAK; BCKDHA; ICAM-1; SIRT-1; MYC; GGTA1; CMAH; LPL, LPLA2; PPT1; and LIPA; cc) BAX; BAK; BCKDHA; ICAM-1; GGTA1; CMAH; LPLA2; PPT1; and LIPA; dd) BAX; BAK; BCKDHA; ICAM-1; SIRT-1; GGTA1; CMAH; LPLA2; PPT1; and LIPA; ee) BAX; BAK; BCKDHA; ICAM-1; SIRT-1; MYC; GGTA1; CMAH; LPLA2; PPT1; and LIPA; ff) BAX; BAK; BCKDHA; ICAM-1; LPL; LPLA2; and PPT1; gg) BAX; BAK; BCKDHA; ICAM-1; SIRT-1; LPL, LPLA2; and PPT1; hh) BAX; BAK; BCKDHA; ICAM-1; SIRT-1; MYC; LPL; LPLA2; and PPT1; ii) BAX; BAK; BCKDHA; ICAM-1; SIRT-1; MYC; LPL; LPLA2; PPT1; and LIPA; jj) BAX; BAK; BCKDHA; ICAM-1; SIRT-1; and MYC; kk) BAX; BAK; BCKDHA; ICAM-1; PERK; SIRT-1; and MYC; 11) BAX; BAK; BCKDHA; ICAM-1; PERK; SIRT-1; MYC; GGTA1; CMAH; LPL, LPLA2; PPT1; and LIPA; mm) BAX; BAK; BCKDHA; LPL; LPLA2; GGTA1; and CMAH; nn) BAX; BAK; BCKDHA; PERK; LPL; LPLA2; GGTA1; and CMAH; oo) BAX; BAK; BCKDHA; MYC; LPL; LPLA2; GGTA1; and CMAH; pp) BAX; BAK; BCKDHA; MYC; PERK; LPL; LPLA2; GGTA1; and CMAH; qq) BAX; BAK; BCKDHA; LPL; LPLA2; GGTA1; CMAH; and PPT1; rr) BAX; BAK; BCKDHA; PERK; LPL; LPLA2; GGTA1; CMAH; and PPT1; ss) BAX; BAK; BCKDHA; MYC; LPL; LPLA2; GGTA1; CMAH; and PPT1; tt) BAX; BAK; BCKDHA; MYC; PERK; LPL; LPLA2; GGTA1; CMAH; and PPT1; uu) BAX; BAK; BCKDHA; ICAM-1; and SIRT-1; vv) BAX; BAK; BCKDHA; and ICAM-1; ww) BAX; BAK; BCKDHB; ICAM-1; GGTA1; CMAH; LPL; LPLA2; and PPT1; xx) BAX; BAK; BCKDHB; ICAM-1; SIRT-1; GGTA1; CMAH; LPL; LPLA2; and PPT1; yy) BAX; BAK; BCKDHB; ICAM-1; SIRT-1; MYC; GGTA1; CMAH; LPL; LPLA2; and PPT1; zz) BAX; BAK; BCKDHB; ICAM-1; SIRT-1; MYC; GGTA1; CMAH; LPL, LPLA2; PPT1; and LIPA; aaa) BAX; BAK; BCKDHB; ICAM-1; GGTA1; CMAH; LPLA2; PPT1; and LIPA; bbb) BAX; BAK; BCKDHB; ICAM-1; SIRT-1; GGTA1; CMAH; LPLA2; PPT1; and LIPA; ccc) BAX; BAK; BCKDHB; ICAM-1; SIRT-1; MYC; GGTA1; CMAH; LPLA2; PPT1; and LIPA; ddd) BAX; BAK; BCKDHB; ICAM-1; LPL; LPLA2; and PPT1; eee) BAX; BAK; BCKDHB; ICAM-1; SIRT-1; LPL, LPLA2; and PPT1; fff) BAX; BAK; BCKDHB; ICAM-1; SIRT-1; MYC; LPL; LPLA2; and PPT1; ggg) BAX; BAK; BCKDHB; ICAM-1; SIRT-1; MYC; LPL; LPLA2; PPT1; and LIPA; hhh) BAX; BAK; BCKDHB; ICAM-1; SIRT-1; and MYC; iii) BAX; BAK; BCKDHB; ICAM-1; PERK; SIRT-1; and MYC; jjj) BAX; BAK; BCKDHB; ICAM-1; PERK; SIRT-1; MYC; GGTA1; CMAH; LPL, LPLA2; PPT1; and LIPA; kkk) BAX; BAK; BCKDHB; LPL; LPLA2; GGTA1; and CMAH; 111) BAX; BAK; BCKDHB; PERK; LPL; LPLA2; GGTA1; and CMAH; mmm) BAX; BAK; BCKDHB; MYC; LPL; LPLA2; GGTA1; and CMAH; nnn) BAX; BAK; BCKDHB; MYC; PERK; LPL; LPLA2; GGTA1; and CMAH; ooo) BAX; BAK; BCKDHB; LPL; LPLA2; GGTA1; CMAH; and PPT1; ppp) BAX; BAK; BCKDHB; PERK; LPL; LPLA2; GGTA1; CMAH; and PPT1; qqq) BAX; BAK; BCKDHB; MYC; LPL; LPLA2; GGTA1; CMAH; and PPT1; rrr) BAX; BAK; BCKDHB; MYC; PERK; LPL; LPLA2; GGTA1; CMAH; and PPT1; sss) BAX; BAK; BCKDHB; ICAM-1; and SIRT-1; ttt) BAX; BAK; BCKDHB; and ICAM-1; uuu) BAX; BAK; BCKDHA; BCKDHB; ICAM-1; GGTA1; CMAH; LPL; LPLA2; and PPT1; vvv) BAX; BAK; BCKDHA; BCKDHB; ICAM-1; SIRT-1; GGTA1; CMAH; LPL; LPLA2; and PPT1; www) BAX; BAK; BCKDHA; BCKDHB; ICAM-1; SIRT-1; MYC; GGTA1; CMAH; LPL; LPLA2; and PPT1; xxx) BAX; BAK; BCKDHA; BCKDHB; ICAM-1; SIRT-1; MYC; GGTA1; CMAH; LPL, LPLA2; PPT1; and LIPA; yyy) BAX; BAK; BCKDHA; BCKDHB; ICAM-1; GGTA1; CMAH; LPLA2; PPT1; and LIPA; zzz) BAX; BAK; BCKDHA; BCKDHB; ICAM-1; SIRT-1; GGTA1; CMAH; LPLA2; PPT1; and LIPA; aaaa) BAX; BAK; BCKDHA; BCKDHB; ICAM-1; SIRT-1; MYC; GGTA1; CMAH; LPLA2; PPT1; and LIPA; bbbb) BAX; BAK; BCKDHA; BCKDHB; ICAM-1; LPL; LPLA2; and PPT1; cccc) BAX; BAK; BCKDHA; BCKDHB; ICAM-1; SIRT-1; LPL, LPLA2; and PPT1; dddd) BAX; BAK; BCKDHA; BCKDHB; ICAM-1; SIRT-1; MYC; LPL; LPLA2; and PPT1; eeee) BAX; BAK; BCKDHA; BCKDHB; ICAM-1; SIRT-1; MYC; LPL; LPLA2; PPT1; and LIPA; ffff) BAX; BAK; BCKDHA; BCKDHB; ICAM-1; SIRT-1; and MYC; gggg) BAX; BAK; BCKDHA; BCKDHB; ICAM-1; PERK; SIRT-1; and MYC; hhhh) BAX; BAK; BCKDHA; BCKDHB; ICAM-1; PERK; SIRT-1; MYC; GGTA1; CMAH; LPL, LPLA2; PPT1; and LIPA; iiii) BAX; BAK; BCKDHA; BCKDHB; LPL; LPLA2; GGTA1; and CMAH; jjjj) BAX; BAK; BCKDHA; BCKDHB; PERK; LPL; LPLA2; GGTA1; and CMAH; kkkk) BAX; BAK; BCKDHA; BCKDHB; MYC; LPL; LPLA2; GGTA1; and CMAH; llll) BAX; BAK; BCKDHA; BCKDHB; MYC; PERK; LPL; LPLA2; GGTA1; and CMAH; mmmm) BAX; BAK; BCKDHA; BCKDHB; LPL; LPLA2; GGTA1; CMAH; and PPT1; nnnn) BAX; BAK; BCKDHA; BCKDHB; PERK; LPL; LPLA2; GGTA1; CMAH; and PPT1; oooo) BAX; BAK; BCKDHA; BCKDHB; MYC; LPL; LPLA2; GGTA1; CMAH; and PPT1; pppp) BAX; BAK; BCKDHA; BCKDHB; MYC; PERK; LPL; LPLA2; GGTA1; CMAH; and PPT1; qqqq) BAX; BAK; BCKDHA; BCKDHB; ICAM-1; and SIRT-1; or rrrr) BAX; BAK; BCKDHA; BCKDHB; and ICAM-1.

### BRIEF DESCRIPTION OF THE DRAWINGS

FIGS. 1A-1D depict the distribution of expression of three ERV species in RVLP Gag knockout clones and controls (FIG. 1A); the total RVLP titers (sum of three ERVs) in Gag knockout clones and controls (FIG. 1B); the production culture integral viable cell concentration (IVCC) in Gag knockout clones and controls (FIG. 1C); and RVLP titers assessed by TEM in Gag knockout clones and controls (FIG. 1D).

FIG. 2A-2C depict knockout strategies using dual CRISPR/Cas9 targeting nucleases for CHERV-3g and CHERV-1b to produce large deletions (FIG. 2A); and RVLP titers assessed by RT-ddPCR (2B) and TEM (2C) in deletion knockout clones and controls.

### DETAILED DESCRIPTION

The presently disclosed subject matter relates, in certain embodiments, to RVLP-reduced CHO cells suitable to produce recombinant proteins, as well as methods of producing and using such RVLP-reduced CHO production cells. For example, but not by way of limitation, the presently disclosed subject matter is directed, in part, to modified CHO cells and method for producing such modified CHO cells where, prior to the modification, the CHO cells comprise two or more ERV loci selected from:
(a) CHERV-1b (Genbank Accession No. MN527960, SEQ ID NO. 8);
(b) CHERV-2g (Genbank Accession No. MN527961, SEQ ID NO. 9);
(c) ETC109F (30021-39247 of SEQ ID NO. 10);
(d) CHERV-3g (Genbank Accession No. MN527962, SEQ ID NO. 11); and
(e) an ERV locus comprising a sequence having at least 90% identity to any one of (a)-(d), where the modification comprises inactivation, i.e., a reduction or elimination of expression as compared to an unmodified CHO cell, of two or more of the ERV loci of (a)-(e) present in the CHO cell prior to such modification.

For purposes of clarity of disclosure and not by way of limitation, the detailed description is divided into the following subsections:

1. Definitions

2. Inactivation of Endogenous Retrovirus Loci

3. Host Cells

4. Integration of Exogenous Nucleic Acids

5. Preparation and Use of RVLP-Reduced Host Cells

6. Products

7. Examples

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of”, and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “selection marker” can be a gene that allows cells carrying the gene to be specifically selected for or against, in the presence of a corresponding selection agent. For example, but not by way of limitation, a selection marker can allow the host cell transformed with the selection marker gene to be positively selected for in the presence of the gene; a non-transformed host cell would not be capable of growing or surviving under the selective conditions. Selection markers can be positive, negative or bi-functional. Positive selection markers can allow selection for cells carrying the marker, whereas negative selection markers can allow cells carrying the marker to be selectively eliminated. A selection marker can confer resistance to a drug or compensate for a metabolic or catabolic defect in the host cell. In prokaryotic cells, amongst others, genes conferring resistance against ampicillin, tetracycline, kanamycin or chloramphenicol can be used. Resistance genes useful as selection markers in eukaryotic cells include, but are not limited to, genes for aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid. Further marker genes are described in WO 92/08796 and WO 94/28143.

Beyond facilitating a selection in the presence of a corresponding selection agent, a selection marker can alternatively provide a gene encoding a molecule normally not present in the cell, e.g., green fluorescent protein (GFP), enhanced GFP (eGFP), synthetic GFP, yellow fluorescent protein (YFP), enhanced YFP (eYFP), cyan fluorescent protein (CFP), mPlum, mCherry, tdTomato, mStrawberry, J-red, DsRed-monomer, mOrange, mKO, mCitrine, Venus, YPet, Emerald, CyPet, mCFPm, Cerulean, and T-Sapphire. Cells harboring such a gene can be distinguished from cells not harboring this gene, e.g., by the detection of the fluorescence emitted by the encoded polypeptide.

As used herein, the term “operably linked” refers to a juxtaposition of two or more components, wherein the components are in a relationship permitting them to function in their intended manner. For example, a promoter and/or an enhancer is operably linked to a coding sequence if the promoter and/or enhancer acts to modulate the transcription of the coding sequence. In certain embodiments, DNA sequences that are “operably linked” are contiguous and adjacent on a single chromosome. In certain embodiments, e.g., when it is necessary to join two protein encoding regions, such as a secretory leader and a polypeptide, the sequences are contiguous, adjacent, and in the same reading frame. In certain embodiments, an operably linked promoter is located upstream of the coding sequence and can be adjacent to it. In certain embodiments, e.g., with respect to enhancer sequences modulating the expression of a coding sequence, the two components can be operably linked although not adjacent. An enhancer is operably linked to a coding sequence if the enhancer increases transcription of the coding sequence. Operably linked enhancers can be located upstream, within, or downstream of coding sequences and can be located a considerable distance from the promoter of the coding sequence. Operable linkage can be accomplished by recombinant methods known in the art, e.g., using PCR methodology and/or by ligation at convenient restriction sites. If convenient restriction sites do not exist, then synthetic oligonucleotide adaptors or linkers can be used in accord with conventional practice. An internal ribosomal entry site (IRES) is operably linked to an open reading frame (ORF) if it allows initiation of translation of the ORF at an internal location in a 5′ end-independent manner.

As used herein, the term “expression” refers to transcription and/or translation. In certain embodiments, the level of transcription of a desired product can be determined based on the amount of corresponding mRNA that is present. For example, mRNA transcribed from a sequence of interest can be quantitated by PCR or by Northern hybridization. In certain embodiments, protein encoded by a sequence of interest can be quantitated by various methods, e.g. by ELISA, by assaying for the biological activity of the protein, or by employing assays that are independent of such activity, such as Western blotting or radioimmunoassay, using antibodies that recognize and bind to the protein.

The term “antibody” herein is used in the broadest sense and encompasses various antibody structures, including but not limited to monoclonal antibodies, polyclonal antibodies, multispecific antibodies (e.g., bispecific antibodies), half antibodies, and antibody fragments so long as they exhibit a desired antigen-binding activity.

As used herein, the term “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv); and multispecific antibodies formed from antibody fragments. For a review of certain antibody fragments, see Holliger and Hudson, Nature Biotechnology 23:1126-1136 (2005).

As used herein, the term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind to a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, the term “heavy chain” refers to an immunoglobulin heavy chain.

As used herein, the term “light chain” refers to an immunoglobulin light chain.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Multispecific antibodies” are monoclonal antibodies that have binding specificities for at least two different sites, i.e., different epitopes on different antigens or different epitopes on the same antigen. In certain aspects, the multispecific antibody has three or more binding specificities. Multispecific antibodies may be prepared as full-length antibodies or antibody fragments.

The terms “full length antibody”, “intact antibody”, and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

The term “therapeutic antibody” refers to an antibody that is used in the treatment of disease. A therapeutic antibody may have various mechanisms of action. A therapeutic antibody may bind and neutralize the normal function of a target associated with an antigen. For example, a monoclonal antibody that blocks the activity of the of protein needed for the survival of a cancer cell causes the cell's death. Another therapeutic monoclonal antibody may bind and activate the normal function of a target associated with an antigen. For example, a monoclonal antibody can bind to a protein on a cell and trigger an apoptosis signal. Yet another monoclonal antibody may bind to a target antigen expressed only on diseased tissue; conjugation of a toxic payload (effective agent), such as a chemotherapeutic or radioactive agent, to the monoclonal antibody can create an agent for specific delivery of the toxic payload to the diseased tissue, reducing harm to healthy tissue. A “biologically functional fragment” of a therapeutic antibody will exhibit at least one if not some or all of the biological functions attributed to the intact antibody, the function comprising at least specific binding to the target antigen.

The term “diagnostic antibody” refers to an antibody that is used as a diagnostic reagent for a disease. The diagnostic antibody may bind to a target antigen that is specifically associated with, or shows increased expression in, a particular disease. The diagnostic antibody may be used, for example, to detect a target in a biological sample from a patient, or in diagnostic imaging of disease sites, such as tumors, in a patient. A “biologically functional fragment” of a diagnostic antibody will exhibit at least one if not some or all of the biological functions attributed to the intact antibody, the function comprising at least specific binding to the target antigen.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. In certain embodiments, vectors direct the expression of nucleic acids to which they are operatively linked. Such vectors are referred to herein as “expression vectors.”

As used herein, the term “homologous sequences” refers to sequences that share a significant sequence similarity as determined by an alignment of the sequences. For example, two sequences can be about 50%, 60%, 70%, 80%, 90%, 95%, 99%, or 99.9% homologous. The alignment is carried out by algorithms and computer programs including, but not limited to, BLAST, FASTA, and HMME, which compares sequences and calculates the statistical significance of matches based on factors such as sequence length, sequence identify and similarity, and the presence and length of sequence mismatches and gaps. Homologous sequences can refer to both DNA and protein sequences.

As used herein, the term “flanking” refers to that a first nucleotide sequence is located at either a 5′ or 3′ end, or both ends of a second nucleotide sequence. The flanking nucleotide sequence can be adjacent to or at a defined distance from the second nucleotide sequence. There is no specific limit of the length of a flanking nucleotide sequence. For example, a flanking sequence can be a few base pairs or a few thousand base pairs. In certain embodiments, the length of a flanking nucleotide sequence can be about at least 15 base pairs, at least 20 base pairs, at least 30 base pairs, at least 40 base pairs, at least 50 base pairs, at least 75 base pairs, at least 100 base pairs, at least 150 base pairs, at least 200 base pairs, at least 300 base pairs, at least 400 base pairs, at least 500 base pairs, at least 1,000 base pairs, at least 1,500 base pairs, at least 2,000 base pairs, at least 3,000 base pairs, at least 4,000 base pairs, at least 5,000 base pairs, at least 6,000 base pairs, at least 7,000 base pairs, at least 8,000 base pairs, at least 9,000 base pairs, at least 10,000 base pairs.

As used herein, the term “exogenous” indicates that a nucleotide sequence does not originate from a host cell and is introduced into a host cell by traditional DNA delivery methods, e.g., by transfection, electroporation, or transformation methods. The term “endogenous” refers to that a nucleotide sequence originates from a host cell. An “exogenous” nucleotide sequence can have an “endogenous” counterpart that is identical in base compositions, but where the “exogenous” sequence is introduced into the host cell, e.g., via recombinant DNA technology.

### 2. Inactivation of Endogenous Retrovirus Loci

The presently disclosed subject matter relates, in certain embodiments, to RVLP-reduced CHO cells suitable to produce recombinant proteins. Such RVLP-reduced CHO cells can, in certain embodiments, be produced via the inactivation of ERV loci. In certain embodiments, a plurality of ERV loci is inactivated. For example, but not by way of limitation, the presently disclosed subject matter is directed, in part, to modified CHO cells and method for producing such modified CHO cells where, prior to the modification, the CHO cells comprise two or more ERV loci selected from:
(a) CHERV-1b (Genbank Accession No. MN527960, SEQ ID NO. 8);
(b) CHERV-2g (Genbank Accession No. MN527961, SEQ ID NO. 9);
(c) ETC109F (30021-39247 of SEQ ID NO. 10);
(d) CHERV-3g (Genbank Accession No. MN527962, SEQ ID NO. 11); and
(e) an ERV locus comprising a sequence having at least 90% identity to any one of (a)-(d), where the modification comprises inactivation, i.e., a reduction or elimination of expression as compared to an unmodified CHO cell, of two or more of the ERV loci of (a)-(e) present in the CHO cell prior to such modification.

With respect to ETC109F, which corresponds to 30021-39247 of SEQ ID NO. 10 (%′ LTR to 3′ LTR), the remaining portions of SEQ ID NO. 10 correspond to: the CHO-K1 genomic sequence upstream of 5′ end of ETC109F (1-30020 of SEQ ID NO. 10); and: CHO-K1 genomic sequence downstream of 3′ end of ETC109F (39248-59558 of SEQ ID NO. 10). Additional CHERV sequences presented in the context of their genomic loci include CHERV-3g allele A, which is presented as SEQ ID NO. 12, CHERV-3g allele B, which is presented as SEQ ID NO. 13, and CHERV-1b, which is presented as SEQ ID NO. 14.

In certain embodiments, the present disclosure relates methods for inactivation, i.e., a reduction or elimination of expression as compared to an unmodified CHO cell, of two or more ERV loci include, where such inactivation comprises: (1) modification of a gene coding for an ERV protein, e.g., by introducing a deletion, insertion, substitution, or combination thereof into the gene; (2) reducing or eliminating the transcription and/or stability of the mRNA encoding the ERV protein; and (3) reducing or eliminating the translation of the mRNA encoding the ERV protein. In certain embodiments, the reduction or elimination of the ERV protein expression is obtained by targeted genome editing. For example, RNA-guided nuclease-based genome editing systems, e.g., CRISPR/Cas9-based genome editing systems, can be employed to modify one or more target genes, resulting in the reduction or elimination of expression of the ERV gene (or genes) targeted for editing. Additional editing systems, e.g., TALENS, meganucleases, and zinc finger nucleases, can also find use in the methods of editing ERV genes.

In certain embodiments, the inactivation, i.e., a reduction or elimination of expression as compared to an unmodified CHO cell, of two or more ERV loci comprises a knock-out of the respective ERV GAG coding sequence. For example, but not by way of limitation, the knock-out of the respective ERV GAG coding sequence can comprise introduction of an indel into the ERV GAG coding sequence via CRISPR/Cas9-based genome editing. In certain non-limiting embodiments, the knock-out of the respective ERV GAG coding sequence can comprise introduction of a base edit into the ERV GAG coding sequence via CRISPR/Cas9-based base editing. In certain non-limiting embodiments, the knock-out of the respective ERV GAG coding sequence can comprise introduction of deletions flanking the ERV GAG coding sequence via CRISPR/Cas9-based genome editing.

In certain embodiments of the present disclosure, the inactivation, i.e., a reduction or elimination of expression as compared to an unmodified CHO cell, of two or more ERV loci comprises a reduction in the protein expression of an ERV protein product to less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the corresponding ERV protein expression of a reference cell, e.g., a CHO host cell expressing an ERV that has not been inactivated. In certain embodiments, the expression of one or more ERV proteins in a cell that has been modified to reduce or eliminate expression of the ERV protein(s), is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the corresponding endogenous protein expression of a reference cell, e.g., a CHO host cell expressing an ERV that has not been inactivated.

### 3. Host Cells

The presently disclosed subject matter is directed, in certain embodiments, to CHO host cells. In certain embodiments, the CHO host cell is a CHO K1 host cell. In certain embodiments, the CHO host cell is a CHO K1SV host cell. In certain embodiments, the CHO host cell is a DG44 host cell. In certain embodiments, the CHO host cell is a DUKXB-11 host cell. In certain embodiments, the CHO host cell is a CHOK1S host cell. In certain embodiments, the CHO host cell is a CHO KIM host cell.

### 3.1 Host Cells Suitable for Integration of Exogenous Nucleotide Sequences

The presently disclosed subject matter provides, in certain embodiments, modified CHO host cells suitable for targeted integration of exogenous nucleotide sequences. In certain embodiments, the modified CHO host cell comprises an exogenous nucleotide sequence integrated at an integration site on the genome of the host cell, i.e., the host cell is a TI host cell.

An “integration site” comprises a nucleic acid sequence within a host cell genome into which an exogenous nucleotide sequence is inserted. In certain embodiments, an integration site is between two adjacent nucleotides on the host cell genome. In certain embodiments, an integration site includes a stretch of nucleotides between any of which an exogenous nucleotide sequence can be inserted. In certain embodiments, the integration site is located within a specific locus of the genome of the TI host cell. In certain embodiments, the integration site is within an endogenous gene of the TI host cell.

In certain embodiments, the exogenous nucleotide sequence is integrated at a site within a specific locus of the genome of a TI host cell. In certain embodiments, the locus into which the exogenous nucleotide sequence is integrated is at least about 50%, at least about 60%, at least about 70%, at least about 80%, at least about 90%, at least about 95%, at least about 99%, or at least about 99.9% homologous to a sequence selected from SEQ ID Nos. 1-7.

In certain embodiments, the locus into which the exogenous nucleotide sequence is integrated is at least about 50%, at least about 60%, at least about 70%, at least about 80%, at least about 90%, at least about 95%, at least about 99%, or at least about 99.9% homologous to all or a portion of a sequence selected from Contigs NW_006874047.1, NW_006884592.1, NW_006881296.1, NW_003616412.1, NW_003615063.1, NW_006882936.1, and NW 003615411.1.

In certain embodiments, the exogenous nucleotide sequence is integrated at an integration site located within a position selected from nucleotides numbered 1-1,000 bp; 1,000-2,000 bp; 2,000-3,000 bp; 3,000-4,000 bp; and 4,000-4,301 bp of SEQ ID No. 1. In certain embodiments, the exogenous nucleotide sequence is integrated at an integration site located within a position selected from nucleotides numbered 1-100,000 bp; 100,000-200,000 bp; 200,000-300,000 bp; 300,000-400,000 bp; 400,000-500,000 bp; 500,000-600,000 bp; 600,000-700,000 bp; and 700,000-728785 bp of SEQ ID No. 2. In certain embodiments, the exogenous nucleotide sequence is integrated at an integration site located within a position selected from nucleotides numbered 1-100,000 bp; 100,000-200,000 bp; 200,000-300,000 bp; 300,000-400,000 bp; and 400,000-413,983 of SEQ ID No. 3. In certain embodiments, the exogenous nucleotide sequence is integrated at an integration site located within a position selected from nucleotides numbered 1-10,000 bp; 10,000-20,000 bp; 20,000-30,000 bp; and 30,000-30,757 bp of SEQ ID No. 4. In certain embodiments, the exogenous nucleotide sequence is integrated at an integration site located within a position selected from nucleotides numbered 1-10,000 bp; 10,000-20,000 bp; 20,000-30,000 bp; 30,000-40,000 bp; 40,000-50,000 bp; 50,000-60,000 bp; and 60,000-68,962 bp of SEQ ID No. 5. In certain embodiments, the exogenous nucleotide sequence is integrated at an integration site located within a position selected from nucleotides numbered 1-10,000 bp; 10,000-20,000 bp; 20,000-30,000 bp; 30,000-40,000 bp; 40,000-50,000 bp; and 50,000-51,326 bp of SEQ ID No. 6. In certain embodiments, the exogenous nucleotide sequence is integrated at an integration site located within a position selected from nucleotides numbered 1-10,000 bp; 10,000-20,000 bp; and 20,000-22,904 bp of SEQ ID No. 7.

In certain embodiments, the nucleotide immediately 5′ of the integrated exogenous sequence is a nucleotide within the sequence of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, or nucleotides 82214-97705 of NW_003615411.1. In certain embodiments, the nucleotide immediately 5′ of the integrated exogenous sequence is a nucleotide within a sequence of nucleotides sequences at least 50% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, or nucleotides 82214-97705 of NW_003615411.1. In certain embodiments, the nucleotide immediately 5′ of the integrated exogenous sequence is a nucleotide within a sequence of nucleotides at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, or nucleotides 82214-97705 of NW_003615411.1.

In certain embodiments, the nucleotide immediately 3′ of the integrated exogenous sequence is a nucleotide within the sequence of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1. In certain embodiments, the nucleotide immediately 3′ of the integrated exogenous sequence is a nucleotide within a sequence of nucleotides at least 50% homologous to nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1. In certain embodiments, the nucleotide immediately 3′ of the integrated exogenous sequence is a nucleotide within a sequence of nucleotides at least about 50%, at least about 60%, at least about 70%, at least about 80%, at least about 90%, at least about 95%, at least about 99%, or at least about 99.9% homologous to nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, or nucleotides 97706-105117 of NW_003615411.1.

In certain embodiments, the integrated exogenous nucleotide sequence is operably linked to a nucleotide sequence selected from the group consisting of SEQ ID. Nos. 1-7 and sequences at least 50% homologous thereto. In certain embodiments, the nucleotide sequence operably linked to the exogenous nucleotide sequence is at least about 50%, at least about 60%, at least about 70%, at least about 80%, at least about 90%, at least about 95%, at least about 99%, or at least about 99.9% homologous to a sequence selected from SEQ ID Nos. 1-7.

In certain embodiments, the integrated exogenous sequence is flanked 5′ by a nucleotide sequence selected from the group consisting of nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1.and sequences at least 50% homologous thereto, and is flanked 3′ by a nucleotide sequence selected from the group consisting of nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1 and sequences at least 50% homologous thereto. In certain embodiments, the nucleotide sequence flanking 5′ of the integrated exogenous nucleotide sequence is at least about 50%, at least about 60%, at least about 70%, at least about 80%, at least about 90%, at least about 95%, at least about 99%, or at least about 99.9% homologous to nucleotides 41190-45269 of NW_006874047.1, nucleotides 63590-207911 of NW_006884592.1, nucleotides 253831-491909 of NW_006881296.1, nucleotides 69303-79768 of NW_003616412.1, nucleotides 293481-315265 of NW_003615063.1, nucleotides 2650443-2662054 of NW_006882936.1, and nucleotides 82214-97705 of NW_003615411.1, and the nucleotide sequences flanking 3′ of the integrated exogenous nucleotide sequence is at least about 50%, at least about 60%, at least about 70%, at least about 80%, at least about 90%, at least about 95%, at least about 99%, or at least about 99.9% homologous to SEQ ID Nos. nucleotides 45270-45490 of NW_006874047.1, nucleotides 207912-792374 of NW_006884592.1, nucleotides 491910-667813 of NW_006881296.1, nucleotides 79769-100059 of NW_003616412.1, nucleotides 315266-362442 of NW_003615063.1, nucleotides 2662055-2701768 of NW_006882936.1, and nucleotides 97706-105117 of NW_003615411.1.

In certain embodiments, the integrated exogenous nucleotide sequence is integrated within a 20 nucleotide sequence at least about 50%, at least about 60%, at least about 70%, at least about 80%, at least about 90%, at least about 95%, at least about 99%, or at least about 99.9% homologous to a 20 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; and of NW_003615411.1 comprising position 97705.

In certain embodiments, the integrated exogenous nucleotide sequence is integrated within a 50 nucleotide sequence at least about 50%, at least about 60%, at least about 70%, at least about 80%, at least about 90%, at least about 95%, at least about 99%, or at least about 99.9% homologous to a 50 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; and of NW_003615411.1 comprising position 97705.

In certain embodiments, the integrated exogenous nucleotide sequence is integrated within a 100 nucleotide sequence at least about 50%, at least about 60%, at least about 70%, at least about 80%, at least about 90%, at least about 95%, at least about 99%, or at least about 99.9% homologous to a 100 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; and of NW_003615411.1 comprising position 97705.

In certain embodiments, the integrated exogenous nucleotide sequence is integrated within a 200 nucleotide sequence at least about 50%, at least about 60%, at least about 70%, at least about 80%, at least about 90%, at least about 95%, at least about 99%, or at least about 99.9% homologous to a 200 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; and of NW_003615411.1 comprising position 97705.

In certain embodiments, the integrated exogenous nucleotide sequence is integrated within a 500 nucleotide sequence at least about 50%, at least about 60%, at least about 70%, at least about 80%, at least about 90%, at least about 95%, at least about 99%, or at least about 99.9% homologous to a 500 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; and of NW_003615411.1 comprising position 97705.

In certain embodiments, the integrated exogenous nucleotide sequence is integrated within a 1000 nucleotide sequence at least about 50%, at least about 60%, at least about 70%, at least about 80%, at least about 90%, at least about 95%, at least about 99%, or at least about 99.9% homologous to a 1000 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; and of NW_003615411.1 comprising position 97705.

In certain embodiments, the integrated exogenous nucleotide sequence is integrated into a locus immediately adjacent to all or a portion of a sequence selected from the group consisting of sequences at least about 90% homologous to a sequence selected from SEQ ID Nos. 1-7.

In certain embodiments, the integrated exogenous nucleotide sequence is adjacent to a nucleotide sequence selected from the group consisting of SEQ ID. Nos. 1-7 and sequences at least 50% homologous thereto. In certain embodiments, the integrated exogenous nucleotide sequence is within about 100 bp, about 200 bp, about 500 bp, about 1 kb distance from a sequence selected from the group consisting of SEQ ID. Nos. 1-7 and sequences at least 50% homologous thereto.

In certain embodiments, the nucleotide sequence adjacent to the exogenous nucleotide sequence is at least about 50%, at least about 60%, at least about 70%, at least about 80%, at least about 90%, at least about 95%, at least about 99%, or at least about 99.9% homologous to a sequence selected from SEQ ID Nos. 1-7.

In certain embodiments, the exogenous nucleotide sequence is integrated at an integration site adjacent to a position selected from nucleotides numbered 1-1,000 bp; 1,000-2,000 bp; 2,000-3,000 bp; 3,000-4,000 bp; and 4,000-4,301 bp of SEQ ID No. 1. In certain embodiments, the exogenous nucleotide sequence is integrated at an integration site adjacent to a position selected from nucleotides numbered 1-100,000 bp; 100,000-200,000 bp; 200,000-300,000 bp; 300,000-400,000 bp; 400,000-500,000 bp; 500,000-600,000 bp; 600,000-700,000 bp; and 700,000-728785 bp of SEQ ID No. 2. In certain embodiments, the exogenous nucleotide sequence is integrated at an integration site adjacent to a position selected from nucleotides numbered 1-100,000 bp; 100,000-200,000 bp; 200,000-300,000 bp; 300,000-400,000 bp; and 400,000-413,983 of SEQ ID No. 3. In certain embodiments, the exogenous nucleotide sequence is integrated at an integration site adjacent to a position selected from nucleotides numbered 1-10,000 bp; 10,000-20,000 bp; 20,000-30,000 bp; and 30,000-30,757 bp of SEQ ID No. 4. In certain embodiments, the exogenous nucleotide sequence is integrated at an integration site adjacent to a position selected from nucleotides numbered 1-10,000 bp; 10,000-20,000 bp; 20,000-30,000 bp; 30,000-40,000 bp; 40,000-50,000 bp; 50,000-60,000 bp; and 60,000-68,962 bp of SEQ ID No. 5. In certain embodiments, the exogenous nucleotide sequence is integrated at an integration site adjacent to a position selected from nucleotides numbered 1-10,000 bp; 10,000-20,000 bp; 20,000-30,000 bp; 30,000-40,000 bp; 40,000-50,000 bp; and 50,000-51,326 bp of SEQ ID No. 6. In certain embodiments, the exogenous nucleotide sequence is integrated at an integration site adjacent to a position selected from nucleotides numbered 1-10,000 bp; 10,000-20,000 bp; and 20,000-22,904 bp of SEQ ID No. 7.

In certain embodiments, the locus comprising the integration site of the exogenous nucleotide sequence does not encode an open reading frame (ORF). In certain embodiments, the locus comprising the integration site of the exogenous nucleotide sequence includes cis-acting elements, e.g., promoters and enhancers. In certain embodiments, the locus comprising the integration site of the exogenous nucleotide sequence is free of any cis-acting elements, e.g., promoters and enhancers, that enhance gene expression.

In certain embodiments, an exogenous nucleotide sequence is integrated at an integration site within an endogenous gene selected from the group consisting of LOC107977062, LOC100768845, ITPR2, ERE67000.1, UBAP2, MTMR2, and XP_003512331.2. The endogenous LOC107977062, LOC100768845, ITPR2, ERE67000.1, UBAP2, MTMR2, and XP_003512331.2 genes include the wild-type and all homologous sequences of LOC107977062, LOC100768845, ITPR2, ERE67000.1, UBAP2, MTMR2, and XP_003512331.2 genes. In certain embodiments, the homologous sequences of LOC107977062, LOC100768845, ITPR2, ERE67000.1, UBAP2, MTMR2, and XP_003512331.2 genes can be at least about 50%, at least about 60%, at least about 70%, at least about 80%, at least about 90%, at least about 95%, at least about 99%, or at least about 99.9% homologous to the wild-type LOC107977062, LOC100768845, ITPR2, ERE67000.1, UBAP2, MTMR2, and XP_003512331.2 genes. In certain embodiments, the LOC107977062, LOC100768845, ITPR2, ERE67000.1, UBAP2, MTMR2, and XP_003512331.2 genes are wild-type mammalian LOC107977062, LOC100768845, ITPR2, ERE67000.1, UBAP2, MTMR2, and XP_003512331.2 genes. In certain embodiments, the LOC107977062, LOC100768845, ITPR2, ERE67000.1, UBAP2, MTMR2, and XP_003512331.2 genes are wild-type human LOC107977062, LOC100768845, ITPR2, ERE67000.1, UBAP2, MTMR2, and XP_003512331.2 genes. In certain embodiments, the LOC107977062, LOC100768845, ITPR2, ERE67000.1, UBAP2, MTMR2, and XP_003512331.2 genes are wild-type hamster LOC107977062, LOC100768845, ITPR2, ERE67000.1, UBAP2, MTMR2, and XP_003512331.2 genes.

In certain embodiments, the integration site is operably linked to an endogenous gene selected from the group consisting of LOC107977062, LOC100768845, ITPR2, ERE67000.1, UBAP2, MTMR2, XP_003512331.2, and at least about 90% homologous sequences thereof. In certain embodiments, the integration site is flanked by an endogenous gene selected from the group consisting of LOC107977062, LOC100768845, ITPR2, ERE67000.1, UBAP2, MTMR2, XP_003512331.2, and at least about 90% homologous sequences thereof.

Table 1 provides exemplary TI host cell integration sites:

TABLE 1
TI host cell integration sites

| | | Contig | | |
|---|---|---|---|---|
| | | Size | Integration | Gene |
| Host | Contig | (kb) | site (bp) | (SEQ ID No.) |
| 1 | NW_006874047.1 | 727 | 45269 | LOC107977062 |
| | | | | (SEQ ID No. 1) |
| 2 | NW_006884592.1 | 931 | 207911 | LOC100768845 |
| | | | | (SEQ ID No. 2) |
| 3 | NW_006881296.1 | 1016 | 491909 | ITPR2 |
| | | | | (SEQ ID No. 3) |
| 4 | NW_003616412.1 | 127 | 79768 | ERE67000.1 |
| | | | | (SEQ ID No. 4) |
| 5 | NW_003615063.1 | 372 | 315265 | UBAP2 |
| | | | | (SEQ ID No. 5) |
| 6 | NW_006882936.1 | 3042 | 2662054 | MTMR2 |
| | | | | (SEQ ID No. 6) |
| 7 | NW_003615411.1 | 277 | 97706 | XP_003512331.2 |
| | | | | (SEQ ID No. 7) |

In certain embodiments, an integration site and/or the nucleotide sequences flanking the integration site can be identified experimentally. In certain embodiments, an integration site and/or the nucleotide sequences flanking the integration site can be identified by genome-wide screening approaches to isolate host cells that express, at a desirable level, a polypeptide of interest encoded by one or more SOIs integrated into one or more exogenous nucleotide sequences, where the exogenous sequences are themselves integrated into one or more loci in the genome of the host cell. In certain embodiments, an integration site and/or the nucleotide sequences flanking an integration site can be identified by genome-wide screening approaches following transposase-based cassette integration event. In certain embodiments, an integration site and/or the nucleotide sequences flanking an integration site can be identified by brute force random integration screening. In certain embodiments, an integration site and/or the nucleotide sequences flanking an integration site can be determined by conventional sequencing approaches such as target locus amplification (TLA) followed by next-generation sequencing (NGS) and whole-genome NGS. In certain embodiments, the location of an integration site on a chromosome can be determined by conventional cell biology approaches such as fluorescence in-situ hybridization (FISH) analysis.

In certain embodiments, a TI host cell comprises a first exogenous nucleotide sequence integrated at a first integration site within a specific first locus in the genome of the TI host cell and a second exogenous nucleotide sequence integrated at a second integration site within a specific second locus in the genome. In certain embodiments, a TI host cell comprises multiple exogenous nucleotide sequences integrated at multiple integration sites in the genome of the TI host cell.

In certain embodiments, the TI host cells of the present disclosure comprise at least two distinct exogenous nucleotide sequences, e.g., exogenous nucleotide sequences comprising at least one RRS. In certain embodiments, the two or more exogenous nucleotide sequences can be targeted for the introduction of one or more SOIs. In certain embodiments the SOIs are the same. In certain embodiments, the SOIs are distinct. In certain embodiments, a parental TI host cell comprising a first exogenous nucleotide sequence can comprise a second exogenous nucleotide sequence at an integration site that is different from the integration site of the first exogenous nucleotide sequence.

In certain embodiments, the integration sites can be on the same chromosome. In certain embodiments, the integration sites are located within 1-1,000 nucleotides, 1,000-100,000 nucleotides, 100,000-1,000,000 nucleotides or more from each other in the same chromosome. In certain embodiments the integration sites are on different chromosomes. In certain embodiments, a TI host cell comprising an exogenous nucleotide sequence at one integration site can be used for the insertion of at least two, at least three, at least four, at least five, at least six, at least 7, at least 8, or more exogenous nucleotide sequences at the same or different integration sites.

In certain embodiments, the feasibility of recombinase-mediated cassette exchange (RMCE) of at least two integration sites can be evaluated for each site individually. In certain embodiments, the feasibility of RMCE at least two integration sites can be evaluated simultaneously. The feasibility of RMCE at multiple sites can evaluated by methods known in the art, e.g., measuring the polypeptide titer, or the polypeptide specific production. In certain embodiments, the evaluation can be performed by methods known in the art, e.g., by evaluating the titer and/or specific productivity of a culture of the TI host cell expressing the SOI(s). Exemplary culture strategies include, but are not limited to, fed-batch shake flask cultures and a bioreactor fed-batch cultures. Titer and specific productivity of the TI host cells expressing a polypeptide of interest can evaluated by methods known in the art, e.g., but not limited to, ELISA, FACS, Fluorometric Microvolume Assay Technology (FMAT), protein-A affinity chromatography, Western blot analysis.

### 3.2 Reduced or Eliminated Expression of Endogenous Host Cell Proteins

In certain embodiments, the present disclosure relates to modified CHO cells, e.g., CHO cells, where the expression of one or more CHO cell endogenous proteins is reduced or eliminated. For example, but not by way of limitation, methods for reducing or eliminating endogenous protein expression in a CHO cell include: (1) modification of a gene coding for the endogenous protein or component thereof, e.g., by introducing a deletion, insertion, substitution, or combination thereof into the gene; (2) reducing or eliminating the transcription and/or stability of the mRNA encoding the endogenous protein or a component thereof, and (3) reducing or eliminating the translation of the mRNA encoding the endogenous protein or a component thereof. In certain embodiments, the reduction or elimination of protein expression is obtained by targeted genome editing. For example, CRISPR/Cas9-based genome editing can be employed to modify one or more target genes, resulting in the reduction or elimination of expression of the gene (or genes) targeted for editing.

In certain embodiments, one or more of the CHO cell endogenous proteins targeted for reduced or eliminated expression are selected based on their role in promoting apoptosis. As apoptosis can decrease culture viability and productivity, reducing or eliminating expression of such proteins can positively impact culture viability and productivity. For example, but not by way of limitation, the CHO cell protein selected based on its role in promoting apoptosis is BCL2 Associated X, Apoptosis Regulator (BAX) or BCL2 Antagonist/Killer 1 (BAK). In certain embodiments, the CHO cells of the present disclosure exhibit reduced or eliminated expression of BAX. In certain embodiments, the CHO cells of the present disclosure exhibit reduced or eliminated expression of BAK. In certain embodiments, the CHO cells of the present disclosure exhibit reduced or eliminated expression of BAX and BAK.

In certain embodiments, the CHO cell endogenous product targeted for reduced or eliminated expression is selected based on its role in promoting clumping and/or aggregation during cell culture. When CHO cells are used for production of a recombinant protein of interest, such clumping and/or aggregation during cell culture can lead to reduced protein titers due to the negative impact of clumping and/or aggregation on CHO cell viability. For example, but not by way of limitation, the CHO cell endogenous protein selected based on its role in promoting clumping and/or aggregation during cell culture is Intercellular Adhesion Molecule 1 (ICAM-1). In certain embodiments, the CHO cells of the present disclosure exhibit reduced or eliminated expression of ICAM-1.

In certain embodiments, the CHO cell endogenous protein that is targeted for reduced or eliminated expression is an endogenous protein that is selected based on its role in regulating the unfolded protein response (UPR). For example, but not by way of limitation, the cellular protein selected based on its role in regulating the UPR is inositol-requiring enzyme 1 (IRE1), protein kinase R-like ER kinase (PERK) or activating transcription factor 6 (ATF6). In certain embodiments, the modified cells of the present disclosure exhibit reduced or eliminated expression of PERK. In certain embodiments, PERK, as used herein, refers to a eukaryotic PERK cellular protein, e.g., the CHO PERK cellular protein (Gene ID: 100765343; GenBank: EGW03658.1; and isoforms NCBI Reference Sequence: XP_027285344.2 and NCBI Reference Sequence: XP_016831844.1), and functional variants thereof. In certain embodiments, functional variants of PERK, as used herein encompass PERK sequence variants having 50%, 55%, 60%, 65%, 70%, 75%, 80%, 85%, 90%, 91%, 92%, 93%, 94%, 95%, 96%, 97%, 98%, 99% identity to the wild type PERK sequence of the modified cell used for the production of a recombinant protein of interest.

In certain embodiments, one or more of the CHO cell endogenous proteins targeted for reduced or eliminated expression are selected based on their role in promoting inefficient cell growth. CHO cells express many endogenous proteins that are not essential for cell growth, survival, and/or productivity. Because expression of these endogenous proteins consumes considerable cellular energy and DNA/protein building blocks, reducing or eliminating the expression of such endogenous proteins can render cell growth more efficient and, in the case of cells used to produce a recombinant protein of interest, those cellular resources can be diverted to achieve higher productivity of the recombinant protein of interest. For example, but not by way of limitation, the CHO cell endogenous protein selected based on its role in promoting efficient cell growth and higher productivity of a recombinant protein of interest is BAX, BAK, ICAM-1, PERK, Sirtuin 1 (SIRT-1) or MYC Proto-Oncogene, BHLH Transcription Factor (MYC). In certain embodiments, the CHO cells of the present disclosure exhibit reduced or eliminated expression of SIRT-1. In certain embodiments, the CHO cells of the present disclosure exhibit reduced or eliminated expression of PERK. In certain embodiments, the CHO cells of the present disclosure exhibit reduced or eliminated expression of MYC. In certain embodiments, the CHO cells of the present disclosure exhibit reduced or eliminated expression of SIRT-1 and MYC. In certain embodiments, the CHO cells of the present disclosure exhibit reduced or eliminated expression of BAX and MYC. In certain embodiments, the CHO cells of the present disclosure exhibit reduced or eliminated expression of BAK and MYC. In certain embodiments, the CHO cells of the present disclosure exhibit reduced or eliminated expression of ICAM-1 and MYC. In certain embodiments, the CHO cells of the present disclosure exhibit reduced or eliminated expression of BAX and SIRT-1. In certain embodiments, the CHO cells of the present disclosure exhibit reduced or eliminated expression of BAK and SIRT-1. In certain embodiments, the CHO cells of the present disclosure exhibit reduced or eliminated expression of ICAM-1 and SIRT-1. In certain embodiments, the CHO cells of the present disclosure exhibit reduced or eliminated expression of BAX, SIRT-1, and MYC. In certain embodiments, the CHO cells of the present disclosure exhibit reduced or eliminated expression of BAK, SIRT-1, and MYC. In certain embodiments, the CHO cells of the present disclosure exhibit reduced or eliminated expression of ICAM-1, SIRT-1, and MYC. In certain embodiments, the CHO cells of the present disclosure exhibit reduced or eliminated expression of BAX, BAK, SIRT-1, and MYC. In certain embodiments, the CHO cells of the present disclosure exhibit reduced or eliminated expression of BAX, ICAM-1, SIRT-1, and MYC. In certain embodiments, the CHO cells of the present disclosure exhibit reduced or eliminated expression of BAK, ICAM-1, SIRT-1, and MYC. In certain embodiments, the CHO cells of the present disclosure exhibit reduced or eliminated expression of BAX, BAK, MYC, SIRT-1, and ICAM. In certain embodiments, the CHO cells of the present disclosure exhibit reduced or eliminated expression of BAX, BAK, ICAM-1, PERK, SIRT-1, and/or MYC.

In certain embodiments, the CHO cell endogenous protein that is targeted for reduced or eliminated expression is an endogenous protein that can promote non-human glycosylation patterns in a recombinant protein product, e.g., when the cell is used for recombinant protein production. Such non-human glycosylation patterns can include the addition of Galactose-α-1,3-galactose (αGAL) and/or N-glycolylneuraminic acid (NGNA). For example, but not by way of limitation, the CHO cell protein selected based on its role in promoting non-human glycosylation patterns is Glycoprotein Alpha-Galactosyltransferase 1 (GGTA1), which promotes αGAL addition, or Cytidine Monophosphate-N-Acetylneuraminic Acid Hydroxylase (CMAH), which promotes NGNA addition. In certain embodiments, the CHO cells of the present disclosure exhibit reduced or eliminated expression of GGTA1. In certain embodiments, the CHO cells of the present disclosure exhibit reduced or eliminated expression of CMAH. In certain embodiments, the CHO cells of the present disclosure exhibit reduced or eliminated expression of GGTA1 and CMAH.

In certain embodiments, the CHO cell endogenous protein that is targeted for reduced or eliminated expression is an endogenous protein that promotes the catabolism of branched chain amino acids (BCAAs). While branched chain amino acids, e.g., leucine, isoleucine, and valine, are essential amino acids and are thus generally included in chemically defined media employed in CHO cell culture, the catabolism of BCAAs can lead to toxic intermediates and metabolites that decrease cell growth, productivity and protein quality. For example, the CHO cell protein selected based on its role in promoting BCAA catabolism is Branched chain keto acid dehydrogenase E1 alpha subunit (BCKDHA) or Branched-chain alpha-keto acid dehydrogenase E1 beta subunit (BCKDHB).

In contexts where the cell is used for production of a recombinant protein of interest, certain CHO cell endogenous proteins can co-purify with the protein of interest, leading to increased costs associated with additional purification processes and/or decreased shelf-life of the resulting recombinant product. For example, certain residual host cell proteins that co-purify with the recombinant protein of interest can degrade polysorbate used as a surfactant in the final drug product, and lead to particle formation. Thus, in certain embodiments, the CHO cell endogenous host cell proteins targeted for reduced or eliminated expression based on their potential to co-purify with the recombinant protein of interest and degrade polysorbate used as a surfactant in the final drug product include Lipoprotein lipase (LPL) which is also referred to as LPL1; Phospholipase A2 group (LPLA2) which is also referred to as PLA2G7; Palmitoyl-protein thioesterase 1 (PPT1); or Lipase A (Lysosomal acid lipase/cholesteryl ester hydrolase, Lipase) (LIPA). In certain embodiments, the CHO cells of the present disclosure exhibit reduced or eliminated expression of PPT1. In certain embodiments, the CHO cells of the present disclosure exhibit reduced or eliminated expression of PPT1 and LPL. In certain embodiments, the CHO cells of the present disclosure exhibit reduced or eliminated expression of LPLA2. In certain embodiments, the CHO cells of the present disclosure exhibit reduced or eliminated expression of PPT1 and LIPA. In certain embodiments, the CHO cells of the present disclosure exhibit reduced or eliminated expression of PPT1, LPL and LPLA2. In certain embodiments, the CHO cells of the present disclosure exhibit reduced or eliminated expression of PPT1, LPL and LIPA. In certain embodiments, the CHO cells of the present disclosure exhibit reduced or eliminated expression of PPT1, LIPA and LPLA2. In certain embodiments, the CHO cells of the present disclosure exhibit reduced or eliminated expression of PPT1, LPL, LIPA, and LPLA2.

In certain embodiments, the host cells of the present disclosure exhibit reduced or eliminated expression of one or more endogenous proteins in order to facilitate purification of a recombinant protein of interest by reducing the overall amount of host cell endogenous protein produced during cell culture. Such reduction in overall host cell endogenous protein production can reduce the burden on the chromatographic and other materials and systems employed in the purification process, thereby reducing the overall cost of purification and increasing purification process efficiency. For example, but not by way of limitation, the host cell endogenous protein targeted for reduced or eliminated expression based on the overall amount of the endogenous protein produced during cell culture is selected from the following endogenous product: MYC Proto-Oncogene, BHLH Transcription Factor (MYC); BCL2 Associated X, Apoptosis Regulator (BAX); BCL2 Antagonist/Killer 1 (BAK); Intercellular Adhesion Molecule 1 (ICAM-1); Protein Kinase R-like ER Kinase (PERK); Sirtuin 1 (SIRT-1); Glycoprotein Alpha-Galactosyltransferase 1 (GGTA1); Cytidine Monophosphate-N-Acetylneuraminic Acid Hydroxylase (CMAH); Lipoprotein lipase (LPL); Phospholipase A2 group (LPLA2); Palmitoyl-protein thioesterase 1 (PPT1); Branched Chain Keto Acid Dehydrogenase E1 alpha subunit (BCKDHA); Branched Chain Keto Acid Dehydrogenase E1 beta subunit (BCKDHB); and Lipase A (Lysosomal acid lipase/cholesteryl ester hydrolase, Lipase) (LIPA).

In certain embodiments, the host cells of the present disclosure exhibit reduced or eliminated expression of the following endogenous proteins: BAX; BAK; ICAM-1; GGTA1; CMAH; LPL; LPLA2; and PPT1.

In certain embodiments, the host cells of the present disclosure exhibit reduced or eliminated expression of the following endogenous proteins: BAX; BAK; ICAM-1; SIRT-1; GGTA1; CMAH; LPL; LPLA2; and PPT1.

In certain embodiments, the host cells of the present disclosure exhibit reduced or eliminated expression of the following endogenous proteins: BAX; BAK; ICAM-1; SIRT-1; MYC; GGTA1; CMAH; LPL; LPLA2; and PPT1.

In certain embodiments, the host cells of the present disclosure exhibit reduced or eliminated expression of the following endogenous proteins: BAX; BAK; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous proteins: BAX; BAK; ICAM-1; GGTA1; CMAH; LPLA2; PPT1; and LIPA.

In certain embodiments, the host cells of the present disclosure exhibit reduced or eliminated expression of the following endogenous proteins: BAX; BAK; ICAM-1; SIRT-1; GGTA1; CMAH; LPLA2; PPT1; and LIPA.

In certain embodiments, the host cells of the present disclosure exhibit reduced or eliminated expression of the following endogenous proteins: BAX; BAK; ICAM-1; SIRT-1; MYC; GGTA1; CMAH; LPLA2; PPT1; and LIPA.

In certain embodiments, the host cells of the present disclosure exhibit reduced or eliminated expression of the following endogenous proteins: BAX; BAK; ICAM-1; LPL; LPLA2; and PPT1.

In certain embodiments, the host cells of the present disclosure exhibit reduced or eliminated expression of the following endogenous proteins: BAX; BAK; ICAM-1; SIRT-1; LPL, LPLA2; and PPT1.

In certain embodiments, the host cells of the present disclosure exhibit reduced or eliminated expression of the following endogenous proteins: BAX; BAK; ICAM-1; SIRT-1; MYC; LPL; LPLA2; and PPT1.

In certain embodiments, the host cells of the present disclosure exhibit reduced or eliminated expression of the following endogenous proteins: BAX; BAK; ICAM-1; SIRT-1; MYC; LPL; LPLA2; PPT1; and LIPA.

In certain embodiments, the host cells of the present disclosure exhibit reduced or eliminated expression of the following endogenous proteins: BAX; BAK; ICAM-1; PERK; SIRT-1; and MYC.

In certain embodiments, the host cells of the present disclosure exhibit reduced or eliminated expression of the following endogenous proteins: MYC; BAX; BAK; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous proteins: BAX; BAK; and PERK.

In certain embodiments, the host cells of the present disclosure exhibit reduced or eliminated expression of the following endogenous proteins: BAX; BAK; MYC; SIRT-1; and ICAM.

In certain embodiments, the host cells of the present disclosure exhibit reduced or eliminated expression of the following endogenous proteins: BAX; BAK; LPL; LPLA2; GGTA1; and CMAH.

In certain embodiments, the host cells of the present disclosure exhibit reduced or eliminated expression of the following endogenous proteins: BAX; BAK; PERK; LPL; LPLA2; GGTA1; and CMAH.

In certain embodiments, the host cells of the present disclosure exhibit reduced or eliminated expression of the following endogenous proteins: BAX; BAK; MYC; LPL; LPLA2; GGTA1; and CMAH.

In certain embodiments, the host cells of the present disclosure exhibit reduced or eliminated expression of the following endogenous proteins: BAX; BAK; MYC; PERK; LPL; LPLA2; GGTA1; and CMAH.

In certain embodiments, the host cells of the present disclosure exhibit reduced or eliminated expression of the following endogenous proteins: BAX; BAK; LPL; LPLA2; GGTA1; CMAH; and PPT1.

In certain embodiments, the host cells of the present disclosure exhibit reduced or eliminated expression of the following endogenous proteins: BAX; BAK; PERK; LPL; LPLA2; GGTA1; CMAH; and PPT1.

In certain embodiments, the host cells of the present disclosure exhibit reduced or eliminated expression of the following endogenous proteins: BAX; BAK; MYC; LPL; LPLA2; GGTA1; CMAH; and PPT1.

In certain embodiments, the host cells of the present disclosure exhibit reduced or eliminated expression of the following endogenous proteins: BAX; BAK; MYC; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHA; ICAM-1; GGTA1; CMAH; LPL; LPLA2; and PPT1.

In certain embodiments, the host cells of the present disclosure exhibit reduced or eliminated expression of the following endogenous proteins: BAX; BAK; BCKDHA; ICAM-1; SIRT-1; GGTA1; CMAH; LPL; LPLA2; and PPT1.

In certain embodiments, the host cells of the present disclosure exhibit reduced or eliminated expression of the following endogenous proteins: BAX; BAK; BCKDHA; ICAM-1; SIRT-1; MYC; GGTA1; CMAH; LPL; LPLA2; and PPT1.

In certain embodiments, the host cells of the present disclosure exhibit reduced or eliminated expression of the following endogenous proteins: BAX; BAK; BCKDHA; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous proteins: BAX; BAK; BCKDHA; ICAM-1; GGTA1; CMAH; LPLA2; PPT1; and LIPA.

In certain embodiments, the host cells of the present disclosure exhibit reduced or eliminated expression of the following endogenous proteins: BAX; BAK; BCKDHA; ICAM-1; SIRT-1; GGTA1; CMAH; LPLA2; PPT1; and LIPA.

In certain embodiments, the host cells of the present disclosure exhibit reduced or eliminated expression of the following endogenous proteins: BAX; BAK; BCKDHA; ICAM-1; SIRT-1; MYC; GGTA1; CMAH; LPLA2; PPT1; and LIPA.

In certain embodiments, the host cells of the present disclosure exhibit reduced or eliminated expression of the following endogenous proteins: BAX; BAK; BCKDHA; ICAM-1; LPL; LPLA2; and PPT1.

In certain embodiments, the host cells of the present disclosure exhibit reduced or eliminated expression of the following endogenous proteins: BAX; BAK; BCKDHA; ICAM-1; SIRT-1; LPL, LPLA2; and PPT1.

In certain embodiments, the host cells of the present disclosure exhibit reduced or eliminated expression of the following endogenous proteins: BAX; BAK; BCKDHA; ICAM-1; SIRT-1; MYC; LPL; LPLA2; and PPT1.

In certain embodiments, the host cells of the present disclosure exhibit reduced or eliminated expression of the following endogenous proteins: BAX; BAK; BCKDHA; ICAM-1; SIRT-1; MYC; LPL; LPLA2; PPT1; and LIPA.

In certain embodiments, the host cells of the present disclosure exhibit reduced or eliminated expression of the following endogenous proteins: BAX; BAK; BCKDHA; ICAM-1; PERK; SIRT-1; and MYC.

In certain embodiments, the host cells of the present disclosure exhibit reduced or eliminated expression of the following endogenous proteins: MYC; BAX; BAK; BCKDHA; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous proteins: BAX; BAK; BCKDHA; and PERK.

In certain embodiments, the host cells of the present disclosure exhibit reduced or eliminated expression of the following endogenous proteins: BAX; BAK; BCKDHA; MYC; SIRT-1; and ICAM.

In certain embodiments, the host cells of the present disclosure exhibit reduced or eliminated expression of the following endogenous proteins: BAX; BAK; BCKDHA; LPL; LPLA2; GGTA1; and CMAH.

In certain embodiments, the host cells of the present disclosure exhibit reduced or eliminated expression of the following endogenous proteins: BAX; BAK; BCKDHA; PERK; LPL; LPLA2; GGTA1; and CMAH.

In certain embodiments, the host cells of the present disclosure exhibit reduced or eliminated expression of the following endogenous proteins: BAX; BAK; BCKDHA; MYC; LPL; LPLA2; GGTA1; and CMAH.

In certain embodiments, the host cells of the present disclosure exhibit reduced or eliminated expression of the following endogenous proteins: BAX; BAK; BCKDHA; MYC; PERK; LPL; LPLA2; GGTA1; and CMAH.

In certain embodiments, the host cells of the present disclosure exhibit reduced or eliminated expression of the following endogenous proteins: BAX; BAK; BCKDHA; LPL; LPLA2; GGTA1; CMAH; and PPT1.

In certain embodiments, the host cells of the present disclosure exhibit reduced or eliminated expression of the following endogenous proteins: BAX; BAK; BCKDHA; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHA; MYC; LPL; LPLA2; GGTA1; CMAH; and PPT1.

In certain embodiments, the host cells of the present disclosure exhibit reduced or eliminated expression of the following endogenous proteins: BAX; BAK; BCKDHA; MYC; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHB; MYC; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHB; ICAM-1; GGTA1; CMAH; LPL; LPLA2; and PPT1.

In certain embodiments, the host cells of the present disclosure exhibit reduced or eliminated expression of the following endogenous proteins: BAX; BAK; BCKDHB; ICAM-1; SIRT-1; GGTA1; CMAH; LPL; LPLA2; and PPT1.

In certain embodiments, the host cells of the present disclosure exhibit reduced or eliminated expression of the following endogenous proteins: BAX; BAK; BCKDHB; ICAM-1; SIRT-1; MYC; GGTA1; CMAH; LPL; LPLA2; and PPT1.

In certain embodiments, the host cells of the present disclosure exhibit reduced or eliminated expression of the following endogenous proteins: BAX; BAK; BCKDHB; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous proteins: BAX; BAK; BCKDHB; ICAM-1; GGTA1; CMAH; LPLA2; PPT1; and LIPA.

In certain embodiments, the host cells of the present disclosure exhibit reduced or eliminated expression of the following endogenous proteins: BAX; BAK; BCKDHB; ICAM-1; SIRT-1; GGTA1; CMAH; LPLA2; PPT1; and LIPA.

In certain embodiments, the host cells of the present disclosure exhibit reduced or eliminated expression of the following endogenous proteins: BAX; BAK; BCKDHB; ICAM-1; SIRT-1; MYC; GGTA1; CMAH; LPLA2; PPT1; and LIPA.

In certain embodiments, the host cells of the present disclosure exhibit reduced or eliminated expression of the following endogenous proteins: BAX; BAK; BCKDHB; ICAM-1; LPL; LPLA2; and PPT1.

In certain embodiments, the host cells of the present disclosure exhibit reduced or eliminated expression of the following endogenous proteins: BAX; BAK; BCKDHB; ICAM-1; SIRT-1; LPL, LPLA2; and PPT1.

In certain embodiments, the host cells of the present disclosure exhibit reduced or eliminated expression of the following endogenous proteins: BAX; BAK; BCKDHB; ICAM-1; SIRT-1; MYC; LPL; LPLA2; and PPT1.

In certain embodiments, the host cells of the present disclosure exhibit reduced or eliminated expression of the following endogenous proteins: BAX; BAK; BCKDHB; ICAM-1; SIRT-1; MYC; LPL; LPLA2; PPT1; and LIPA.

In certain embodiments, the host cells of the present disclosure exhibit reduced or eliminated expression of the following endogenous proteins: BAX; BAK; BCKDHB; ICAM-1; PERK; SIRT-1; and MYC.

In certain embodiments, the host cells of the present disclosure exhibit reduced or eliminated expression of the following endogenous proteins: MYC; BAX; BAK; BCKDHB; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous proteins: BAX; BAK; BCKDHB; and PERK.

In certain embodiments, the host cells of the present disclosure exhibit reduced or eliminated expression of the following endogenous proteins: BAX; BAK; BCKDHB; MYC; SIRT-1; and ICAM.

In certain embodiments, the host cells of the present disclosure exhibit reduced or eliminated expression of the following endogenous proteins: BAX; BAK; BCKDHB; LPL; LPLA2; GGTA1; and CMAH.

In certain embodiments, the host cells of the present disclosure exhibit reduced or eliminated expression of the following endogenous proteins: BAX; BAK; BCKDHB; PERK; LPL; LPLA2; GGTA1; and CMAH.

In certain embodiments, the host cells of the present disclosure exhibit reduced or eliminated expression of the following endogenous proteins: BAX; BAK; BCKDHB; MYC; LPL; LPLA2; GGTA1; and CMAH.

In certain embodiments, the host cells of the present disclosure exhibit reduced or eliminated expression of the following endogenous proteins: BAX; BAK; BCKDHB; MYC; PERK; LPL; LPLA2; GGTA1; and CMAH.

In certain embodiments, the host cells of the present disclosure exhibit reduced or eliminated expression of the following endogenous proteins: BAX; BAK; BCKDHB; LPL; LPLA2; GGTA1; CMAH; and PPT1.

In certain embodiments, the host cells of the present disclosure exhibit reduced or eliminated expression of the following endogenous proteins: BAX; BAK; BCKDHB; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHB; MYC; LPL; LPLA2; GGTA1; CMAH; and PPT1.

In certain embodiments, the host cells of the present disclosure exhibit reduced or eliminated expression of the following endogenous proteins: BAX; BAK; BCKDHB; MYC; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHA; BCKDHB; MYC; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHA; BCKDHB; ICAM-1; GGTA1; CMAH; LPL; LPLA2; and PPT1.

In certain embodiments, the host cells of the present disclosure exhibit reduced or eliminated expression of the following endogenous proteins: BAX; BAK; BCKDHA; BCKDHB; ICAM-1; SIRT-1; GGTA1; CMAH; LPL; LPLA2; and PPT1.

In certain embodiments, the host cells of the present disclosure exhibit reduced or eliminated expression of the following endogenous proteins: BAX; BAK; BCKDHA; BCKDHB; ICAM-1; SIRT-1; MYC; GGTA1; CMAH; LPL; LPLA2; and PPT1.

In certain embodiments, the host cells of the present disclosure exhibit reduced or eliminated expression of the following endogenous proteins: BAX; BAK; BCKDHA; BCKDHB; ICAM-1; SIRT-1; MYC; GGTA1; CMAH; LPL, LPLA2; PPT1; and LIPA.

In certain embodiments, the host cells of the present disclosure exhibit reduced or eliminated expression of the following endogenous proteins: BAX; BAK; BCKDHA; BCKDHB; ICAM-1; GGTA1; CMAH; LPLA2; PPT1; and LIPA.

In certain embodiments, the host cells of the present disclosure exhibit reduced or eliminated expression of the following endogenous proteins: BAX; BAK; BCKDHA; BCKDHB; ICAM-1; SIRT-1; GGTA1; CMAH; LPLA2; PPT1; and LIPA.

In certain embodiments, the host cells of the present disclosure exhibit reduced or eliminated expression of the following endogenous proteins: BAX; BAK; BCKDHA; BCKDHB; ICAM-1; SIRT-1; MYC; GGTA1; CMAH; LPLA2; PPT1; and LIPA.

In certain embodiments, the host cells of the present disclosure exhibit reduced or eliminated expression of the following endogenous proteins: BAX; BAK; BCKDHA; BCKDHB; ICAM-1; LPL; LPLA2; and PPT1.

In certain embodiments, the host cells of the present disclosure exhibit reduced or eliminated expression of the following endogenous proteins: BAX; BAK; BCKDHA; BCKDHB; ICAM-1; SIRT-1; LPL, LPLA2; and PPT1.

In certain embodiments, the host cells of the present disclosure exhibit reduced or eliminated expression of the following endogenous proteins: BAX; BAK; BCKDHA; BCKDHB; ICAM-1; SIRT-1; MYC; LPL; LPLA2; and PPT1.

In certain embodiments, the host cells of the present disclosure exhibit reduced or eliminated expression of the following endogenous proteins: BAX; BAK; BCKDHA; BCKDHB; ICAM-1; SIRT-1; MYC; LPL; LPLA2; PPT1; and LIPA.

In certain embodiments, the host cells of the present disclosure exhibit reduced or eliminated expression of the following endogenous proteins: BAX; BAK; BCKDHA; BCKDHB; ICAM-1; PERK; SIRT-1; and MYC.

In certain embodiments, the host cells of the present disclosure exhibit reduced or eliminated expression of the following endogenous proteins: MYC; BAX; BAK; BCKDHA; BCKDHB; ICAM-1; PERK; SIRT-1; GGTA1; CMAH; LPL; LPLA2; PPT1 and LIPA.

In certain embodiments, the host cells of the present disclosure exhibit reduced or eliminated expression of the following endogenous proteins: BAX; BAK; BCKDHA; BCKDHB; and PERK.

In certain embodiments, the host cells of the present disclosure exhibit reduced or eliminated expression of the following endogenous proteins: BAX; BAK; BCKDHA; BCKDHB; MYC; SIRT-1; and ICAM.

In certain embodiments, the host cells of the present disclosure exhibit reduced or eliminated expression of the following endogenous proteins: BAX; BAK; BCKDHA; BCKDHB; LPL; LPLA2; GGTA1; and CMAH.

In certain embodiments, the host cells of the present disclosure exhibit reduced or eliminated expression of the following endogenous proteins: BAX; BAK; BCKDHA; BCKDHB; PERK; LPL; LPLA2; GGTA1; and CMAH.

In certain embodiments, the host cells of the present disclosure exhibit reduced or eliminated expression of the following endogenous proteins: BAX; BAK; BCKDHA; BCKDHB; MYC; LPL; LPLA2; GGTA1; and CMAH.

In certain embodiments, the host cells of the present disclosure exhibit reduced or eliminated expression of the following endogenous proteins: BAX; BAK; BCKDHA; BCKDHB; MYC; PERK; LPL; LPLA2; GGTA1; and CMAH.

In certain embodiments, the host cells of the present disclosure exhibit reduced or eliminated expression of the following endogenous proteins: BAX; BAK; BCKDHA; BCKDHB; LPL; LPLA2; GGTA1; CMAH; and PPT1.

In certain embodiments, the host cells of the present disclosure exhibit reduced or eliminated expression of the following endogenous proteins: BAX; BAK; BCKDHA; BCKDHB; PERK; LPL; LPLA2; GGTA1; CMAH; and PPT1.

In certain embodiments, the host cells of the present disclosure exhibit reduced or eliminated expression of the following endogenous proteins: BAX; BAK; BCKDHA; BCKDHB; MYC; LPL; LPLA2; GGTA1; CMAH; and PPT1.

In certain embodiments, the host cells of the present disclosure exhibit reduced or eliminated expression of the following endogenous proteins. BAX; BAK; BCKDHA; BCKDHA; BCKDHB; MYC; PERK; LPL; LPLA2; GGTA1; CMAH; and PPT1.

In certain embodiments, a host cell of the present disclosure is modified to reduce or eliminate the expression of one or more host cell endogenous proteins relative to the expression of the host cell endogenous proteins in an unmodified, i.e., “reference”, host cell. In certain embodiments, the reference host cells are host cells where the expression of one or more particular endogenous product, e.g., a BAX; BAK; ICAM-1; SIRT-1; MYC; GGTA1; CMAH; LPL; LIPA; LPLA2; BCKDHA; BCKDHB; PPT1; and/or PERK polypeptide, is not reduced or eliminated. In certain embodiments, a reference host cell is a cell that comprises at least one or both wild-type alleles of the gene(s) coding for BAX; BAK; ICAM-1; SIRT-1; MYC; GGTA1; CMAH; LPL; LIPA; LPLA2; BCKDHA; BCKDHB; PPT1; and/or PERK. For example, but not by way of limitation, a reference host cell is a host cell that has both wild-type alleles of the gene(s) coding for BAX; BAK; ICAM-1; SIRT-1; MYC; GGTA1; CMAH; LPL; LIPA; LPLA2; BCKDHA; BCKDHB; PPT1; and/or PERK. In certain embodiments, the reference host cells are WT host cells. In certain embodiments, the modification of reducing or eliminating the expression of one or more host cell endogenous proteins is performed before the introduction of the exogenous nucleic acid encoding the recombinant protein of interest. In certain embodiments, the modification of reducing or eliminating the expression of one or more host cell endogenous proteins is performed after the introduction of the exogenous nucleic acid encoding the recombinant protein of interest.

In certain embodiments, the expression of one or more endogenous proteins, e.g., a BAX; BAK; ICAM-1; SIRT-1; MYC; GGTA1; CMAH; LPL; LPLA2; BCKDHA; BCKDHB; PPT1;
and/or PERK polypeptide, in a cell that has been modified to reduce or eliminate expression of the endogenous product, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the corresponding endogenous protein expression of a reference cell, e.g., a WT host cell. In certain embodiments, the expression of one or more endogenous proteins in a cell that has been modified to reduce or eliminate expression of the endogenous proteins, is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2%, or less than about 1% of the corresponding endogenous protein expression of a reference cell, e.g., a WT host cell.

In certain embodiments, the expression of one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in a host cell that has been modified to reduce or eliminate expression of the endogenous proteins, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2% or at least about 1% of the corresponding endogenous protein expression of a reference host cell, e.g., a WT host cell. In certain embodiments, the expression of one or more endogenous proteins in a host cell that has been modified to reduce or eliminate expression of the endogenous product, is at least about 90%, at least about 80%, at least about 70%, at least about 60%, at least about 50%, at least about 40%, at least about 30%, at least about 20%, at least about 10%, at least about 5%, at least about 4%, at least about 3%, at least about 2%, or at least about 1% of the corresponding endogenous protein expression of a reference cell, e.g., a WT CHO cell.

In certain embodiments, the expression of one or more particular endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous proteins, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous protein expression of a reference host cell, e.g., a WT host cell. In certain embodiments, the expression of one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous proteins, is no more than about 40% of the corresponding endogenous protein expression of a reference cell, e.g., a WT CHO cell. In certain embodiments, the expression of one or more endogenous proteins in a cell that has been modified to reduce or eliminate expression of the endogenous proteins, is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding endogenous protein expression of a reference cell, e.g., a WT host cell.

In certain embodiments, the expression of one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous proteins, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous proteins expression of a reference cell, e.g., a WT host cell. In certain embodiments, the expression of one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous proteins, is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding endogenous protein expression of a reference cell, e.g., a WT host cell.

In certain embodiments, the expression of one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in a cell that has been modified to reduce or eliminate expression of the endogenous proteins, is between about 5% and about 40% of the corresponding endogenous protein expression of a reference cell, e.g., a WT host cell.

In certain embodiments, the expression level of the one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in different reference cells (e.g., cells that comprise at least one or both wild-type alleles of the corresponding gene) can vary.

In certain embodiments, a genetic engineering system is employed to reduce or eliminate the expression of one or more particular endogenous protein (e.g., a BAX; BAK; ICAM-1; SIRT-1; MYC; GGTA1; CMAH; LPL; LIPA; LPLA2; BCKDHA; BCKDHB; PPT1; and/or PERK expression). Various genetic engineering systems known in the art can be used for the methods disclosed herein. Non-limiting examples of such systems include the CRISPR/Cas system, the zinc-finger nuclease (ZFN) system, the transcription activator-like effector nuclease (TALEN) system and the use of other tools for reducing or eliminating protein expression by gene silencing, such as small interfering RNAs (siRNAs), short hairpin RNA (shRNA), and microRNA (miRNA).

Any CRISPR/Cas systems known in the art, including traditional, enhanced or modified Cas systems, as well as other bacterial based genome excising tools such as Cpf-1 can be used with the methods disclosed herein.

In certain embodiments, a portion of one or more genes, e.g., genes coding for a endogenous protein such as BAX; BAK; ICAM-1; SIRT-1; MYC; GGTA1; CMAH; LPL; LIPA; LPLA2; BCKDHA; BCKDHB; PPT1; and/or PERK polypeptides, is deleted to reduce or eliminate expression of the corresponding endogenous protein in a host cell. In certain embodiments, at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85% or at least about 90% of the gene is deleted. In certain embodiments, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85% or no more than about 90% of the gene is deleted. In certain embodiments, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of the gene is deleted.

In certain embodiments, at least one exon of a gene encoding a BAX; BAK; ICAM-1; SIRT-1; MYC; GGTA1; CMAH; LPL; LIPA; LPLA2; BCKDHA; BCKDHB; PPT1; and/or PERK polypeptide is at least partially deleted in a host cell. “Partially deleted,” as used herein, refers to at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, no more than about 2%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, no more than about 65%, no more than about 70%, no more than about 75%, no more than about 80%, no more than about 85%, no more than about 90%, no more than about 95%, between about 2% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 2% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 2% and about 70%, between about 10% and about 70%, between about 20% and about 70%, between about 30% and about 70%, between about 40% and about 70%, between about 50% and about 70%, between about 60% and about 70%, between about 65% and about 70%, between about 2% and about 60%, between about 10% and about 60%, between about 20% and about 60%, between about 30% and about 60%, between about 40% and about 60%, between about 50% and about 60%, between about 55% and about 60%, between about 2% and about 50%, between about 10% and about 50%, between about 20% and about 50%, between about 30% and about 50%, between about 40% and about 50%, between about 45% and about 50%, between about 2% and about 40%, between about 10% and about 40%, between about 20% and about 40%, between about 30% and about 40%, between about 35% and about 40%, between about 2% and about 30%, between about 10% and about 30%, between about 20% and about 30%, between about 25% and about 30%, between about 2% and about 20%, between about 5% and about 20%, between about 10% and about 20%, between about 15% and about 20%, between about 2% and about 10%, between about 5% and about 10%, or between about 2% and about 5% of a region, e.g., of the exon, is deleted.

In certain non-limiting embodiments, a CRISPR/Cas9 system is employed to reduce or eliminate the expression of one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide in a host cell. A clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing).

CRISPR/Cas9 strategies can employ a vector to transfect the CHO cell. The guide RNA (gRNA) can be designed for each application as this is the sequence that Cas9 uses to identify and directly bind to the target DNA in a CHO cell. Multiple crRNAs and the tracrRNA can be packaged together to form a single-guide RNA (sgRNA). The sgRNA can be joined together with the Cas9 gene and made into a vector in order to be transfected into CHO cells.

In certain embodiments, the CRISPR/Cas9 system for use in reducing or eliminating the expression of one or more endogenous proteins, e.g., a BAX; BAK; ICAM-1; SIRT-1; MYC; GGTA1; CMAH; LPL; LIPA; LPLA2; BCKDHA; BCKDHB; PPT1; and/or PERK polypeptide, comprises a Cas9 molecule and one or more gRNAs comprising a targeting domain that is complementary to a target sequence of the gene encoding the endogenous protein or a component thereof. In certain embodiments, the target gene is a region of the gene coding for the endogenous product, e.g., a BAX; BAK; ICAM-1; SIRT-1; MYC; GGTA1; CMAH; LPL; LIPA; LPLA2; BCKDHA; BCKDHB; PPT1; and/or PERK polypeptide. The target sequence can be any exon or intron region within the gene.

In certain embodiments, the gRNAs are administered to the CHO cell in a single vector and the Cas9 molecule is administered to the host cell in a second vector. In certain embodiments, the gRNAs and the Cas9 molecule are administered to the host cell in a single vector. Alternatively, each of the gRNAs and Cas9 molecule can be administered by separate vectors. In certain embodiments, the CRISPR/Cas9 system can be delivered to the host cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell). In certain embodiments, administering the CRISPR/Cas9 system to the host cell results in the reduction or elimination of the expression of an endogenous product, e.g., a BAX; BAK; ICAM-1; SIRT-1; MYC; GGTA1; CMAH; LPL; LIPA; LPLA2; BCKDHA; BCKDHB; PPT1; and/or PERK polypeptide.

Using CRISPR/Cas9, a particular target gene can be targeted at one, two, three or more different sites. For example, but not by way of limitation, three different sites within the coding sequence can be targeted using three different gRNAs at the same time using multiplexed ribonucleoprotein delivery. In certain embodiments, multiplexed ribonucleoprotein delivery shows higher gene-editing efficacy and specificity compared to the common plasmid based CRISPR/Cas9 editing. In certain embodiments, double-strand breaks at the gene target site(s) induce indel formations. In certain embodiments, e.g., when multiple sites are targeted due to multiplexed gRNA usage, deletions of sequences between the target sites, e.g., intervening exons, result a frameshift of the CDS of the target protein.

In certain embodiments, sequencing of the PCR-amplified gene locus in the modified cell pools will reveal an interruption of the sequencing reaction at the first gRNA site showing successful targeting for the gene. In certain embodiments the cell pools will comprise modification(s) at all targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n−1” of the “n” targeted genes (where “n” is the number of targeted genes) in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n−2” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n−3” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at “n−4” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool. In certain embodiments the cell pools will comprise modification(s) at one to “n” of the “n” targeted genes in at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the cells in the pool.

In certain embodiments, the genetic engineering system is a ZFN system for reducing or eliminating the expression of one or more particular endogenous protein in a CHO cell, e.g., a BAX; BAK; ICAM-1; SIRT-1; MYC; GGTA1; CMAH; LPL; LIPA; LPLA2; BCKDHA; BCKDHB; PPT1; and/or PERK polypeptide. The ZFN can act as restriction enzyme, which is generated by combining a zinc finger DNA-binding domain with a DNA-cleavage domain. A zinc finger domain can be engineered to target specific DNA sequences which allows the zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the type IIs restriction endonuclease FokI. ZFN modulates the expression of proteins by producing double-strand breaks (DSBs) in the target DNA sequence, which will, in the absence of a homologous template, be repaired by non-homologous end-joining (NHEJ). Such repair can result in deletion or insertion of base-pairs, producing frameshift and preventing the production of the harmful protein (Durai et al., Nucleic Acids Res.; 33 (18): 5978-90 (2005)). Multiple pairs of ZFNs can also be used to completely remove entire large segments of genomic sequence (Lee et al., Genome Res.; 20 (1): 81-9 (2010)).

In certain embodiments, the genetic engineering system is a TALEN system for reducing or eliminating the expression of one or more particular endogenous product, e.g., a BAX; BAK; ICAM-1; SIRT-1; MYC; GGTA1; CMAH; LPL; LIPA; LPLA2; BCKDHA; BCKDHB; PPT1; and/or PERK polypeptide, in a CHO cell. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6 (2011)). In certain embodiments, the target gene encodes a BAX; BAK; ICAM-1; SIRT-1; MYC; GGTA1; CMAH; LPL; LIPA; LPLA2; BCKDHA; BCKDHB; PPT1; and/or PERK.

In certain embodiments, the expression of one or more particular endogenous product, e.g., a BAX; BAK; ICAM-1; SIRT-1; MYC; GGTA1; CMAH; LPL; LIPA; LPLA2; BCKDHA; BCKDHB; PPT1; and/or PERK polypeptide, can be reduced or eliminated using oligonucleotides that have complementary sequences to corresponding nucleic acids (e.g., mRNA). Non-limiting examples of such oligonucleotides include small interference RNA (siRNA), short hairpin RNA (shRNA), and microRNA (miRNA). In certain embodiments, such oligonucleotides can be homologous to at least a portion of a BAX; BAK; ICAM-1; SIRT-1; MYC; GGTA1; CMAH; LPL; LIPA; LPLA2; BCKDHA; BCKDHB; PPT1; and/or PERK nucleic acid sequence, wherein the homology of the portion relative to the corresponding nucleic acid sequence is at least about 75 or at least about 80 or at least about 85 or at least about 90 or at least about 95 or at least about 98 percent. In certain non-limiting embodiments, the complementary portion can constitute at least 10 nucleotides or at least 15 nucleotides or at least 20 nucleotides or at least 25 nucleotides or at least 30 nucleotides and the antisense nucleic acid, shRNA, mRNA or siRNA molecules can be up to 15 or up to 20 or up to 25 or up to 30 or up to 35 or up to 40 or up to 45 or up to 50 or up to 75 or up to 100 nucleotides in length. Antisense nucleic acid, shRNA, mRNA or siRNA molecules can comprise DNA or atypical or non-naturally occurring residues, for example, but not limited to, phosphorothioate residues.

The genetic engineering systems disclosed herein can be delivered into the CHO cell using a viral vector, e.g., retroviral vectors such as gammaretroviral vectors, and lentiviral vectors. Combinations of retroviral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD 114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify the CHO cells disclosed herein. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic engineering of the CHO cell disclosed herein. For example, a nucleic acid molecule can be introduced into the CHO cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation and protoplast fusion. Liposomes can also be potentially beneficial for delivery of nucleic acid molecules into a CHO cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) is injected into a targeted tissue or are injected systemically.

### 4. Integration of Exogenous Nucleic Acids

An exogenous nucleotide sequence, as used herein, is a nucleotide sequence that does not originate from a host cell but can be introduced into a host cell by traditional DNA delivery methods, e.g., by transfection, electroporation, or transformation methods. In certain embodiments, the exogenous nucleotide sequence is a sequence of interest (SOI), e.g., a nucleotide sequence encoding a polypeptide of interest. In certain embodiments, however, the exogenous nucleotide sequences employed in the context of the instant disclosure comprises elements, e.g., one or more recombination recognition sequences (RRs) and one or more selection markers, which facilitate the introduction of additional nucleic acid sequences, e.g., SOIs. In certain embodiments, the exogenous nucleotide sequences facilitating the introduction of additional nucleic acid sequences are referred to herein as “landing pads.” Accordingly, in certain embodiments, a TI host cell can comprise: (1) an exogenous nucleotide sequence that includes one or more SOIs, e.g., an SOI incorporated into a particular locus in a host cell genome via an exogenous site-specific nuclease mediated (e.g., CRISPR/Cas9-mediated) targeted integration; (2) an exogenous nucleotide sequence that includes one or more landing pads; or (3) an exogenous nucleotide sequence that includes one or more landing pads into which one or more SOIs have been incorporated.

In certain embodiments, a TI host cell comprises at least one exogenous nucleotide sequence integrated at one or more integration sites in the genome of the TI host cell. In certain embodiments, the exogenous nucleotide sequence is integrated at one or more integration sites within a specific a locus of the genome of the TI host cell.

### 4.1 Landing Pads

In certain embodiments, an integrated exogenous nucleotide sequence comprises one or more recombination recognition sequence (RRS), wherein the RRS can be recognized by a recombinase. In certain embodiments, the integrated exogenous nucleotide sequence comprises at least two RRSs. In certain embodiments, the integrated exogenous nucleotide sequence comprises two RRSs and the two RRSs are the same. In certain embodiments, the integrated exogenous nucleotide sequence comprises two RRSs and the two RRSs are heterospecific, i.e., not recognized by the same recombinase. In certain embodiments, an integrated exogenous nucleotide sequence comprises three RRSs, wherein the third RRS is located between the first and the second RRS. In certain embodiments, the first and the second RRS are the same and the third RRS is different from the first or the second RRS. In certain embodiments, all three RRSs are heterospecific. In certain embodiments, an integrated exogenous nucleotide sequence comprises four, five, six, seven, or eight RRSs. In certain embodiments, an integrated exogenous nucleotide sequence comprises multiple RRSs. In certain embodiments, the multiple two or more RRSs are the same. In certain embodiments, the two or more RRSs are heterospecific. In certain embodiments each RRS can be recognized by a distinct recombinase. In certain embodiments, the subset of the total number of RRSs are the homospecific, i.e., recognized by the same recombinase, and a subset of the total number of RRSs are heterospecific, i.e., not recognized by the same recombinase. In certain embodiments, the RRS or RRSs can be selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence.

In certain embodiments, the integrated exogenous nucleotide sequence comprises at least one selection marker. In certain embodiments, the integrated exogenous nucleotide sequence comprises one RRS and at least one selection marker. In certain embodiments, the integrated exogenous nucleotide sequence comprises a first and a second RRS, and at least one selection marker. In certain embodiments, a selection marker is located between the first and the second RRS. In certain embodiments, two RRSs flank at least one selection marker, i.e., a first RRS is located 5′ upstream and a second RRS is located 3′ downstream of the selection marker. In certain embodiments, a first RRS is adjacent to the 5′ end of the selection marker and a second RRS is adjacent to the 3′ end of the selection marker.

In certain embodiments, a selection marker is located between a first and a second RRS and the two flanking RRSs are the same. In certain embodiments, the two RRSs flanking the selection marker are both LoxP sequences. In certain embodiments, the two RRSs flanking the selection marker are both FRT sequences. In certain embodiments, a selection marker is located between a first and a second RRS and the two flanking RRSs are heterospecific. In certain embodiments, the first flanking RRS is a LoxP L3 sequence and the second flanking RRS is a LoxP 2L sequence. In certain embodiments, a LoxP L3 sequenced is located 5′ of the selection marker and a LoxP 2L sequence is located 3′ of the selection marker. In certain embodiments, the first flanking RRS is a wild-type FRT sequence and the second flanking RRS is a mutant FRT sequence. In certain embodiments, the first flanking RRS is a Bxb1 attP sequence and the second flanking RRS is a Bxb1 attB sequence. In certain embodiments, the first flanking RRS is a φC31 attP sequence and the second flanking RRS is a φC31 attB sequence. In certain embodiments, the two RRSs are positioned in the same orientation. In certain embodiments, the two RRSs are both in the forward or reverse orientation. In certain embodiments, the two RRSs are positioned in opposite orientation.

In certain embodiments, a selection marker can be an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, or mycophenolic acid. In certain embodiments, a selection marker can be a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, or a T-Sapphire marker. In certain embodiments, the selection marker can be a fusion construct comprising at least two selection markers. In certain embodiments the gene encoding a selection marker or a fragment of the selection marker can be fused to the gene encoding a different selection marker or a fragment thereof.

In certain embodiments, the integrated exogenous nucleotide sequence comprises two selection markers flanked by two RRSs, wherein a first selection marker is different from a second selection marker. In certain embodiments, the two selection markers are both selected from the group consisting of a glutamine synthetase selection marker, a thymidine kinase selection marker, a HYG selection marker, and a puromycin resistance selection marker. In certain embodiments, the integrated exogenous nucleotide sequence comprises a thymidine kinase selection marker and a HYG selection marker. In certain embodiments, the first selection maker is selected from the group consisting of an aminoglycoside phosphotransferase (APH) (e.g., hygromycin phosphotransferase (HYG), neomycin and G418 APH), dihydrofolate reductase (DHFR), thymidine kinase (TK), glutamine synthetase (GS), asparagine synthetase, tryptophan synthetase (indole), histidinol dehydrogenase (histidinol D), and genes encoding resistance to puromycin, blasticidin, bleomycin, phleomycin, chloramphenicol, Zeocin, and mycophenolic acid, and the second selection maker is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire marker. In certain embodiments, the first selection marker is a glutamine synthetase selection marker and the second selection marker is a GFP marker. In certain embodiments, the two RRSs flanking both selection markers are the same. In certain embodiments, the two RRSs flanking both selection markers are different.

In certain embodiments, the selection marker is operably linked to a promoter sequence. In certain embodiments, the selection marker is operably linked to an SV40 promoter. In certain embodiments, the selection marker is operably linked to a Cytomegalovirus (CMV) promoter.

In certain embodiments, the integrated exogenous nucleotide sequence comprises at least one selection marker and an IRES, wherein the IRES is operably linked to the selection marker. In certain embodiments, the selection marker operably linked to the IRES is selected from the group consisting of a GFP, an eGFP, a synthetic GFP, a YFP, an eYFP, a CFP, an mPlum, an mCherry, a tdTomato, an mStrawberry, a J-red, a DsRed-monomer, an mOrange, an mKO, an mCitrine, a Venus, a YPet, an Emerald, a CyPet, an mCFPm, a Cerulean, and a T-Sapphire marker. In certain embodiments, the selection marker operably linked to the IRES is a GFP marker. In certain embodiments, the integrated exogenous nucleotide sequence comprises an IRES and two selection markers flanked by two RRSs, wherein the IRES is operably linked to the second selection marker.

In certain embodiments, the integrated exogenous nucleotide sequence comprises an IRES and three selection markers flanked by two RRSs, wherein the IRES is operably linked to the third selection marker. In certain embodiments, the integrated exogenous nucleotide sequence comprises an IRES and three selection markers flanked by two RRSs, wherein the IRES is operably linked to the third selection marker. In certain embodiments, the third selection marker is different from the first or the second selection marker. In certain embodiments, the integrated exogenous nucleotide sequence comprises a first selection marker operably linked to a promoter and a second selection marker operably linked to an IRES. In certain embodiments, the integrated exogenous nucleotide sequence comprises a glutamine synthetase selection marker operably linked to a SV40 promoter and a GFP selection marker operably linked to an IRES. In certain embodiments, the integrated exogenous nucleotide sequence comprises a thymidine kinase selection marker and a HYG selection marker operably linked to a CMV promoter and a GFP selection marker operably linked to an IRES.

In certain embodiments, the integrated exogenous nucleotide sequence comprises three RRSs. In certain embodiments, the third RRS is located between the first and the second RRS. In certain embodiments, all three RRSs are the same. In certain embodiments, the first and the second RRS are the same, and the third RRS is different from the first or the second RRS. In certain embodiments, all three RRSs are heterospecific.

### 4.2 Sequences of Interest (SOIs)

In certain embodiments, the integrated exogenous nucleotide sequence comprises at least one exogenous SOI. In certain embodiments, the integrated exogenous nucleotide sequence comprises at least one selection marker and at least one exogenous SOI. In certain embodiments, the integrated exogenous nucleotide sequence comprises at least one selection marker, at least one exogenous SOI, and at least one RRS. In certain embodiments, the integrated exogenous nucleotide sequence comprises at least one, at least two, at least three, at least four, at least five, at least six, at least seven, at least eight or more SOIs. In certain embodiments the SOIs are the same. In certain embodiments, the SOIs are different.

In certain embodiments the SOI encodes a single chain antibody or fragment thereof. In certain embodiments, the SOI encodes an antibody heavy chain sequence or fragment thereof. In certain embodiments, the SOI encodes an antibody light chain sequence or fragment thereof. In certain embodiments, an integrated exogenous nucleotide sequence comprises an SOI encoding an antibody heavy chain sequence or fragment thereof and an SOI encoding an antibody light chain sequence or fragment thereof. In certain embodiments, an integrated exogenous nucleotide sequence comprises an SOI encoding a first antibody heavy chain sequence or fragment thereof, an SOI encoding a second antibody heavy chain sequence or fragment thereof, and an SOI encoding an antibody light chain sequence or fragment thereof. In certain embodiments, an integrated exogenous nucleotide sequence comprises an SOI encoding a first antibody heavy chain sequence or fragment thereof, an SOI encoding a second antibody heavy chain sequence or fragment thereof, an SOI encoding a first antibody light chain sequence or fragment thereof and a second SOI encoding an antibody light chain sequence or fragment thereof. In certain embodiments, the number of SOIs encoding for heavy and light chain sequences can be selected to achieve a desired expression level of the heavy and light chain polypeptides, e.g., to achieve a desired amount of bispecific antibody production. In certain embodiments, the individual SOIs encoding heavy and light chain sequences can be integrated, e.g., into a single exogenous nucleic acid sequence present at a single integration site, into multiple exogenous nucleic acid sequences present at a single integration site, or into multiple exogenous nucleic acid sequences integrated at distinct integration sites within the TI host cell.

In certain embodiments, the integrated exogenous nucleotide sequence comprises at least one selection marker, at least one exogenous SOI, and one RRS. In certain embodiments, the RRS is located adjacent to at least one selection marker or at least one exogenous SOI. In certain embodiments, the integrated exogenous nucleotide sequence comprises at least one selection marker, at least one exogenous SOI, and two RRSs. In certain embodiments, the integrated exogenous nucleotide sequence comprises at least one selection marker and at least one exogenous SOI located between the first and the second RRS. In certain embodiments, the two RRSs flanking the selection marker and the exogenous SOI are the same. In certain embodiments, the two RRSs flanking the selection marker and the exogenous SOI are different. In certain embodiments, the first flanking RRS is a LoxP L3 sequence and the second flanking RRS is a LoxP 2L sequence. In certain embodiments, a L3 LoxP sequenced is located 5′ of the selection marker and the exogenous SOI, and a LoxP 2L sequence is located 3′ of the selection marker and the exogenous SOI.

In certain embodiments, the integrated exogenous nucleotide sequence comprises three RRSs and two exogenous SOIs, and the third RRS is located between the first and the second RRS. In certain embodiments, the first SOI is located between the first and the third RRS, and the second SOI is located between the third and the second RRS. In certain embodiments, the first and the second SOI are different. In certain embodiments, the first and the second RRS are the same and the third RRS is different from the first or the second RRS. In certain embodiments, all three RRSs are heterospecific. In certain embodiments, the first RRS is a LoxP L3 site, the second RRS is a LoxP 2L site, and the third RRS is a LoxFas site. In certain embodiments, the integrated exogenous nucleotide sequence comprises three RRSs, one exogenous SOI, and one selection marker. In certain embodiments, the SOI is located between the first and the third RRS, and the selection marker is located between the third and the second RRS. In certain embodiments, the integrated exogenous nucleotide sequence comprises three RRSs, two exogenous SOIs, and one selection marker. In certain embodiments, the first SOI and the selection marker are located between the first and the third RRS, and the second SOI is located between the third and the second RRS.

In certain embodiments, the exogenous SOI encodes a polypeptide of interest. Such polypeptides of interest can be selected from the group including, but not limited to, an antibody, an enzyme, a cytokine, a growth factor, a hormone, a viral protein, a bacterial protein, a vaccine protein, or a protein with therapeutic function. In certain embodiments, the exogenous SOI encodes an antibody or an antigen-binding fragment thereof. In certain embodiments, the exogenous SOI encodes a single chain antibody, an antibody light chain, an antibody heavy chain, a single-chain Fv fragment (scFv), or an Fc fusion protein. In certain embodiments, the exogenous SOI (or SOIs) encodes a standard antibody. In certain embodiments, the exogenous SOI (or SOIs) encodes a half-antibody, for example, but not limited to, antibodies B, Q, T and mAb I of the present disclosure. In certain embodiments, the exogenous SOI (or SOIs) encodes a complex antibody. In certain embodiments, the complex antibody can be a bispecific antibody, for example, but not limited to, Bispecific Molecule A, Bispecific Molecule B, Bispecific Molecule C, or Bispecific Molecule D of the present disclosure. In certain embodiments, the exogenous SOI is operably linked to at least one cis-acting element, for example, a promoter or an enhancer. In certain embodiments, the exogenous SOI is operably linked to a CMV promoter.

In certain embodiments, the integrated exogenous nucleotide sequence comprises two RRSs and at least two exogenous SOIs located between the two RRSs. In certain embodiments, SOIs encoding one heavy chain and one light chain of an antibody are located between the two RRSs. In certain embodiments, SOIs encoding one heavy chain and two light chains of an antibody are located between the two RRSs. In certain embodiments, SOIs encoding different combinations of copies of heavy chain and light chain of an antibody are located between the two RRSs.

In certain embodiments, the integrated exogenous nucleotide sequence comprises three RRSs and at least two exogenous SOIs, and the third RRS is located between the first and the second RRS. In certain embodiments, at least one SOI is located between the first and the third RRS, and at least one SOI is located between the third and the second RRS. In certain embodiments, the first and the second RRS are the same and the third RRS is different from the first or the second RRS. In certain embodiments, all three RRSs are heterospecific. In certain embodiments, SOIs encoding one heavy chain and one light chain of a first antibody are located between the first and the third RRS, and SOIs encoding one heavy chain and one light chain of a second antibody are located between the third and the second RRS. In certain embodiments, SOIs encoding one heavy chain and two light chains of a first antibody are located between the first and the third RRS, and SOIs encoding one heavy chain and one light chain of a second antibody are located between the third RRS and the second RRS. In certain embodiments, SOIs encoding one heavy chain and three light chains of a first antibody are located between the first and the third RRS, and SOIs encoding one light chain of the first antibody and one heavy chain and one light chain of a second antibody are located between the third RRS and the second RRS. In certain embodiments, SOIs encoding one heavy chain and three light chains of a first antibody are located between the first and the third RRS, and SOIs encoding two light chains of the first antibody and one heavy chain and one light chain of a second antibody are located between the third RRS and the second RRS. In certain embodiments, SOIs encoding different combinations of copies of heavy chains and light chains of multiple antibodies are located between the first and the third RRS, and between the third and the second RRS.

In certain embodiments, the number of SOIs is selected to increase the titer and/or specific productivity of the host cells expressing the SOIs. For example, but not by way of limitation, the incorporation of two, three, four, five, six, seven, eight, or more SOIs can result in increased titer and/or specific productivity.

In the context of antibody expression, the inclusion of an additional heavy or light chain encoding SOIs can result in increased titer and/or specific productivity. For example, but not by way of limitation, when increasing copy number from one heavy chain and one light chain (HL) to one heavy chain and two light chain encoding sequences (HLL), an increase in titer and/or specific productivity can be achieved. Similarly, as outlined in the examples below, increasing from HLL (three SOIs) to HLL-HL (five SOIs) or HLL-HLL (six SOIs) can provide for an increase in titer and/or specific productivity. Additionally, increasing copy number to HLL-HL (five SOIs) or HLL-HLHL (seven SOIs) can provide for an increase in titer and/or specific productivity. Additional options for heavy and light chain SOI copy numbers include, but are not limited to HHL; HHL-H; HLL-H; HHL-HH; HHL-HL; HHL-LL; HLL-HH; HLL-HL; HLL-LL; HHL-HHL; HHL-HHH; HHL-HLL; HIIL-LLL; HLL-HHL; HLL-HHH; HLL-LLL; HHL-HIHL; HHL-HHHIH; HHL-HHLL; HHL-HLLL; HHL-LLLL; HLL-HIHL; HLL-HHHH; HLL-HLLL; and HLL-LLLL. In certain embodiments, the inclusion of additional copies occurs at a single genomic locus, while in certain embodiments the SOI copies can be integrated at two or more loci, e.g., multiple copies can be integrated at a single locus and one or more copies integrated at one or more additional loci.

In certain embodiments, the position of the SOIs, e.g., whether one SOI is located 3′ or 5′ relative to another SOI, is selected to increase the titer and/or specific productivity of the host cells expressing the SOIs. For example, but not by way of limitation, in the context of antibody production, the integrated position of heavy and light chain SOIs can result in increased titer and/or specific productivity. In certain embodiments, the relative position of heavy and light chain SOIs can impact the titer and specific productivity, despite no change in SOI copy number

In certain embodiments, targeted integration can be combined with transposon-mediated genomic integration. In certain embodiments, the targeted integration can be followed by transposon-mediated genomic integration. In certain embodiments, the targeted integration can be performed concurrently with transposon-mediated genomic integration. In certain embodiments, the targeted integration can be followed by transposon-mediated genomic integration.

### 4.3. Targeted Integration via Recombinase-Mediated Recombination

A “recombination recognition sequence” (RRS) is a nucleotide sequence recognized by a recombinase and is necessary and sufficient for recombinase-mediated recombination events. A RRS can be used to define the position where a recombination event will occur in a nucleotide sequence.

In certain embodiments, a RRS is selected from the group consisting of a LoxP sequence, a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, a Lox66 sequence, a FRT sequence, a Bxb1 attP sequence, a Bxb1 attB sequence, a φC31 attP sequence, and a φC31 attB sequence.

In certain embodiments, a RRS can be recognized by a Cre recombinase. In certain embodiments, a RRS can be recognized by a FLP recombinase. In certain embodiments, a RRS can be recognized by a Bxb1 integrase. In certain embodiments, a RRS can be recognized by a φC31 integrase.

In certain embodiments when the RRS is a LoxP site, the host cell requires the Cre recombinase to perform the recombination. In certain embodiments when the RRS is a FRT site, the host cell requires the FLP recombinase to perform the recombination. In certain embodiments when the RRS is a Bxb1 attP or a Bxb1 attB site, the host cell requires the Bxb1 integrase to perform the recombination. In certain embodiments when the RRS is a φC31 attP or a φC31attB site, the host cell requires the φC31 integrase to perform the recombination. The recombinases can be introduced into a host cell using an expression vector comprising coding sequences of the enzymes.

The Cre-LoxP site-specific recombination system has been widely used in many biological experimental systems. Cre is a 38-kDa site-specific DNA recombinase that recognizes 34 bp LoxP sequences. Cre is derived from bacteriophase P1 and belongs to the tyrosine family site-specific recombinase. Cre recombinase can mediate both intra and intermolecular recombination between LoxP sequences. The LoxP sequence is composed of an 8 bp nonpalindromic core region flanked by two 13 bp inverted repeats. Cre recombinase binds to the 13 bp repeat thereby mediating recombination within the 8 bp core region. Cre-LoxP-mediated recombination occurs at a high efficiency and does not require any other host factors. If two LoxP sequences are placed in the same orientation on the same nucleotide sequence, Cre-mediated recombination will excise DNA sequences located between the two LoxP sequences as a covalently closed circle. If two LoxP sequences are placed in an inverted position on the same nucleotide sequence, Cre-mediated recombination will invert the orientation of the DNA sequences located between the two sequences. LoxP sequences can also be placed on different chromosomes to facilitate recombination between different chromosomes. If two LoxP sequences are on two different DNA molecules and if one DNA molecule is circular, Cre-mediated recombination will result in integration of the circular DNA sequence.

In certain embodiments, a LoxP sequence is a wild-type LoxP sequence. In certain embodiments, a LoxP sequence is a mutant LoxP sequence. Mutant LoxP sequences have been developed to increase the efficiency of Cre-mediated integration or replacement. In certain embodiments, a mutant LoxP sequence is selected from the group consisting of a LoxP L3 sequence, a LoxP 2L sequence, a LoxFas sequence, a Lox511 sequence, a Lox2272 sequence, a Lox2372 sequence, a Lox5171 sequence, a Loxm2 sequence, a Lox71 sequence, and a Lox66 sequence. For example, the Lox71 sequence has 5 bp mutated in the left 13 bp repeat. The Lox66 sequence has 5 bp mutated in the right 13 bp repeat. Both the wild-type and the mutant LoxP sequences can mediate Cre-dependent recombination.

The FLP-FRT site-specific recombination system is similar to the Cre-Lox system. It involves the flippase (FLP) recombinase, which is derived from the 2 μm plasmid of the yeast Saccharomyces cerevisiae. FLP also belongs to the tyrosine family site-specific recombinase. The FRT sequence is a 34 bp sequence that consists of two palindromic sequences of 13 bp each flanking an 8 bp spacer. FLP binds to the 13 bp palindromic sequences and mediates DNA break, exchange and ligation within the 8 bp spacer. Similar to the Cre recombinase, the position and orientation of the two FRT sequences determine the outcome of FLP-mediated recombination. In certain embodiments, a FRT sequence is a wild-type FRT sequence. In certain embodiments, a FRT sequence is a mutant FRT sequence. Both the wild-type and the mutant FRT sequences can mediate FLP-dependent recombination. In certain embodiments, a FRT sequence is fused to a responsive receptor domain sequence, such as, but not limited to, a tamoxifen responsive receptor domain sequence.

Bxb1 and φC31 belong to the serine recombinase family. They are both derived from bacteriophages and are used by these bacteriophages to establish lysogeny to facilitate site-specific integration of the phage genome into the bacterial genome. These integrases catalyze site-specific recombination events between short (40-60 bp) DNA substrates termed attP and attB sequences that are originally attachment sites located on the phage DNA and bacterial DNA, respectively. After recombination, two new sequences are formed, which are termed attL and attR sequences and each contains half sequences derived from attP and attB. Recombination can also occur between attL and attR sequences to excise the integrated phage out of the bacterial DNA. Both integrases can catalyze the recombination without the aid of any additional host factors. In the absence of any accessory factors, these integrases mediate unidirectional recombination between attP and attB with greater than 80% efficiency. Because of the short DNA sequences that can be recognized by these integrases and the unidirectional recombination, these recombination systems have been developed as a complement to the widely-used Cre-LoxP and FRT-FLP systems for genetic engineering purposes.

The terms “matching RRSs” and “homospecific RRSs” indicates that a recombination occurs between two RRSs. In certain embodiments, the two matching RRSs are the same. In certain embodiments, both RRSs are wild-type LoxP sequences. In certain embodiments, both RRSs are mutant LoxP sequences. In certain embodiments, both RRSs are wild-type FRT sequences. In certain embodiments, both RRSs are mutant FRT sequences. In certain embodiments, the two matching RRSs are different sequences but can be recognized by the same recombinase. In certain embodiments, the first matching RRS is a Bxb1 attP sequence and the second matching RRS is a Bxb1 attB sequence. In certain embodiments, the first matching RRS is a φC31 attB sequence and the second matching RRS is a φC31 attB sequence.

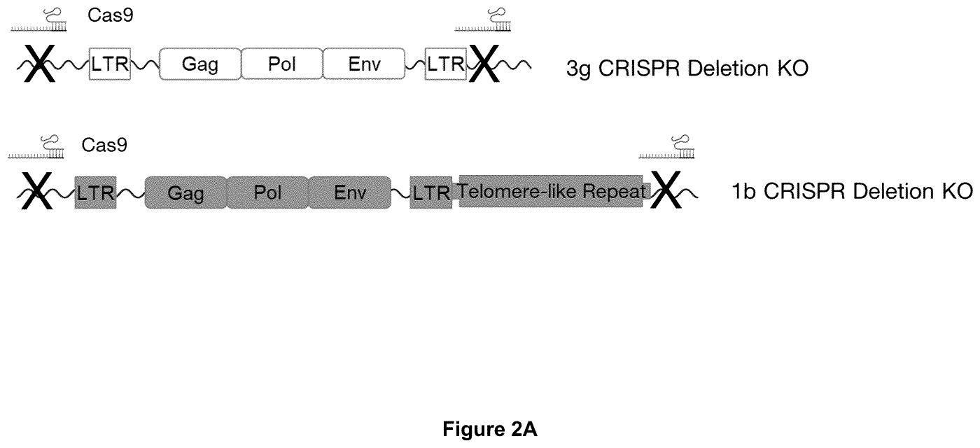

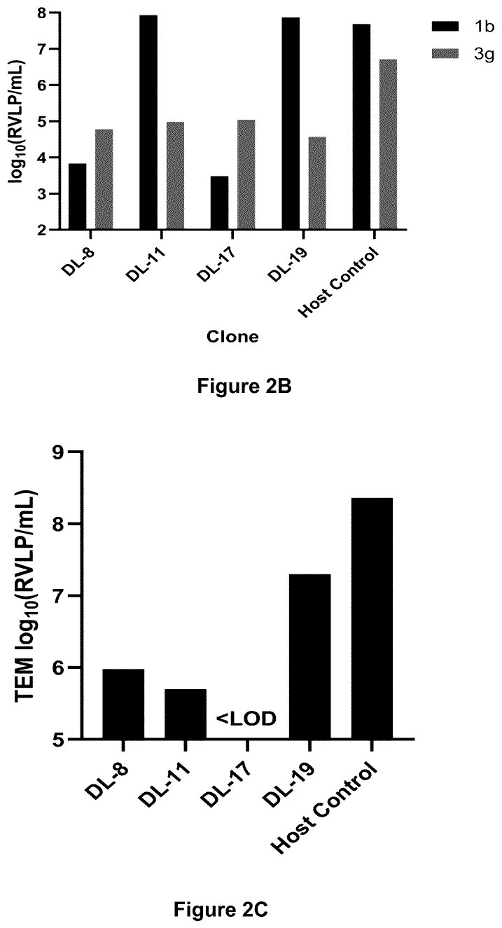

In certain embodiments, an integrated exogenous nucleotide sequence comprises two RRSs and a vector comprises two RRSs matching the two RRSs on the integrated exogenous nucleotide sequence, i.e., the first RRS on the integrated exogenous nucleotide sequence matches the first RRS on the vector and the second RRS on the integrated exogenous nucleotide sequence matches the second RRS on the vector. In certain embodiments, the first RRS on the integrated exogenous nucleotide sequence and the first RRS on the vector are the same as the second RRS on the integrated exogenous nucleotide sequence and the second RRS on the vector. A non-limiting example of such a “single-vector RMCE” strategy is presented in FIG. 2A of PCT Application PCT/US2018/067070 (Publication No. WO2019126634). In certain embodiments, the first RRS on the integrated exogenous nucleotide sequence and the first RRS on the vector are different from the second RRS on the integrated exogenous nucleotide sequence and the second RRS on the vector. In certain embodiments, the first RRS on the integrated exogenous nucleotide sequence and the first RRS on the vector are both LoxP L3 sequences, and the second RRS on the integrated exogenous nucleotide sequence and the second RRS on the vector are both LoxP 2L sequences.

In certain embodiments, a “two-vector RMCE” strategy is employed. For example, but not by way of limitation, an integrated exogenous nucleotide sequence could comprise three RRSs, e.g., an arrangement where the third RRS (“RRS3”) is present between the first RRS (“RRS1”) and the second RRS (“RRS2”), while a first vector comprises two RRSs matching the first and the third RRS on the integrated exogenous nucleotide sequence, and a second vector comprises two RRSs matching the third and the second RRS on the integrated exogenous nucleotide sequence.

An example of a two vector RMCE strategy is illustrated in FIG. 4 PCT Application PCT/US2018/067070 (Publication No. WO2019126634). In such an example, RRS1, RRS2, and RRS3 are heterospecific, e.g., they do not cross-react with each other. In some embodiments, one vector (front) comprises the RRS1, a first SOI and a promoter followed by a start codon and RRS3 (in this order). The other vector (back) comprises the RRS3 fused to the coding sequence of a marker without the start codon (ATG), an SOI 2 and the RRS2 (in this order). Additional nucleotides may be inserted between the RRS3 site and the selection marker sequence to ensure in frame translation for the fusion protein. In some embodiments, the first SOI encodes an antibody. In some embodiments, the antibody is a single chain antibody, an antibody light chain, an antibody heavy chain, a single-chain Fv fragment (scFv), or an Fc fusion protein. In some embodiments, the second SOI encodes an antibody. In some embodiments, the antibody is a single chain antibody, an antibody light chain, an antibody heavy chain, a single-chain Fv fragment (scFv), or an Fc fusion protein. In certain embodiments the antibodies encoded by the first and second SOIs pair to form a multispecific, e.g., bispecific antibody.

Such two vector RMCE strategies allow for the introduction of eight or more SOIs by incorporating the appropriate number of SOIs between each pair of RRSs.

Both single-vector and two-vector RMCE allow for unidirectional integration of one or more donor DNA molecule(s) into a pre-determined site of a host cell genome, and precise exchange of a DNA cassette present on the donor DNA with a DNA cassette on the host genome where the integration site resides. The DNA cassettes are characterized by two heterospecific RRSs flanking at least one selection marker (although in certain two-vector RMCE examples a “split selection marker” can be used as outlined herein) and/or at least one exogenous SOI. RMCE involves double recombination cross-over events, catalyzed by a recombinase, between the two heterospecific RRSs within the target genomic locus and the donor DNA molecule. RMCE is designed to introduce a copy of the SOI or selection marker into the pre-determined locus of a host cell genome. Unlike recombination which involves just one cross-over event, RMCE can be implemented such that prokaryotic vector sequences are not introduced into the host cell genome, thus reducing and/or preventing unwanted triggering of host immune or defense mechanisms. The RMCE procedure can be repeated with multiple DNA cassettes.

In certain embodiments, targeted integration is achieved by one cross-over recombination event, wherein one exogenous nucleotide sequence comprising one RRS adjacent to at least one exogenous SOI or at least one selection marker is integrated into a pre-determined site of a host cell genome. In certain embodiments, targeted integration is achieved by one RMCE, wherein a DNA cassette comprising at least an exogenous SOI or at least one selection marker flanked by two heterospecific RRSs is integrated into a pre-determined site of a host cell genome. In certain embodiments, targeted integration is achieved by two RMCEs, wherein two different DNA cassettes, each comprising at least an exogenous SOI or at least one selection marker flanked by two heterospecific RRSs, are both integrated into a pre-determined site of a host cell genome. In certain embodiments, targeted integration is achieved by multiple RMCEs, wherein DNA cassettes from multiple vectors, each comprising at least an exogenous SOI or at least one selection marker flanked by two heterospecific RRSs, are all integrated into a pre-determined site of a host cell genome. In certain embodiments the selection marker can be partially encoded on the first the vector and partially encoded on the second vector such that the integration of both RMCEs allows for the expression of the selection marker. An example of such a system is presented in FIG. 4 PCT Application PCT/US2018/067070 (Publication No. WO2019126634).

In certain embodiments, targeted integration via recombinase-mediated recombination leads to a selection marker or one or more exogenous SOI integrated into one or more pre-determined integration sites of a host cell genome along with sequences from a prokaryotic vector.

In certain embodiments, targeted integration via recombinase-mediated recombination leads to selection marker or one or more exogenous SOI integrated into one or more pre-determined integration sites of a host cell genome free of sequences from a prokaryotic vector.

### 4.4 Targeted Integration via Homologous Recombination, HDR, or NHEJ

The presently disclosed subject matter also relates to targeted integration mediated by homologous recombination or by an exogenous site-specific nuclease followed by HDR or NHEJ.

Homologous recombination is a recombination between DNA molecules that share extensive sequence homology. It can be used to direct error-free repair of double-stranded DNA breaks and generates sequence variation in gametes during meiosis. Since homologous recombination involves the exchange of genetic information between two homologous DNA molecules, it does not alter the overall arrangement of the genes on a chromosome. During homologous recombination, a nick or break forms in double-stranded DNA (dsDNA), followed by the invasion of a homologous dsDNA molecule by a single-stranded DNA end, pairing of homologous sequences, branch migration to form a Holliday junction, and final resolution of the Holliday junction.

Double-strand break (DSB) is the most severe form of DNA damage and repair of such DNA damage is essential for the maintenance of genome integrity in all organisms. There are two major repair pathways to repair DSBs. The first repair pathway is homology-directed repair (HDR) pathway and homologous recombination is the most common form of HDR. Since HDR requires the presence of homologous DNA present in the cell, this repair pathway is normally active in S and G2 phase of the cell cycle wherein newly replicated sister chromatids are available as homologous templates. HDR is also a major repair pathway to repair collapsed replication forks during DNA replication. HDR is considered as a relatively error-free repair pathway. The second repair pathway for DSBs is non-homologous end joining (NHEJ). NHEJ is a repair pathway wherein the ends of a broken DNA are ligated together without the requirement for a homologous DNA template.

Targeted integration can be facilitated by exogenous site-specific nucleases followed by HDR. This is due to that the frequency of homologous recombination can be increased by introducing a DSB at a specific target genomic site. In certain embodiments, an exogenous nuclease can be selected from the group consisting of a zinc finger nuclease (ZFN), a ZFN dimer, a transcription activator-like effector nuclease (TALEN), a TAL effector domain fusion protein, an RNA-guided DNA endonuclease, an engineered meganuclease, and a clustered regularly interspaced short palindromic repeats (CRISPR)-associated (Cas) endonuclease.

CRISPR/Cas and TALEN systems are two genome editing tools that offer the best ease of construction and high efficiency. CRISPR/Cas was identified as an immune defense mechanism of bacteria against invading bacteriophages. Cas is a nuclease that, when guided by a synthetic guide RNA (gRNA), is capable of associating with a specific nucleotide sequence in a cell and editing the DNA in or around that nucleotide sequence, for instance by making one or more of a single-strand break, a DSB, and/or a point mutation. TALEN is an engineered site-specific nuclease, which is composed of the DNA-binding domain of TALE (transcription activator-like effectors) and the catalytic domain of restriction endonuclease FokI. By changing the amino acids present in the highly variable residue region of the monomers of the DNA binding domain, different artificial TALENs can be created to target various nucleotides sequences. The DNA binding domain subsequently directs the nuclease to the target sequences and creates a DSB.

Targeted integration via homologous recombination or HIDR involves the presence of homologous sequences to the integration site. In certain embodiments, the homologous sequences are present on a vector. In certain embodiments, the homologous sequences are present on a polynucleotide.

In certain embodiments, a vector for targeted integration of exogenous nucleotide sequences into a host cell comprises nucleotide sequences homologous to an endogenous sequence comprising a portion of the contig sequence of one of the contigs NW_006874047.1, NW_006884592.1, NW_006881296.1, NW_003616412.1, NW_003615063.1, NW_006882936.1, and NW_003615411.1, or to a gene selected from the group consisting of LOC107977062, LOC100768845, ITPR2, ERE67000.1, UBAP2, MTMR2, and XP_003512331.2, or to a sequence selected from SEQ ID Nos. 1-7 flanking at least one selection marker. In certain embodiments, a vector for targeted integration of exogenous nucleotide sequences into a host cell comprises nucleotide sequences homologous to an endogenous sequence comprising a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, and XP_003512331.2, or to a sequence selected from SEQ ID Nos. 1-7 flanking at least one selection marker and at least one exogenous SOI. In certain embodiments, a vector for targeted integration of exogenous nucleotide sequences into a host cell comprises nucleotide sequences at least 50% homologous to a sequence selected from a portion of the contig sequence of one of the contigs NW_006874047.1, NW_006884592.1, NW_006881296.1, NW_003616412.1, NW_003615063.1, NW_006882936.1, and NW_003615411.1, and SEQ ID Nos. 1-7 flanking a DNA cassette, wherein the DNA cassette comprises at least one selection marker and at least one exogenous SOI flanked by two RRSs. In certain embodiments, a vector for targeted integration of exogenous nucleotide sequences into a host cell comprises nucleotide sequences at least 50% homologous to an endogenous a sequence of a portion of the contig sequence of one of the contigs NW_006874047.1, NW_006884592.1, NW_006881296.1, NW_003616412.1, NW_003615063.1, NW_006882936.1, and NW_003615411.1, or to a gene selected from the group consisting of LOC107977062, LOC100768845, ITPR2, ERE67000.1, UBAP2, MTMR2, and XP_003512331.2 flanking a DNA cassette, wherein the DNA cassette comprises at least one selection marker and at least one exogenous SOI flanked by two RRSs. In certain embodiments, the vector nucleotide sequences are at least about 50%, at least about 60%, at least about 70%, at least about 80%, at least about 90%, at least about 95%, at least about 99%, or at least about 99.9% homologous to an endogenous sequence of a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, and XP_003512331.2, or to a sequence selected from SEQ ID Nos. 1-7. In certain embodiments, the vector is selected from the group consisting of an adenovirus vector, an adeno-associated virus vector, a lentivirus vector, a retrovirus vector, an integrating phage vector, a non-viral vector, a transposon and/or transposase vector, an integrase substrate, and a plasmid.

In certain embodiments, a polynucleotide for targeted integration of exogenous nucleotide sequences into a host cell comprises nucleotide sequences homologous to an endogenous sequence of a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, and XP_003512331.2, or to a sequence selected from SEQ ID Nos. 1-7 flanking at least one selection marker. In certain embodiments, a polynucleotide for targeted integration of exogenous nucleotide sequences into a host cell comprises nucleotide sequences homologous to an endogenous sequence of a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, and XP_003512331.2, or to a sequence selected from SEQ ID Nos. 1-7 flanking at least one selection marker and at least one exogenous SOI. In certain embodiments, a polynucleotide for targeted integration of exogenous nucleotide sequences into a host cell comprises nucleotide sequences at least 50% homologous to a sequence selected from SEQ ID Nos. 1-7 flanking a DNA cassette, wherein the DNA cassette comprises at least one selection marker and at least one exogenous SOI flanked by two RRSs. In certain embodiments, a polynucleotide for targeted integration of exogenous nucleotide sequences into a host cell comprises nucleotide sequences at least 50% homologous to an endogenous sequence of a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, and XP_003512331.2 flanking a DNA cassette, wherein the DNA cassette comprises at least one selection marker and at least one exogenous SOI flanked by two RRSs. In certain embodiments, the flanking nucleotide sequences are at least about 50%, at least about 60%, at least about 70%, at least about 80%, at least about 90%, at least about 95%, at least about 99%, or at least about 99.9% homologous to an endogenous sequence of a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, and XP_003512331.2, or to a sequence selected from SEQ ID Nos. 1-7.

In certain embodiments, homologous recombination is carried out without any accessory factors. In certain embodiments, homologous recombination is facilitated by the presence of vectors that are capable of integration. In certain embodiments, an integrating vector is selected from the group consisting of an adeno-associated virus vector, a lentivirus vector, a retrovirus vector, and an integrating phage vector.

### 4.5 Transposon-Mediated Genomic Integration

The presently disclosed subject matter also contemplates, in certain embodiments, transposon-mediated genomic integration of one or more exogenous nucleic acids into a host cell. As outlined herein, in certain embodiments, the targeted integration can be performed concurrently with transposon-mediated genomic integration can be performed concurrently with targeted integration. In certain embodiments, transposon-mediated genomic integration can be followed by targeted integration. In certain embodiments, transposon-mediated genomic integration can be preceded by targeted integration.

Transposons useful in connection with the methods described herein are known in the art and include, but are not limited to, the following:
piggyBac transposons (see, e.g., Wilson et. al., Molecular Therapy, 15(1):139-145 (2007));
sleeping beauty transposons (see, e.g., Ivics et al., Cell 91:501-510 (1997)); and
Tol2 transposons (see, e.g., Balciunas et al., PLoS Genent. November 10; 2 (11):e169. doi: 10.1371/journal.pgen.0020169. Epub 2006 Aug. 28).

In general, the transposons useful in connection with the methods of the instant disclosure translocate via a non-replicative, ‘cut-and-paste’ mechanism. For example, while not being bound by theory, transposition catalyzed by transposons useful in connection with the methods described herein, can proceed by the recognition of two terminal inverted repeats (TIRs) by a DNA transposase that cleaves its target and consequently releases the DNA transposon from its donor sequence (e.g., donor plasmid). Upon excision, the transposons can integrate into the host cell genome cleaved by the same transposase at a corresponding sequence within the genome.

### 4.6 Regulated Expression of SOIs

There are many cases where protein expression levels are not optimal mainly because the encoded proteins are difficult-to-express. The low expression level of difficult-to-express proteins can have diverse and difficult to identify causes. One possibility is the toxicity of the expressed proteins in the host cells. In such cases, a regulated expression system can be used to express toxic proteins where the sequences of interest encoding the proteins are under the control of an inducible promoter. In these systems, expression of the difficult-to-express proteins is only prompted when a regulator, e.g., small molecule, such as, but not limited to, tetracycline or its analogue, doxycycline (DOX), is added to the culture. Regulating the expression of toxic proteins could alleviate the toxic effects, allowing the cultures to achieve the desired cell growth prior to production. In certain embodiments, a regulated expression system comprises at least one SOI that is transcribed under a regulated promoter operably linked thereto. In certain embodiments, regulated expression system can be used to determine the underlying causes of low protein expression for a difficult-to-express molecule, such as, but not limited to, an antibody. In certain embodiments, the ability to selectively turn off the expression of a SOI in a regulated expression system can be used to link expression of a SOI to an observed adverse effect.

In certain embodiments, to minimize transcriptional and cell line variability effects during the root cause analysis of difficult-to-express molecules, a regulated expression system can be used. For example, but not by way of limitation, the expression of the SOI can be triggered by addition to the culture of a regulator, e.g., doxycycline. In certain embodiments, the regulated expression vector utilizes a tetracycline-regulated promoter to express the SOI, allowing for regulated expression of the SOI.

In certain embodiments, the regulated expression system described in the present disclosure can be used to successfully determine the underlying cause(s) of low protein expression of an SOI, e.g., a therapeutic antibody, as compared to control cell line. In certain embodiments, once the lower relative expression of a SOI, e.g., a therapeutic antibody, in a regulated expression cell line is confirmed, the intracellular accumulation and secretion levels of the SOI can be evaluated by leveraging protein translation inhibitor treatments, e.g., Dox and cycloheximide.

As outlined in detail herein, regulated expression can be based on gene switches for blocking or activating mRNA synthesis by regulated coupling of transcriptional repressors or activators to constitutive or minimal promoters. In certain non-limiting embodiments, repression can be achieved by binding the repressor proteins, e.g., where the proteins sterically block transcriptional initiation, or by actively repressing transcription through transcriptional silencers. In certain non-limiting embodiments, activation of mammalian or viral enhancerless minimal promoters can be achieved by the regulated coupling to an activation domain.

In certain embodiments, the conditional coupling of transcriptional repressors or activators can be achieved by using allosteric proteins that bind the promoters in response to external stimuli. In certain embodiments, the conditional coupling of transcriptional repressors or activators can be achieved by using intracellular receptors that are released from sequestrating proteins and, thus, can bind target promoters. In certain embodiments, the conditional coupling of transcriptional repressors or activators can be achieved by using chemically induced dimerizers.

In certain embodiments, the allosteric proteins used in the regulated expression systems of the present disclosure can be proteins that modulate transcriptional activity in response to antibiotics, bacterial quorum-sensing messengers, catabolites, or to the cultivation parameters, such as temperature, e.g., cold or heat. In certain embodiments, such regulated expression systems can be catabolite-based, e.g., where a bacterial repressor that controls catabolic genes for alternative carbon sources has been transferred to mammalian cells. In certain embodiments, the repression of the target promoter can be achieved by cumate-responsive binding of the repressor CymR. In certain embodiments, the catabolite-based system can rely on the activation of chimeric promoters by 6-hydroxynicotine-responsive binding of the prokaryotic repressor HdnoR, fused to the Herpes simplex VP16 transactivation domain.

In certain embodiments, the regulated expression system of the present disclosure can employ a quorum-sensing-based expression system originated from prokaryotes that manage intra- and inter-population communication by quorum-sensing molecules. These quorum-sensing molecules bind to receptors in target cells, modulate the receptors' affinity to cognate promoters leading to the initiation of specific regulon switches. In certain embodiments, the quorum-sensing molecule can be the N-(3-oxo-octanoyl)-homoserine lactone in the presence of which, the TraR-p65 fusion protein activates expression from a minimal promoter fused to the TraR-specific operator sequence. In certain embodiments, the quorum-sensing molecule can be the butyrolactone SCB1 (racemic 2-(1′-hydroxy-6-methylheptyl)-3-(hydroxymethyl)-butanolide) in a system based on the Streptomyces coelicolor A3(2) ScbR repressor that binds its cognate operator OScbR in the absence of the SCB1. In certain embodiments, the quorum-sensing molecule can be homoserine-derived inducers used in a RTI system wherein Pseudomonas aeruginosa quorum-sensing repressors RhlR and LasR are fused to the SV40 T-antigen nuclear localization sequence and the Herpes simplex VP16 domain and can activate promoters containing specific operator sequences (las boxes).

In certain embodiments, the inducing molecules that modulate the allosteric proteins used in the regulated expression systems of the present disclosure can be, but are not limited to, cumate, isopropyl-β-D-galactopyranoside (IPTG), macrolides, 6-hydroxynicotine, doxycycline, streptogramins, NADH, tetracycline.

In certain embodiments, the intracellular receptors used in the regulated expression systems of the present disclosure can be cytoplasmic or nuclear receptors. In certain embodiments, the regulated expression systems of the present disclosure can utilize the release of transcription factors from sequestering and inhibiting proteins by using small molecules. In certain embodiments, the regulated expression systems of the present disclosure can rely on steroid-regulation, wherein a hormone receptor is fused to a natural or an artificial transcription factor that can be released from HSP90 in the cytosol, migrate into the nucleus and activate selected promoters. In certain embodiments, mutant receptors can be used that are regulated by synthetic steroid analogs in order to avoid crosstalk by endogenous steroid hormones. In certain embodiments the receptors can be an estrogen receptor variant responsive to 4-hydroxytamoxifen or a progesterone-receptor mutant inducible by RU486. In certain embodiments, the nuclear receptor-derived rosiglitazone-responsive transcription switch based on the human nuclear peroxisome proliferator-activated receptor γ(PPARγ) can be used in the regulated expression systems of the present disclosure. In certain embodiments, a variant of steroid-responsive receptors can be the RheoSwitch, that is based on a modified Choristoneura fumiferana ecdysone receptor and the mouse retinoid X receptor (RXR) fused to the Gal4 DNA binding domain and the VP16 trans-activator. In the presence of synthetic ecdysone, the RheoSwitch variant can bind and activate a minimal promoter fused to several repeats of the Gal4-response element.

In certain embodiments, the regulated expression systems disclosed herein can utilize chemically induced dimerization of a DNA-binding protein and a transcriptional activator for the activation of a minimal core promoter fused with a cognate operator. In certain embodiments, the regulated expression systems disclosed herein can utilize the rapamycin-regulated dimerization of FKBP with FRB. In this system the FRB is fused to the p65 trans-activator and FKBP is fused to a zinc finger domain specific for cognate operator sites placed upstream of an engineered minimal interleukin-12 promoter. In certain embodiments, the FKBP can be mutated. In certain embodiments, the regulated expression systems disclosed herein can utilize bacterial gyrase B subunit (GyrB), where GyrB dimerizes in the presence of the antibiotic coumermycin and dissociates with novobiocin.

In certain embodiments, the regulated expression systems of the present disclosure can be used for regulated siRNA expression. In certain embodiments, the regulated siRNA expression system can be a tetracycline, a macrolide, or an OFF- and ON-type QuoRex system. In certain embodiments, the RTI system can utilize a Xenopus terminal oligopyrimidine element (TOP), which blocks translational initiation by forming hairpin structures in the 5′ untranslated region.

In certain embodiments, the regulated expression systems described in the present disclosure can utilize gas-phase controlled expression, e.g., acetaldehyde-induced regulation (AIR) system. The AIR system can employ the Aspergillus nidulans AlcR transcription factor, which specifically activates the PAIR promoter assembled from AlcR-specific operators fused to the minimal human cytomegalovirus promoter in the presence of gaseous or liquid acetaldehyde at nontoxic concentrations.

In certain embodiments, the regulated expression systems of the present disclosure can utilize a Tet-On or a Tet-Off system. In such systems, expression of a one or more SOIs can be regulated by tetracycline or its analogue, doxycycline.

In certain embodiments, the regulated expression system of the present disclosure can utilize a PIP-on or a PIP-off system. In such systems, the expression of SOIs can be regulated by, e.g., pristinamycin, tetracycline and/or erythromycin.

### 5. Preparation and Use of RVLP-Reduced Host Cells

The presently disclosed subject matter relates to methods for the targeted integration of exogenous nucleotide sequences into a host cell. In certain embodiments, the methods relate to the integration of an exogenous nucleotide sequence into a host cell to produce a host cell suitable for subsequent targeted integration of a SOI. In certain embodiments, said methods comprise recombinase-mediated recombination. In certain embodiments, said methods involve homologous recombination, HDR, and/or NHEJ.

In certain embodiments, the presently disclosed subject matter relates to methods for the targeted integration of exogenous nucleotide sequences into a host cell in combination with transposon-mediated genomic integration of exogenous nucleotide sequences into the same host cell. In certain embodiments, the methods relate to the integration of an exogenous nucleotide sequence into a host cell to produce a host cell suitable for subsequent targeted integration of a SOI in combination with transposon-mediated genomic integration of a same or different SOI. In certain embodiments, said methods comprise recombinase-mediated recombination. In certain embodiments, said methods involve homologous recombination, HDR, and/or NHEJ.

In certain embodiments, polypeptide of interest is produced and secreted into the cell culture medium. In certain embodiments, polypeptide of interest is expressed and retained within the host cell. In certain embodiments, polypeptide of interest is expressed, inserted into, and retained in the host cell membrane.

Exogenous nucleotides of interest or vectors can be introduced into a host cell by conventional cell biology methods including, but not limited to, transfection, transduction, electroporation, or injection. In certain embodiments, exogenous nucleotides of interest or vectors are introduced into a host cell by chemical-based transfection methods comprising lipid-based transfection method, calcium phosphate-based transfection method, cationic polymer-based transfection method, or nanoparticle-based transfection. In certain embodiments, exogenous nucleotides of interest are introduced into a host cell by virus-mediated transduction including, but not limited to, lentivirus, retrovirus, adenovirus, or adeno-associated virus-mediated transduction. In certain embodiments, exogenous nucleotides of interest or vectors are introduced into a host cell via gene gun-mediated injection. In certain embodiments, both DNA and RNA molecules are introduced into a host cell using methods described herein.

### 5.1 Preparation of TI Host Cells Using Recombinase-Mediated Recombination

In certain embodiments, the present disclosure provides methods for preparing TI host cells to express a polypeptide of interest comprising: a) providing a TI host cell comprising an exogenous nucleotide sequence integrated at a site within a locus of the genome of the host cell, wherein the locus is at least about 90% homologous to SEQ ID Nos. 1-7, wherein the exogenous nucleotide sequence comprises two RRSs, flanking at least one first selection marker; b) introducing into the cell provided in a) a vector comprising two RRSs matching the two RRSs on the integrated exogenous nucleotide sequence and flanking at least one exogenous SOI and at least one second selection marker; c) introducing a recombinase, wherein the recombinase recognizes the RRSs; and d) selecting for TI cells expressing the second selection marker to thereby isolate a TI host cell expressing the polypeptide of interest.

In certain embodiments, the present disclosure provides methods for preparing TI host cells to express a polypeptide of interest comprising: a) providing a TI host cell comprising an exogenous nucleotide sequence integrated at a site within an endogenous gene selected from the group consisting of LOC107977062, LOC100768845, ITPR2, ERE67000.1, UBAP2, MTMR2, XP_003512331.2, and at least about 90% homologous sequences thereof, wherein the exogenous nucleotide sequence comprises two RRSs, flanking at least one first selection marker; b) introducing into the cell provided in a) a vector comprising two RRSs matching the two RRSs on the integrated exogenous nucleotide sequence and flanking at least one exogenous SOI and at least one second selection marker; c) introducing a recombinase, wherein the recombinase recognizes the RRSs; and d) selecting for TI cells expressing the second selection marker to thereby isolate a TI host cell expressing the polypeptide of interest.

In certain embodiments, the present disclosure provides methods for preparing TI host cells to express a polypeptide of interest comprising: a) providing a TI host cell comprising an exogenous nucleotide sequence integrated at a site within a locus of the genome of the TI host cell, wherein the locus is at least about 90% homologous to SEQ ID Nos. 1-7, wherein the exogenous nucleotide sequence comprises a first DNA cassette comprising two heterospecific RRSs, flanking at least one first selection marker; b) introducing into the cell provided in a) a vector comprising a second DNA cassette comprising two heterospecific RRSs matching the two RRSs on the integrated exogenous nucleotide sequence and flanking at least one exogenous SOI and at least one second selection marker; c) introducing a recombinase, wherein the recombinase recognizes the RRSs and performs one RMCE; and d) selecting for TI cells expressing the second selection marker to thereby isolate a TI host cell expressing the polypeptide of interest.

In certain embodiments, the present disclosure provides methods for preparing TI host cells to express a polypeptide of interest comprising: a) providing a TI host cell comprising an exogenous nucleotide sequence integrated at a site within an endogenous sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, XP_003512331.2, and at least about 90% homologous sequences thereof, wherein the exogenous nucleotide sequence comprises a first DNA cassette comprising two heterospecific RRSs, flanking at least one first selection marker; b) introducing into the cell provided in a) a vector comprising a second DNA cassette comprising two heterospecific RRSs matching the two RRSs on the integrated exogenous nucleotide sequence and flanking at least one exogenous SOI and at least one second selection marker; c) introducing a recombinase, wherein the recombinase recognizes the RRSs and performs one RMCE; and d) selecting for TI cells expressing the second selection marker to thereby isolate a TI host cell expressing the polypeptide of interest.

In certain embodiments, the present disclosure provides methods for preparing TI host cells to express a first and second polypeptide of interest (where the first and second polypeptides can be the same or different) comprising: a) providing a TI host cell comprising an exogenous nucleotide sequence integrated at a site within a locus of the genome of the host cell, wherein the locus comprises a sequence that is at least about 90% homologous to all or a portion of the contig sequence of one of the contigs NW_006874047.1, NW_006884592.1, NW_006881296.1, NW 003616412.1, NW 003615063.1, NW_006882936.1, and NW_003615411.1 or to a SEQ ID Nos. 1-7, wherein the exogenous nucleotide sequence comprises a first and a second RRS flanking at least one first selection marker, and a third RRS located between the first and the second RRS, and all the RRSs are heterospecific; b) introducing into the cell provided in a) a first vector comprising two RRSs matching the first and the third RRS on the integrated exogenous nucleotide sequence and flanking at least one first exogenous SOI and at least one second selection marker; c) introducing into the cell provided in a) a second vector comprising two RRSs matching the second and the third RRS on the integrated exogenous nucleotide sequence and flanking at least one second exogenous SOI; d) introducing one or more recombinases, wherein the one or more recombinases recognize the RRSs; and e) selecting for TI cells expressing the second selection marker to thereby isolate a TI host cell expressing the first and second polypeptides of interest. In certain embodiments, rather than have the entire selection maker on the first vector, the first vector comprises a promoter sequence operably linked to the codon ATG positioned flanked upstream by the first SOI and downstream by an RRS; and the second vector comprises a selection marker lacking an ATG transcription start codon flanked upstream by an RRS and downstream by the second SOI.

In certain embodiments, the present disclosure provides methods for preparing TI host cells to express a first and second polypeptide of interest (where the first and second polypeptides can be the same or different) comprising: a) providing a TI host cell comprising an exogenous nucleotide sequence integrated at a site within an endogenous sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, XP_003512331.2, and at least about 90% homologous sequences thereof, wherein the exogenous nucleotide sequence comprises a first and a second RRS flanking at least one first selection marker, and a third RRS located between the first and the second RRS, and all the RRSs are heterospecific; b) introducing into the cell provided in a) a first vector comprising two RRSs matching the first and the third RRS on the integrated exogenous nucleotide sequence and flanking at least one first exogenous SOI and at least one second selection marker; c) introducing into the cell provided in a) a second vector comprising two RRSs matching the second and the third RRS on the integrated exogenous nucleotide sequence and flanking at least one second exogenous SOI; d) introducing one or more recombinases, wherein the one or more recombinases recognize the RRSs; and e) selecting for TI cells expressing the second selection marker to thereby isolate a TI host cell expressing the first and second polypeptides of interest. In certain embodiments, rather than have the entire selection maker on the first vector, the first vector comprises a promoter sequence operably linked to the codon ATG positioned flanked upstream by the first SOI and downstream by an RRS; and the second vector comprises a selection marker lacking an ATG transcription start codon flanked upstream by an RRS and downstream by the second SOI.

In certain embodiments, the present disclosure provides methods for preparing TI host cells to express a first and second polypeptide of interest (where the first and second polypeptides can be the same or different) comprising: a) providing a TI host cell comprising an exogenous nucleotide sequence integrated at a site within a locus of the genome of the host cell, wherein the locus is at least about 90% homologous to a sequence comprising all or a portion of the contig sequence of one of the contigs NW_006874047.1, NW_006884592.1, NW_006881296.1, NW 003616412.1, NW_003615063.1, NW_006882936.1, and NW_003615411.1 or to SEQ ID Nos. 1-7, wherein the exogenous nucleotide sequence comprises a first DNA cassette comprising a first and a second RRS flanking at least one first selection marker, and a third RRS located between the first and the second RRS, and all three RRSs are heterospecific; b) introducing into the cell provided in a) a first vector comprising a second DNA cassette, wherein the second DNA cassette comprises two heterospecific RRSs matching the first and the third RRS of the first DNA cassette and flanking at least one first exogenous SOI and at least one second selection marker; c) introducing into the cell provided in a) a second vector comprising a third DNA cassette, wherein the third DNA cassette comprises two heterospecific RRSs matching the second and the third RRS of the first DNA cassette and flanking at least one second exogenous SOI; d) introducing one or more recombinases, wherein the one or more recombinases recognize the RRSs and perform two RMCEs; and e) selecting for TI cells expressing the second selection marker to thereby isolate a TI host cell expressing the first and second polypeptides of interest. In certain embodiments, rather than have the entire selection maker on the first vector, the first vector comprises a promoter sequence operably linked to the codon ATG positioned flanked upstream by the first SOI and downstream by an RRS; and the second vector comprises a selection marker lacking an ATG transcription start codon flanked upstream by an RRS and downstream by the second SOI.

In certain embodiments, the present disclosure provides methods for preparing TI host cells to express a first and second polypeptide of interest (where the first and second polypeptides can be the same or different) comprising: a) providing a TI host cell comprising an exogenous nucleotide sequence integrated at a site within an endogenous sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, XP_003512331.2, and sequences at least about 90% homologous thereto, wherein the exogenous nucleotide sequence comprises a first DNA cassette comprising a first and a second RRS flanking at least one first selection marker, and a third RRS located between the first and the second RRS, and all three RRSs are heterospecific; b) introducing into the cell provided in a) a first vector comprising a second DNA cassette, wherein the second DNA cassette comprises two heterospecific RRSs matching the first and the third RRS of the first DNA cassette and flanking at least one first exogenous SOI and at least one second selection marker; c) introducing into the cell provided in a) a second vector comprising a third DNA cassette, wherein the third DNA cassette comprises two heterospecific RRSs matching the second and the third RRS of the first DNA cassette and flanking at least one second exogenous SOI; d) introducing one or more recombinases, wherein the one or more recombinases recognize the RRSs and perform two RMCEs; and e) selecting for TI cells expressing the second selection marker to thereby isolate a TI host cell expressing the first and second polypeptides of interest. In certain embodiments, rather than have the entire selection maker on the first vector, the first vector comprises a promoter sequence operably linked to the codon ATG positioned flanked upstream by the first SOI and downstream by an RRS; and the second vector comprises a selection marker lacking an ATG transcription start codon flanked upstream by an RRS and downstream by the second SOI.

In certain embodiments, the present disclosure provides methods for preparing TI host cells to express a polypeptide of interest comprising: a) providing a TI host cell comprising an exogenous nucleotide sequence integrated at a site within a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to SEQ ID Nos. 1-7, wherein the exogenous nucleotide sequence comprises one RRS adjacent to at least one first selection marker; b) introducing into the cell provided in a) a vector comprising one RRS matching the RRS on the integrated exogenous nucleotide sequence and adjacent to at least one exogenous SOI and at least one second selection marker; c) introducing a recombinase, wherein the recombinase recognizes the RRSs; and d) selecting for TI cells expressing the second selection marker to thereby isolate a TI host cell expressing the polypeptide of interest.

In certain embodiments, the present disclosure provides methods for preparing TI host cells to express a polypeptide of interest comprising: a) providing a TI host cell comprising an exogenous nucleotide sequence integrated at a site within an endogenous sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, XP_003512331.2, and sequences at least about 90% homologous thereto, wherein the exogenous nucleotide sequence comprises one RRS adjacent to at least one first selection marker; b) introducing into the cell provided in a) a vector comprising one RRS matching the RRS on the integrated exogenous nucleotide sequence and adjacent to at least one exogenous SOI and at least one second selection marker; c) introducing a recombinase, wherein the recombinase recognizes the RRSs; and d) selecting for TI cells expressing the second selection marker to thereby isolate a TI host cell expressing the polypeptide of interest.

The presently disclosed subject matter also relates to methods of producing a polypeptide of interest comprising: a) providing a TI host cell described herein; b) culturing the TI host cell in a) under conditions suitable for expressing the SOI and recovering a polypeptide of interest therefrom.

In certain embodiments, the present disclosure provides methods for preparing TI host cells suitable for subsequent targeted integration comprising: a) providing a TI host cell comprising an exogenous nucleotide sequence integrated at a site within a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7, wherein the exogenous nucleotide sequence comprises two RRSs flanking at least one exogenous SOI and at least one first selection marker; b) introducing into the cell provided in a) a vector comprising two RRSs matching the two RRSs on the integrated exogenous nucleotide sequence and flanking at least one second selection marker; c) introducing a recombinase, wherein the recombinase recognizes the RRSs; and d) selecting for TI cells expressing the second selection marker to thereby isolate a TI host cell suitable for subsequent targeted integration.

In certain embodiments, the present disclosure provides methods for preparing TI host cells suitable for subsequent targeted integration comprising: a) providing a TI host cell comprising an exogenous nucleotide sequence integrated at a site within an endogenous sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, XP_003512331.2, and sequences at least about 90% homologous thereto, wherein the exogenous nucleotide sequence comprises two RRSs flanking at least one exogenous SOI and at least one first selection marker; b) introducing into the cell provided in a) a vector comprising two RRSs matching the two RRSs on the integrated exogenous nucleotide sequence and flanking at least one second selection marker; c) introducing a recombinase, wherein the recombinase recognizes the RRSs; and d) selecting for TI cells expressing the second selection marker to thereby isolate a TI host cell suitable for subsequent targeted integration.

In certain embodiments, the present disclosure provides methods for preparing TI host cells suitable for subsequent targeted integration comprising: a) providing a TI host cell comprising an exogenous nucleotide sequence integrated at a site within a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7, wherein the exogenous nucleotide sequence comprises a first and a second RRS flanking at least one exogenous SOI and at least one first selection marker; b) introducing into the cell provided in a) a vector comprising three RRSs, wherein the first RRS of the vector matches the first RRS on the integrated exogenous nucleotide sequence, the second RRS of the vector matches the second RRS on the integrated exogenous nucleotide sequence, and at least one second selection marker located between the first and the second RRS; c) introducing a recombinase, wherein the recombinase recognizes the first and the second RRS on both the vector and the integrated exogenous nucleotide sequence; and d) selecting for TI host cells expressing the second selection marker to thereby isolate a TI host cell suitable for subsequence targeted integration.

In certain embodiments, the present disclosure provides methods for preparing TI host cells suitable for subsequent targeted integration comprising: a) providing a TI host cell comprising an exogenous nucleotide sequence integrated at a site within an endogenous sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a gene selected from the group consisting of LOC107977062, LOC100768845, ITPR2, ERE67000.1, UBAP2, MTMR2, XP_003512331.2, and sequences at least about 90% homologous thereto, wherein the exogenous nucleotide sequence comprises a first and a second RRS flanking at least one exogenous SOI and at least one first selection marker; b) introducing into the cell provided in a) a vector comprising three RRSs, wherein the first RRS of the vector matches the first RRS on the integrated exogenous nucleotide sequence, the second RRS of the vector matches the second RRS on the integrated exogenous nucleotide sequence, and at least one second selection marker located between the first and the second RRS; c) introducing a recombinase, wherein the recombinase recognizes the first and the second RRS on both the vector and the integrated exogenous nucleotide sequence; and d) selecting for TI host cells expressing the second selection marker to thereby isolate a TI host cell suitable for subsequent targeted integration.

### 5.2 Methods for Targeted Modification of a Host Cell Using Homologous Recombination, HDR, or NHEJ

In certain embodiments, the present disclosure provides methods for preparing TI host cells to express a polypeptide of interest comprising: a) providing a TI host cell comprising a locus of the genome of the host cell, wherein the locus is at least about 90% homologous to SEQ ID Nos. 1-7; b) introducing a vector into the TI host cell, wherein the vector comprises nucleotide sequences at least 50% homologous to a sequence selected from SEQ ID No. 1-7 flanking a DNA cassette, wherein the DNA cassette comprises at least one selection marker and at least one exogenous SOI; c) selecting for the selection marker to isolate a TI host cell with the SOI integrated in the locus of the genome, and expressing the polypeptide of interest. In certain embodiments, the DNA cassette of the vector further comprises at least one selection marker and at least one exogenous SOI flanked by two RRSs.

In certain embodiments, the present disclosure provides methods for preparing TI host cells to express a polypeptide of interest comprising g: a) providing a TI host cell comprising a locus of the genome of the host cell, wherein the locus is at least about 90% homologous to a sequence selected from SEQ ID Nos. 1-7; b) introducing a polynucleotide into the host cell, wherein the polynucleotide comprises nucleotide sequences at least 50% homologous to a sequence selected from SEQ ID No. 1-7 flanking a DNA cassette, wherein the DNA cassette comprises at least one selection marker and at least one exogenous SOI; c) selecting for the selection marker to isolate a TI host cell with the SOI integrated in the locus of the genome, and expressing the polypeptide of interest. In certain embodiments, the DNA cassette of the vector further comprises at least one selection marker and at least one exogenous SOI flanked by two RRSs.

In certain embodiments, the homologous recombination is facilitated by an integrating vector. In certain embodiments, a vector is selected from the group consisting of an adenovirus vector, an adeno-associated virus vector, a lentivirus vector, a retrovirus vector, an integrating phage vector, a non-viral vector, a transposon and/or transposase vector, an integrase substrate, and a plasmid. In certain embodiments, the transposon can be a piggyBac (PB) transposon system.

In certain embodiments, the integration is promoted by an exogenous nuclease. In certain embodiments, the exogenous nuclease is selected from the group consisting of a zinc finger nuclease (ZFN), a ZFN dimer, a transcription activator-like effector nuclease (TALEN), a TAL effector domain fusion protein, an RNA-guided DNA endonuclease, an engineered meganuclease, and a clustered regularly interspaced short palindromic repeats (CRISPR)-associated (Cas) endonuclease.

In certain embodiments, the present disclosure provides methods for preparing TI host cells suitable for subsequent targeted integration comprising: a) providing a TI host cell comprising a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7; b) introducing a vector into the TI host cell, wherein the vector comprises nucleotide sequences at least 50% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequences selected from SEQ ID Nos. 1-7 flanking a DNA cassette, wherein the DNA cassette comprises at least one selection marker flanked by two RRSs; c) selecting for the selection marker to isolate a TI host cell suitable for subsequent targeted integration.

In certain embodiments, the present disclosure provides methods for preparing TI host cells suitable for subsequent targeted integration comprising: a) providing a TI host cell comprising a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7; b) introducing a polynucleotide into the TI host cell, wherein the polynucleotide comprises nucleotide sequences at least 50% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7 flanking a DNA cassette, wherein the DNA cassette comprises at least one selection marker flanked by two RRSs; c) selecting for the selection marker to isolate a TI host cell suitable for subsequent targeted integration.

In certain embodiments, the present disclosure provides methods for preparing TI host cells suitable for subsequent targeted integration comprising: a) providing a TI host cell comprising a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7; b) introducing a vector into the host cell, wherein the vector comprises nucleotide sequences at least 50% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7 flanking a DNA cassette, wherein the DNA cassette comprises three RRSs, wherein the third RRS and at least one selection marker is located between the first and the second RRS; and c) selecting for the selection marker to isolate a TI host cell suitable for subsequent targeted integration.

In certain embodiments, the present disclosure provides methods for preparing TI host cells suitable for subsequent targeted integration comprising: a) providing a TI host cell comprising a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7; b) introducing a polynucleotide into the host cell, wherein the polynucleotide comprises nucleotide sequences at least 50% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7 flanking a DNA cassette, wherein the DNA cassette comprises three RRSs, wherein the third RRS and at least one selection marker is located between the first and the second RRS; and c) selecting for the selection marker to isolate a TI host cell suitable for subsequent targeted integration.

In certain embodiments, the present disclosure provides methods for preparing a TI host cell expressing at least one polypeptide of interest comprising: a) providing a TI host cell comprising at least one exogenous nucleotide sequence integrated at a site within one or more loci of the genome of the TI host cell, wherein the one or more loci are at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7, wherein the at least one exogenous nucleotide sequence comprises two RRSs, flanking at least one first selection marker; b) introducing into the cell provided in a) a vector comprising two RRSs matching the two RRSs on the integrated exogenous nucleotide sequence and flanking at least one exogenous SOI and at least one second selection marker; c) introducing a recombinase or a nucleic acid encoding a recombinase, wherein the recombinase recognizes the RRSs; and selecting for TI cells expressing the second selection marker to thereby isolate a TI host cell expressing the at least one polypeptide of interest.

In certain embodiments, the present disclosure provides methods for preparing a TI host cell expressing at least one first and second polypeptide of interest (where the first and second polypeptides can be the same or different) comprising: a) providing a TI host cell comprising at least one exogenous nucleotide sequence integrated at a site within one or more loci of the genome of the host cell, wherein one or more loci are at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7, wherein the exogenous nucleotide sequence comprises a first and a second RRS flanking at least one first selection marker, and a third RRS located between the first and the second RRS, and all the RRSs are heterospecific; b) introducing into the cell provided in a) a first vector comprising two RRSs matching the first and the third RRS on the at least one integrated exogenous nucleotide sequence and flanking at least one first exogenous SOI and at least one second selection marker; c) introducing into the cell provided in a) a second vector comprising two RRSs matching the second and the third RRS on the at least one integrated exogenous nucleotide sequence and flanking at least one second exogenous SOI; d) introducing one or more recombinases, or one or more nucleic acids encoding one or more recombinases, wherein the one or more recombinases recognize the RRSs; and e) selecting for TI cells expressing the second selection marker to thereby isolate a TI host cell expressing the at least one first and second polypeptides of interest. In certain embodiments, rather than have the entire selection maker on the first vector, the first vector comprises a promoter sequence operably linked to the codon ATG positioned flanked upstream by the first SOI and downstream by an RRS; and the second vector comprises a selection marker lacking an ATG transcription start codon flanked upstream by an RRS and downstream by the second SOI.

In certain embodiments, the present disclosure provides methods for preparing a TI host cell expressing a polypeptide of interest comprising: a) providing a TI host cell comprising at least one exogenous nucleotide sequence integrated at a site within one or more loci of the genome of the TI host cell, wherein the one or more loci are at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7, wherein the exogenous nucleotide sequence comprises one or more RRSs; b) introducing into the cell provided in a) a vector comprising one or more RRSs matching the one or more RRSs on the integrated exogenous nucleotide sequence and flanking at least one exogenous SOI operably linked to a regulatable promoter; c) introducing a recombinase or a nucleic acid encoding a recombinase, wherein the recombinase recognizes the RRSs; and d) selecting for TI cells expressing the exogenous SOI in the presence of an inducer to thereby isolate a TI host cell expressing the polypeptide of interest.

In certain embodiments, the present disclosure provides methods for expressing a polypeptide of interest comprising: a) providing a host cell comprising at least one exogenous SOI flanked by two RRSs and a regulatable promoter integrated within a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7; and b) culturing the cell under conditions suitable for expressing the SOI and recovering a polypeptide of interest therefrom.

In certain embodiments, the present disclosure provides methods for preparing a TI host cell expressing a first and second polypeptide of interest (where the first and second polypeptides can be the same or different) comprising: a) providing a TI host cell comprising an exogenous nucleotide sequence integrated at a site within a locus of the genome of the host cell, wherein the locus is at least about 90% homologous to a sequence comprising all or a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7, wherein the exogenous nucleotide sequence comprises a first, second RRS and a third RRS located between the first and the second RRS, and all the RRSs are heterospecific; b) introducing into the cell provided in a) a first vector comprising two RRSs matching the first and the third RRS on the integrated exogenous nucleotide sequence and flanking at least one first exogenous SOI operably linked to a regulatable promoter; c) introducing into the cell provided in a) a second vector comprising two RRSs matching the second and the third RRS on the integrated exogenous nucleotide sequence and flanking at least one second SOI operably linked to a regulatable promoter; d) introducing one or more recombinases, or one or more nucleic acids encoding one or more recombinases, wherein the one or more recombinases recognize the RRSs; and e) selecting for TI cells expressing the at least first and second exogenous SOIs in the presence of an inducer to thereby isolate a TI host cell expressing the polypeptides of interest. In certain embodiments, rather than have the entire selection maker on the first vector, the first vector comprises a promoter sequence operably linked to the codon ATG positioned flanked upstream by the first SOI and downstream by an RRS; and the second vector comprises a selection marker lacking an ATG transcription start codon flanked upstream by an RRS and downstream by the second SOI.

### 6. Products

The RVLP-reduced CHO host cells of the present disclosure can be used for the expression of any molecule of interest, e.g., a polypeptide of interest. In certain embodiments, the host cells of the present disclosure can be used for the expression of polypeptides, e.g., mammalian polypeptides. Non-limiting examples of such polypeptides include hormones, receptors, fusion proteins, regulatory factors, growth factors, complement system factors, enzymes, clotting factors, anti-clotting factors, kinases, cytokines, CD proteins, interleukins, therapeutic proteins, diagnostic proteins and antibodies. In some embodiments, the antibody is a monoclonal antibody. In some embodiments, the antibody is a therapeutic antibody. In some embodiments, the antibody is a diagnostic antibody. In some embodiments, the antibody is a human antibody. In some embodiments, the antibody is a humanized antibody.

In certain embodiments, the polypeptide of interest is a bi-specific, tri-specific or multispecific polypeptide, e.g., a bi-specific antibody. Various molecular formats for multispecific antibodies are known in the art and are included herein (see e.g., Spiess et al., Mol Immunol 67 (2015) 95-106). A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Other examples of bispecific antibody formats include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

Therapeutic antibodies include, without limitation, anti-HER receptor family antibodies (such as anti-HER1 (EGFR), anti-HER2, anti-HER3 and anti-HER4); anti-CD protein antibodies (such as anti-CD3, anti-CD4, anti-CD8, anti-CD19, anti-CD20, anti-CD21, anti-CD22, anti-CD25, anti-CD33, anti-CD34, anti-CD38, anti-CD52); anti-IL-8 antibodies; anti-VEGF antibodies; anti-CD40 antibodies, anti-CD11a antibodies; anti-CD18 antibodies; anti-IgE antibodies; anti-Apo-2 receptor antibodies; anti-Tissue Factor (TF) antibodies; anti-cell adhesion molecules such as LFA-1, Mol, p150,95, VLA-4, ICAM-1, VCAM, anti-human α₄β₇ integrin antibodies, anti-human αᵥβ₈ integrin antibodies, anti-αvβ3 antibodies including either α or ρ or subunits thereof (e.g. anti-CD11a, anti-CD18 or anti-CD11b antibodies); anti-EGFR antibodies; anti-Fc receptor antibodies; anti-carcinoembryonic antigen (CEA) antibodies; anti-human renal cell carcinoma antibodies; anti-human colorectal tumor antibodies; anti-human melanoma antibody R24 directed against GD3 ganglioside; anti-human squamous-cell carcinoma; antibodies directed against breast epithelial cells; antibodies that bind to colon carcinoma cells; anti-EpCAM antibodies; anti-GpIIb/IIIa antibodies; anti-RSV antibodies; anti-CMV antibodies; anti-HIV antibodies; anti-hepatitis antibodies; anti-CA 125 antibodies; anti-human 17-1A antibodies; and anti-human leukocyte antigen (HLA) antibodies, and anti-HLA DR antibodies; anti-growth factors such as vascular endothelial growth factor (anti-VEGF) or fragments; anti-IgE; anti-blood group antigens; anti-flk2/flt3 receptor; and anti-obesity (OB) receptor. Other exemplary proteins to which therapeutic antibodies are designed include anti-amyloid antibodies, anti-alpha-synuclein (e.g.: prasinezumab), anti-amyloid-beta, anti-growth hormone (GH), including human growth hormone (hGH) and bovine growth hormone (bGH); growth hormone releasing factor; parathyroid hormone; thyroid stimulating hormone; lipoproteins; α-1-antitrypsin; insulin A-chain; insulin B-chain; proinsulin; follicle stimulating hormone; calcitonin; luteinizing hormone; glucagon; clotting factors such as factor VIIIC, tissue factor or von Willebrands factor; anti-clotting factors such as Protein C; atrial natriuretic factor; lung surfactant; a plasminogen activator, such as urokinase or tissue-type plasminogen activator (t-PA); bombazine; thrombin; tumor necrosis factor-α and -β; enkephalinase; RANTES (regulated on activation normally T-cell expressed and secreted); human macrophage inflammatory protein (MIP-1-α); serum albumin such as human serum albumin (HSA); mullerian-inhibiting substance; relaxin A-chain; relaxin B-chain; prorelaxin; mouse gonadotropin-associated peptide; DNase; inhibin; activin; receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins (IGFBPs); erythropoietin (EPO); thrombopoietin (TPO); osteoinductive factors; immunotoxins; a bone morphogenetic protein (BMP); an interferon such as interferon−α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor (DAF); a viral antigen such as, for example, a portion of the AIDS envelope; transport proteins; homing receptors; addressins; regulatory proteins; immunoadhesins; and biologically active fragments or variants of any of the above-listed polypeptides. Many other antibodies and/or other proteins may be used in accordance with the instant invention, and the above lists are not meant to be limiting.

Therapeutic antibodies of particular interest include those in clinical practice or in development, such as commercially available AVASTIN® (bevacizumab), HERCEPTIN® (trastuzumab), LUCENTIS® (ranibizumab), RAPTIVA® (efalizumab), RITUXAN® (rituximab), and XOLAIR® (omalizumab), anti-amyloid beta (Abeta), anti-CD4 (MTRX1011A), anti-EGFL7 (EGF-like-domain 7), anti-IL13, Apomab (anti-DR5-targeted pro-apoptotic receptor agonist (PARA), anti-BR3 (CD268, anti-BLyS receptor 3, anti-BAFF-R, (BAFF Receptor), anti-beta 7 integrin subunit, anti-αᵥβ₈ integrin antibodies, dacetuzumab (Anti-CD40), GA101 (anti-CD20 monoclonal antibody), MetMAb (anti-MET receptor tyrosine kinase), anti-neuropilin-1 (NRP1), OCREVUS® (ocrelizumab—anti-CD20 antibody), anti-OX40 ligand, anti-oxidized LDL (oxLDL), PERJETA® (pertuzumab—HER dimerization inhibitors (HDIs)), TECENTRIQ® (anti-PD-L1 antibody), anti-CD79b antibody, LUNSUMIO® or COLUMVI™ (anti-CD20×anti-CD3 bispecific antibody), VABYSMO® (anti-VEGF-A×anti-angiopoietin-2 bispecific antibody), rhuMAb IFN alpha, etc. Many other antibodies and/or other proteins may be used in accordance with the instant invention, and the above lists are not meant to be limiting.

The host cells of the present disclosure may be employed in the production of a molecule of interest at manufacturing scale. “Manufacturing scale” production of therapeutic proteins, or other proteins, utilize cell cultures ranging from about 400 L to about 80,000 L, depending on the protein being produced and the need. Typically, such manufacturing scale production utilizes cell culture sizes from about 400 L to about 25,000 L. Within this range, specific cell culture sizes such as 4,000 L, about 6,000 L, about 8,000, about 10,000, about 12,000 L, about 14,000 L, or about 16,000 L may be utilized.

The host cells of the present disclosure can be employed in the production of large quantities of a molecule of interest in a shorter timeframe as compared to non-TI cells used in current cell culture methods. In certain embodiments, the host cells of the present disclosure can be employed for improved quality of the molecule of interest as compared to non-TI cells used in current cell culture methods. In certain embodiments, the host cells of the present disclosure can be used to enhance seed train stability by preventing chronic toxicity that can be caused by products that can cause cell stress and clonal instability over time. In certain embodiments, the host cells of the present disclosure can be used for the optimal expression of acutely toxic products.

In certain embodiments, the host cells, the TI systems of the present disclosure, can be used for cell culture process optimization and/or process development.

In certain embodiments, the host cells of the present disclosure can be used to accelerate the production of a molecule of interest by about 1 week, about, 2 weeks, about 3 weeks, about 4 weeks, about 5 weeks, about 6 weeks, about 7 weeks, about 8 weeks, about 9 weeks, or about 10 weeks as compared to non-TI cells used in conventional cell culture methods. In certain embodiments, the host cells of the present disclosure can be used to accelerate the harvest of a molecule of interest by about 1 week, about, 2 weeks, about 3 weeks, about 4 weeks, about 5 weeks, about 6 weeks, about 7 weeks, about 8 weeks, about 9 weeks, or about 10 weeks as compared to non-TI cells used in conventional cell culture methods.

In certain embodiments, the host cells of the present embodiment can be employed to reduce aggregate levels of a molecule of interest as compared to non-TI cells used in conventional cell culture methods.

In certain embodiments, the host cells of the present disclosure can be used to achieve increased expression of a polypeptide (or polypeptides) of interest relative to a host cell where the exogenous sequence expressing the polypeptide (or polypeptides) of interest is randomly integrated. For example, but not by way of limitation, the host cells of the present disclosure can achieve expression of standard and half antibodies at titers of at least 3 g/L, 3.5 g/L, 4 g/L, 4.5 g/L, 5 g/L, 5.5 g/L, 6 g/L, 6.5 g/L, 7 g/L, 7.5 g/L, 8 g/L, 8.5 g/L, 9 g/L, 9.5 g/L, 10 g/L, 10.5 g/L, 11 g/L, or more, and expression of multispecific antibodies, e.g., bispecific antibodies, of at least 1.5 g/L, 2 g/L, 2.5 g/L, 3 g/L, 3.5 g/L, 4 g/L, 4.5 g/L, 5 g/L, 5.5 g/L, 6 g/L, or more. In certain embodiments, the host cells of the present disclosure can achieve increased bispecific content relative to a host cell where the exogenous sequence(s) expressing the bispecific content is randomly integrated. For example, but not by way of limitation the host cells of the present disclosure can achieve bispecific content of at least 80%, 85%, 90%, 95%, 96%, 98%, 99% or more.

In certain embodiments, the host cells of the present disclosure can be used as an investigational tool. In certain embodiments, the host cells of the present disclosure can be used as a diagnostic tool to map out the root causes of low protein expression for problematic molecules in various cells. In certain embodiments, the host cells of the present disclosure can be used to directly link an observed phenomenon or cellular behavior to the transgene expression in the cells. The host cell of the present disclosure can also be used to demonstrate whether or not an observed behavior is reversible in the cells. In certain embodiments, the host cells of the present disclosure can be exploited to identify and mitigate problems with respect to transgene(s) transcription and expression in cells.

### 7. Examples

### 7.1 Inactivation of ERV Genes: RVLP Titer & Integral Viable Cell Concentration

CRISPR Reagents. Guide RNAs targeting the Gag genes of CHERV-3g and CHERV-1b for indel (ID) knockout were designed using CRISPOR software (Concordet J P, Haeussler M. CRISPOR: intuitive guide selection for CRISPR/Cas9 genome editing experiments and screens. Nucleic Acids Res. 2018 Jul. 2; 46(W1):W242-W245. doi: 10.1093/nar/gky354). Guide RNAs targeting the Gag genes of CHERV-3g and CHERV-1b for base edit (BE) knockout were designed using BE-Designer software (Hwang, G H., Park, J., Lim, K. et al. Web-based design and analysis tools for CRISPR base editing. BMC Bioinformatics 19, 542 (2018). https://doi.org/10.1186/s12859-018-2585-4). All ID guide RNAs were chemically synthesized by Integrated DNA Technologies, Inc.

Guide RNAs targeting the genomic flanking regions of CHERV-3g and CHERV-1b for deletion (DL) knockout were designed using CRISPOR software (Concordet J P, Haeussler M. CRISPOR: intuitive guide selection for CRISPR/Cas9 genome editing experiments and screens. Nucleic Acids Res. 2018 Jul. 2; 46(W1):W242-W245. doi: 10.1093/nar/gky354). All DL guide RNAs were chemically synthesized by Integrated DNA Technologies, Inc.

Cas9 nuclease was purchased from Integrated DNA Technologies, Inc., and a plasmid for the expression of AncBE4max (Koblan, L., Doman, J., Wilson, C. et al. Improving cytidine and adenine base editors by expression optimization and ancestral reconstruction. Nat Biotechnol 36, 843-846 (2018). https://doi.org/10.1038/nbt.4172) was chemically synthesized by Genscript Biotech.

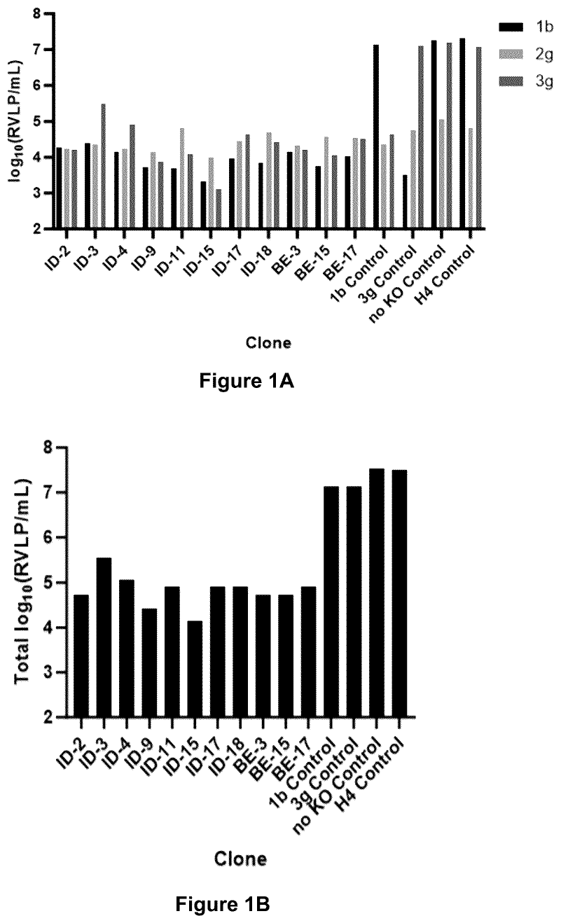

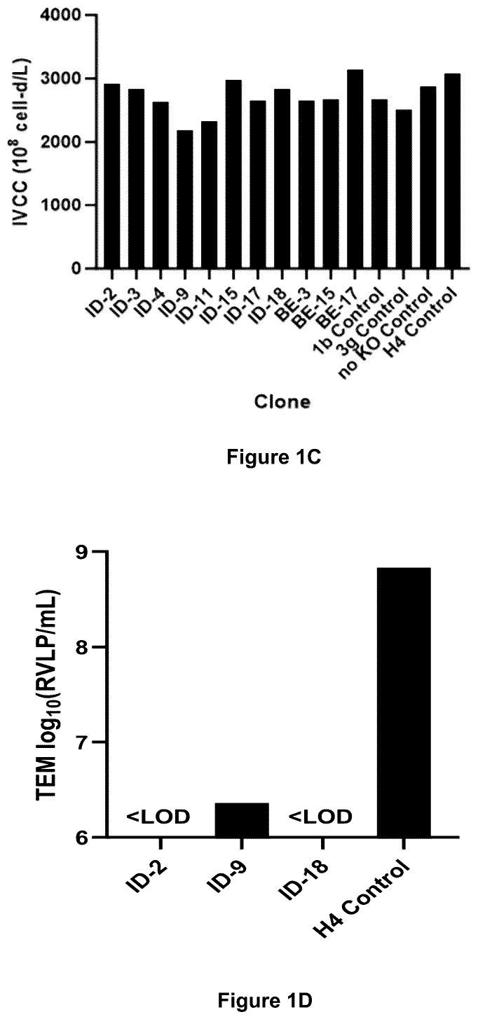

Transfection. For the results depicted in FIG. 1A-1D, a CHO-K1-derived targeted integration host cell line, Line A, with a known deletion in the ETC109F locus was used to conduct knockouts. For indel knockouts, Cas9 protein and CHERV-3g Gag sgRNA and CHERV-1b Gag sgRNA were complexed into RNPs and electroporated into cells with a Neon Transfection system (ThermoFisher). After four days, cells were transfected again under the same conditions. For base-edit knockouts, AncBE4max plasmid and CHERV-3g Gag sgRNA and CHERV-1b Gag sgRNA were electroporated into cells with a Neon Transfection system (ThermoFisher).

With respect to the results depicted in FIG. 2B-2C, A CHO-K1-derived targeted integration host cell line, “host control”, with a known deletion in the ETC109F locus was used to conduct knockouts. For deletion knockouts, Cas9 protein and CHERV-3g 5′ Flanking sgRNA, CHERV-3g 3′ Flanking sgRNA, CHERV-1b 5′ Flanking sgRNA and CHERV-1b 3′ Flanking sgRNA were complexed into RNPs and electroporated into cells with a Neon Transfection system (ThermoFisher). After two days, cells were transfected again under the same conditions.

Single Cell Cloning. For the results depicted in FIG. 1A-1D, knockout pools were subjected to single cell cloning into 384-well plates by limiting dilution (0.4 cells/well). Confluent wells on day 14 were scaled up to 96-well plates. Supernatant of cultures were treated with Turbo DNase (ThermoFisher) and CHERV-3g, CHERV-1b, and Actb titers were quantified by One-Step RT-ddPCR Advanced Kit for Probes (Bio-Rad). Clones with lowest CHERV-3g+CHERV-1b titers (relative to Actb) were expanded, along with control clones without CHERV-3g KO, CHERV-1b KO, or both CHERV-3g and CHERV-1b KO.

For the results depicted in FIG. 2B-2C, knockout pools were subjected to single cell cloning into 384-well plates using an UP.SIGHT single cell dispenser (Cytena). Confluent wells on day 14 were scaled up to 96-well plates. Clones were screened for large deletions in CHERV-3g and CHERV-1b loci by genomic PCR. Additionally, supernatant of cultures were treated with Turbo DNase (ThermoFisher) and CHERV-3g, CHERV-1b, and Actb titers were quantified by One-Step RT-ddPCR Advanced Kit for Probes (Bio-Rad). Clones with genotypes of interest were expanded and assessed by production culture.

Production Culture. RVLP KO clones were cultured in a 7-day fed-batch culture in shake flasks using chemically-defined medium and feeds. Cell counts were periodically taken to calculate integral viable cell concentration (IVCC). Supernatant was harvested on day 7 and treated with Turbo DNase (ThermoFisher). RNA was extracted using the MagNA Pure 96 system (Roche Diagnostics). Extracted RNA was assessed for CHERV-3g, CHERV-1b, and CHERV-2g titers by One-Step RT-ddPCR Advanced Kit for Probes (Bio-Rad). Total RVLP titers were calculated by summing CHERV-3g, CHERV-1b, and CHERV-2g titers.

TEM Titration. Cell cultures were harvested by centrifugation at 250×g for 10 minutes. Supernatant was submitted to Charles River Laboratories for RVLP quantification by TEM analysis. In brief, 40 mL of supernatant was ultracentrifuged and pellets were fixed in 2% glutaraldehyde and 0.1 M sodium cacodylate. Thin sections of the pellet were cut at 70-90 nm and mounted on 200 mesh copper grids, stained with methanolic uranyl acetate and Reynold's lead citrate, and examined by TEM. Trained technicians identified and counted ten grid spaces for particles with retrovirus-like morphology. RVLP titers were calculated based on grid section volume, pellet volume, and sample volume.

Results. FIG. 1A depicts the distribution of expression of three ERV species in RVLP Gag knockout clones and controls where ID correspond to indel knockouts and BE correspond to Base Edits as discussed above. FIG. 1B depicts the total RVLP titers (sum of three ERVs) in Gag knockout clones and controls. FIG. 1C depicts the production culture integral viable cell concentration (IVCC) in Gag knockout clones and controls. FIG. 1D indicates that Gag knockout clones produced fewer RVLPs, as assessed by TEM. In fact, in two of three knockout clones, RVLP titers were below the limit of detection of the assay.

FIG. 2A depicts genomic loci matching the CHERV-3g and CHERV-1b RVLP sequences were identified by BLAST and/or RNA-sequencing, and verified by genomic PCR and sequencing. CHERV-3g was found to exist in the CHO genome assembly (GCF_003668045.3). Two alleles of CHERV-3g were found in CHO cells with minor differences in the two alleles. CHERV-1b was found to exist as one copy and not present in the CHO genome assembly. Part of the 3′LTR of CHERV-1b is truncated and a long telomere-like repeat is present, which is also present on the suspected 3′ flanking genomic sequence of the integration site. Guide RNAs directed to the CHO genome regions flanking these two loci were designed and the four sites were targeted simultaneously with CRISPR/Cas9 to stimulate large deletions. FIGS. 2B and 2C indicate that DL-8 and DL-17 showed reduced RVLP titers by RT-ddPCR and TEM. DL-11 and DL-19 showed reduced CHERV-3g titers but high CHERV-1b titers by RT-ddPCR. Accordingly, DL-19 showed a slight reduction in TEM titer. Yet DL-11 showed a strong reduction in TEM titer.

What is claimed is:

1. A modified Chinese Hamster Ovary (CHO) cell where, prior to the modification, the CHO cell comprises two or more endogenous retrovirus (ERV) loci selected from:
(a) CHERV-1b (Genbank Accession No. MN527960, SEQ ID NO. 8);
(b) CHERV-2g (Genbank Accession No. MN527961, SEQ ID NO. 9);
(c) ETC109F (30021-39247 of SEQ ID NO. 10);
(d) CHERV-3g (Genbank Accession No. MN527962, SEQ ID NO. 11); and
(e) an ERV locus comprising a sequence having at least 90% identity to any one of (a)-(d), and where the modification comprises inactivation of two or more of the ERV loci of (a)-(e).

2. The modified CHO cell of claim 1, wherein inactivation of the ERV loci comprises a knock-out of the respective ERV GAG coding sequence.

3. The modified CHO cell of claim 2, wherein the knock-out of each respective ERV GAG coding sequence comprises introduction of an indel into the ERV GAG coding sequence.

4. The modified CHO cell of claim 2, wherein the knock-out of each respective ERV GAG coding sequence comprises introduction of a base edit into the ERV GAG coding sequence.

5. The modified CHO cell of claim 2, wherein the knock-out of each respective ERV GAG coding sequence comprises introduction of a deletion flanking the ERV GAG coding sequence.

6. The modified CHO cell of claim 1, wherein the inactivation of one or more of the ERV loci comprises deletion of at least one ERV locus.

7. The modified CHO cell of claim 6, wherein the inactivation of one or more of the ERV loci comprises deletion of each ERV locus.

8. The modified CHO cell of claim 6, wherein the inactivation of one or more of the ERV loci comprises deletion of an ERV locus and introduction of an indel or a base edit into the ERV GAG coding sequence of an ERV locus.

9. The modified CHO cell of any one of claims 1-8, wherein the modified cell expresses a recombinant product of interest.

10. The modified CHO cell of any one of claims 1-8, wherein the modified cell is generated from a recombinant cell that expresses a recombinant product of interest.

11. The modified CHO cell of claim 9 or 10, wherein the recombinant product of interest comprises a recombinant protein.

12. The modified CHO cell of claim 11, wherein the recombinant protein is antibody or an antigen-binding fragment thereof.

13. The modified CHO cell of claim 12, wherein the antibody is a multispecific antibody or an antigen-binding fragment thereof.

14. The modified CHO cell of claim 13, wherein the antibody consists of a single heavy chain sequence and a single light chain sequence or antigen-binding fragments thereof.

15. The modified CHO cell of any one of claims 12-14, wherein the antibody is a chimeric antibody, a human antibody or a humanized antibody.

16. The modified CHO cell of any one of claims 12-15, wherein the antibody is a monoclonal antibody.

17. The modified CHO cell of claim 12, wherein the antibody is selected from: bevacizumab; trastuzumab; ranibizumab; efalizumab; rituximab, omalizumab, an anti-amyloid beta (Abeta) antibody; an anti-CD4 (MTRX1011A) antibody; an anti-EGFL7 (EGF-like-domain 7) antibody; an anti-IL13 antibody; an anti-Apomab antibody; an anti-DR5-targeted pro-apoptotic receptor agonist (PARA) antibody; an anti-BR3 antibody; an anti-CD268 antibody; an anti-BLyS receptor 3 antibody; an anti-BAFF-R (BAFF Receptor) antibody; an anti-beta 7 integrin subunit antibody; an anti-αᵥβ₈ integrin antibody; dacetuzumab (Anti-CD40); GA101 (anti-CD20 monoclonal antibody); MetMAb (anti-MET receptor tyrosine kinase antibody); an anti-neuropilin-1 (NRP1) antibody; ocrelizumab (anti-CD20 antibody); an anti-OX40 ligand antibody; an anti-oxidized LDL (oxLDL) antibody; pertuzumab (HER dimerization inhibitors (HDIs)); an anti-PD-L1 antibody; an anti-CD79b antibody; an anti-CD20×anti-CD3 bispecific antibody; an anti-VEGF-A×anti-angiopoietin-2 bispecific antibody; and rhuMAb IFN alpha.

18. The modified CHO cell of claim 10 or 11, wherein the recombinant product of interest is encoded by an exogenous nucleic acid sequence integrated in the cellular genome of the CHO cell at one or more targeted locations.

19. The modified CHO cell of claim 18, wherein the targeted location is at least about 90% homologous to a sequence of a portion of the contig sequence of one of the contigs NW_006874047.1, NW_006884592.1, NW_006881296.1, NW_003616412.1, NW_003615063.1, NW_006882936.1, and NW_003615411.1, or to a sequence selected from SEQ ID Nos. 1-7.

20. The modified CHO cell of claim 18, wherein the targeted location is within a sequence at least about 50% homologous to a 1000 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; or of NW_003615411.1 comprising position 97705.

21. The modified CHO cell of claim 19 or 20, wherein the modified CHO cell comprises gene knock-outs selected from: a) BAX; BAK; ICAM-1; GGTA1; CMAH; LPL; LPLA2; and PPT1; b) BAX; BAK; ICAM-1; SIRT-1; GGTA1; CMAH; LPL, LPLA2; and PPT1; c) BAX; BAK; ICAM-1; SIRT-1; MYC; GGTA1; CMAH; LPL; LPLA2; and PPT1; d) BAX; BAK; ICAM-1; SIRT-1; MYC; GGTA1; CMAH; LPL, LPLA2; PPT1; and LIPA; e) BAX; BAK; ICAM-1; GGTA1; CMAH; LPLA2; PPT1; and LIPA; f) BAX; BAK; ICAM-1; SIRT-1; GGTA1; CMAH; LPLA2; PPT1; and LIPA; g) BAX; BAK; ICAM-1; SIRT-1; MYC; GGTA1; CMAH; LPLA2; PPT1; and LIPA; h) BAX; BAK; ICAM-1; LPL; LPLA2; and PPT1; i) BAX; BAK; ICAM-1; SIRT-1; LPL, LPLA2; and PPT1; j) BAX; BAK; ICAM-1; SIRT-1; MYC; LPL; LPLA2; and PPT1; k) BAX; BAK; ICAM-1; SIRT-1; MYC; LPL; LPLA2; PPT1; and LIPA; 1) BAX; BAK; ICAM-1; SIRT-1; and MYC; m) BAX; BAK; ICAM-1; PERK; SIRT-1; and MYC; n) BAX; BAK; ICAM-1; SIRT-1; PERK; MYC; GGTA1; CMAH; LPL, LPLA2; PPT1; and LIPA; o) BAX; BAK; LPL; LPLA2; GGTA1; and CMAH; p) BAX; BAK; PERK; LPL; LPLA2; GGTA1; and CMAH; q) BAX; BAK; MYC; LPL; LPLA2; GGTA1; and CMAH; r) BAX; BAK; MYC; PERK; LPL; LPLA2; GGTA1; and CMAH; s) BAX; BAK; LPL; LPLA2; GGTA1; CMAH; and PPT1; t) BAX; BAK; PERK; LPL; LPLA2; GGTA1; CMAH; and PPT1; u) BAX; BAK; MYC; LPL; LPLA2; GGTA1; CMAH; and PPT1; v) BAX; BAK; MYC; PERK; LPL; LPLA2; GGTA1; CMAH; and PPT1; w) BAX; BAK; ICAM-1; and SIRT-1; x) BAX; BAK; and ICAM-1; y) BAX; BAK; BCKDHA; ICAM-1; GGTA1; CMAH; LPL; LPLA2; and PPT1; z) BAX; BAK; BCKDHA; ICAM-1; SIRT-1; GGTA1; CMAH; LPL; LPLA2; and PPT1; aa) BAX; BAK; BCKDHA; ICAM-1; SIRT-1; MYC; GGTA1; CMAH; LPL; LPLA2; and PPT1; bb) BAX; BAK; BCKDHA; ICAM-1; SIRT-1; MYC; GGTA1; CMAH; LPL, LPLA2; PPT1; and LIPA; cc) BAX; BAK; BCKDHA; ICAM-1; GGTA1; CMAH; LPLA2; PPT1; and LIPA; dd) BAX; BAK; BCKDHA; ICAM-1; SIRT-1; GGTA1; CMAH; LPLA2; PPT1; and LIPA; ee) BAX; BAK; BCKDHA; ICAM-1; SIRT-1; MYC; GGTA1; CMAH; LPLA2; PPT1; and LIPA; ff) BAX; BAK; BCKDHA; ICAM-1; LPL; LPLA2; and PPT1; gg) BAX; BAK; BCKDHA; ICAM-1; SIRT-1; LPL, LPLA2; and PPT1; hh) BAX; BAK; BCKDHA; ICAM-1; SIRT-1; MYC; LPL; LPLA2; and PPT1; ii) BAX; BAK; BCKDHA; ICAM-1; SIRT-1; MYC; LPL; LPLA2; PPT1; and LIPA; jj) BAX; BAK; BCKDHA; ICAM-1; SIRT-1; and MYC; kk) BAX; BAK; BCKDHA; ICAM-1; PERK; SIRT-1; and MYC; 11) BAX; BAK; BCKDHA; ICAM-1; PERK; SIRT-1; MYC; GGTA1; CMAH; LPL, LPLA2; PPT1; and LIPA; mm) BAX; BAK; BCKDHA; LPL; LPLA2; GGTA1; and CMAH; nn) BAX; BAK; BCKDHA; PERK; LPL; LPLA2; GGTA1; and CMAH; oo) BAX; BAK; BCKDHA; MYC; LPL; LPLA2; GGTA1; and CMAH; pp) BAX; BAK; BCKDHA; MYC; PERK; LPL; LPLA2; GGTA1; and CMAH; qq) BAX; BAK; BCKDHA; LPL; LPLA2; GGTA1; CMAH; and PPT1; rr) BAX; BAK; BCKDHA; PERK; LPL; LPLA2; GGTA1; CMAH; and PPT1; ss) BAX; BAK; BCKDHA; MYC; LPL; LPLA2; GGTA1; CMAH; and PPT1; tt) BAX; BAK; BCKDHA; MYC; PERK; LPL; LPLA2; GGTA1; CMAH; and PPT1; uu) BAX; BAK; BCKDHA; ICAM-1; and SIRT-1; vv) BAX; BAK; BCKDHA; and ICAM-1; ww) BAX; BAK; BCKDHB; ICAM-1; GGTA1; CMAH; LPL; LPLA2; and PPT1; xx) BAX; BAK; BCKDHB; ICAM-1; SIRT-1; GGTA1; CMAH; LPL; LPLA2; and PPT1; yy) BAX; BAK; BCKDHB; ICAM-1; SIRT-1; MYC; GGTA1; CMAH; LPL; LPLA2; and PPT1; zz) BAX; BAK; BCKDHB; ICAM-1; SIRT-1; MYC; GGTA1; CMAH; LPL, LPLA2; PPT1; and LIPA; aaa) BAX; BAK; BCKDHB; ICAM-1; GGTA1; CMAH; LPLA2; PPT1; and LIPA; bbb) BAX; BAK; BCKDHB; ICAM-1; SIRT-1; GGTA1; CMAH; LPLA2; PPT1; and LIPA; ccc) BAX; BAK; BCKDHB; ICAM-1; SIRT-1; MYC; GGTA1; CMAH; LPLA2; PPT1; and LIPA; ddd) BAX; BAK; BCKDHB; ICAM-1; LPL; LPLA2; and PPT1; eee) BAX; BAK; BCKDHB; ICAM-1; SIRT-1; LPL, LPLA2; and PPT1; fff) BAX; BAK; BCKDHB; ICAM-1; SIRT-1; MYC; LPL; LPLA2; and PPT1; ggg) BAX; BAK; BCKDHB; ICAM-1; SIRT-1; MYC; LPL; LPLA2; PPT1; and LIPA; hhh) BAX; BAK; BCKDHB; ICAM-1; SIRT-1; and MYC; iii) BAX; BAK; BCKDHB; ICAM-1; PERK; SIRT-1; and MYC; jjj) BAX; BAK; BCKDHB; ICAM-1; PERK; SIRT-1; MYC; GGTA1; CMAH; LPL, LPLA2; PPT1; and LIPA; kkk) BAX; BAK; BCKDHB; LPL; LPLA2; GGTA1; and CMAH; 111) BAX; BAK; BCKDHB; PERK; LPL; LPLA2; GGTA1; and CMAH; mmm) BAX; BAK; BCKDHB; MYC; LPL; LPLA2; GGTA1; and CMAH; nnn) BAX; BAK; BCKDHB; MYC; PERK; LPL; LPLA2; GGTA1; and CMAH; ooo) BAX; BAK; BCKDHB; LPL; LPLA2; GGTA1; CMAH; and PPT1; ppp) BAX; BAK; BCKDHB; PERK; LPL; LPLA2; GGTA1; CMAH; and PPT1; qqq) BAX; BAK; BCKDHB; MYC; LPL; LPLA2; GGTA1; CMAH; and PPT1; rrr) BAX; BAK; BCKDHB; MYC; PERK; LPL; LPLA2; GGTA1; CMAH; and PPT1; sss) BAX; BAK; BCKDHB; ICAM-1; and SIRT-1; ttt) BAX; BAK; BCKDHB; and ICAM-1; uuu) BAX; BAK; BCKDHA; BCKDHB; ICAM-1; GGTA1; CMAH; LPL; LPLA2; and PPT1; vvv) BAX; BAK; BCKDHA; BCKDHB; ICAM-1; SIRT-1; GGTA1; CMAH; LPL; LPLA2; and PPT1; www) BAX; BAK; BCKDHA; BCKDHB; ICAM-1; SIRT-1; MYC; GGTA1; CMAH; LPL; LPLA2; and PPT1; xxx) BAX; BAK; BCKDHA; BCKDHB; ICAM-1; SIRT-1; MYC; GGTA1; CMAH; LPL, LPLA2; PPT1; and LIPA; yyy) BAX; BAK; BCKDHA; BCKDHB; ICAM-1; GGTA1; CMAH; LPLA2; PPT1; and LIPA; zzz) BAX; BAK; BCKDHA; BCKDHB; ICAM-1; SIRT-1; GGTA1; CMAH; LPLA2; PPT1; and LIPA; aaaa) BAX; BAK; BCKDHA; BCKDHB; ICAM-1; SIRT-1; MYC; GGTA1; CMAH; LPLA2; PPT1; and LIPA; bbbb) BAX; BAK; BCKDHA; BCKDHB; ICAM-1; LPL; LPLA2; and PPT1; cccc) BAX; BAK; BCKDHA; BCKDHB; ICAM-1; SIRT-1; LPL, LPLA2; and PPT1; dddd) BAX; BAK; BCKDHA; BCKDHB; ICAM-1; SIRT-1; MYC; LPL; LPLA2; and PPT1; eeee) BAX; BAK; BCKDHA; BCKDHB; ICAM-1; SIRT-1; MYC; LPL; LPLA2; PPT1; and LIPA; ffff) BAX; BAK; BCKDHA; BCKDHB; ICAM-1; SIRT-1; and MYC; gggg) BAX; BAK; BCKDHA; BCKDHB; ICAM-1; PERK; SIRT-1; and MYC; hhhh) BAX; BAK; BCKDHA; BCKDHB; ICAM-1; PERK; SIRT-1; MYC; GGTA1; CMAH; LPL, LPLA2; PPT1; and LIPA; iiii) BAX; BAK; BCKDHA; BCKDHB; LPL; LPLA2; GGTA1; and CMAH; jjjj) BAX; BAK; BCKDHA; BCKDHB; PERK; LPL; LPLA2; GGTA1; and CMAH; kkkk) BAX; BAK; BCKDHA; BCKDHB; MYC; LPL; LPLA2; GGTA1; and CMAH; llll) BAX; BAK; BCKDHA; BCKDHB; MYC; PERK; LPL; LPLA2; GGTA1; and CMAH; mmmm) BAX; BAK; BCKDHA; BCKDHB; LPL; LPLA2; GGTA1; CMAH; and PPT1; nnnn) BAX; BAK; BCKDHA; BCKDHB; PERK; LPL; LPLA2; GGTA1; CMAH; and PPT1; oooo) BAX; BAK; BCKDHA; BCKDHB; MYC; LPL; LPLA2; GGTA1; CMAH; and PPT1; pppp) BAX; BAK; BCKDHA; BCKDHB; MYC; PERK; LPL; LPLA2; GGTA1; CMAH; and PPT1; qqqq) BAX; BAK; BCKDHA; BCKDHB; ICAM-1; and SIRT-1; or rrrr) BAX; BAK; BCKDHA; BCKDHB; and ICAM-1.

22. A method for producing a modified CHO cell, comprising:
(a) contacting the cell with a nuclease-assisted gene targeting system and/or nucleic acid targeting at least two endogenous ERVs selected from
(i) CHERV-1b (Genbank Accession No. MN527960, SEQ ID NO. 8);
(ii) CHERV-2g (Genbank Accession No. MN527961, SEQ ID NO. 9);
(iii) ETC109F (30021-39247 of SEQ ID NO. 10);
(iv) CHERV-3g (Genbank Accession No. MN527962, SEQ ID NO. 11); and
(v) an ERV locus comprising a sequence having at least 90% identity to any one of
(i)-(iv), and
(b) selecting the modified CHO cell wherein the expression of said ERVs has been reduced or eliminated as compared to an unmodified CHO cell.

23. The method according to claim 22, wherein an exogenous nucleic acid encoding a recombinant product of interest is introduced into the CHO cell after the modification of claim 21.

24. The method according to claim 23, wherein an exogenous nucleic acid encoding a recombinant product of interest is introduced into the CHO cell prior to the modification of claim 21.

25. The method of claim 23 or claim 24, wherein the exogenous nucleic acid encoding a recombinant product of interest is integrated in the cellular genome of the modified cells at one or more targeted locations.

26. The method of claim 25, wherein the targeted location is at least about 90% homologous to a sequence of a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7.

27. The method of claim 25, wherein the targeted location is within a sequence at least about 50% homologous to a 1000 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; or of NW_003615411.1 comprising position 97705.

28. The method of claim 23 or claim 24, wherein the exogenous nucleic acid encoding a recombinant product of interest is randomly integrated in the cellular genome of the modified CHO cells.

29. The method of any one of claims 22-28, wherein the recombinant product of interest comprises a recombinant protein.

30. The method of claim 29, wherein the recombinant protein is an antibody or an antigen-binding fragment thereof.

31. The method of claim 30, wherein antibody is a multispecific antibody or an antigen-binding fragment thereof.

32. The method of claim 31, wherein the antibody consists of a single heavy chain sequence and a single light chain sequence or antigen-binding fragments thereof.

33. The method of any one of claims 29-32, wherein the antibody is a chimeric antibody, a human antibody or a humanized antibody.

34. The method of any one of claims 29-33, wherein the antibody is a monoclonal antibody.

35. The method of claim 30, wherein the antibody is selected from: bevacizumab; trastuzumab; ranibizumab; efalizumab; rituximab, omalizumab, an anti-amyloid beta (Abeta) antibody; an anti-CD4 (MTRX1011A) antibody; an anti-EGFL7 (EGF-like-domain 7) antibody; an anti-IL13 antibody; an anti-Apomab antibody; an anti-DR5-targeted pro-apoptotic receptor agonist (PARA) antibody; an anti-BR3 antibody; an anti-CD268 antibody; an anti-BLyS receptor 3 antibody; an anti-BAFF-R (BAFF Receptor) antibody; an anti-beta 7 integrin subunit antibody; an anti-αᵥβ₈ integrin antibody; dacetuzumab (Anti-CD40); GA101 (anti-CD20 monoclonal antibody); MetMAb (anti-MET receptor tyrosine kinase antibody); an anti-neuropilin-1 (NRP1) antibody; ocrelizumab (anti-CD20 antibody); an anti-OX40 ligand antibody; an anti-oxidized LDL (oxLDL) antibody; pertuzumab (HER dimerization inhibitors (HDIs)); an anti-PD-L1 antibody; an anti-CD79b antibody; an anti-CD20×anti-CD3 bispecific antibody; an anti-VEGF-A×anti-angiopoietin-2 bispecific antibody; and rhuMAb IFN alpha.

36. The method of claim 23 or claim 24, wherein the nucleic acid sequence encoding the recombinant product of interest is integrated into the cellular genome of the CHO cell using a transposase-mediated gene integration system.

37. The method according to any one of claims 22-36 wherein the nuclease-assisted gene targeting system is selected from the group consisting of CRISPR/Cas9, CRISPR/Cpf1, zinc-finger nuclease, TALEN or meganuclease.

38. The method according to any one of claims 22-37, wherein the reduction of ERV expression is mediated by RNA silencing.

39. The method according to claim 38, wherein RNA silencing is selected from the group consisting of siRNA gene targeting and knock-down, shRNA gene targeting and knock-down, and miRNA gene targeting and knock-down.

40. The method according to any one of claims 22-39, wherein the modified CHO cell comprises gene knock-outs selected from: a) BAX; BAK; ICAM-1; GGTA1; CMAH; LPL; LPLA2; and PPT1; b) BAX; BAK; ICAM-1; SIRT-1; GGTA1; CMAH; LPL, LPLA2; and PPT1; c) BAX; BAK; ICAM-1; SIRT-1; MYC; GGTA1; CMAH; LPL; LPLA2; and PPT1; d) BAX; BAK; ICAM-1; SIRT-1; MYC; GGTA1; CMAH; LPL, LPLA2; PPT1; and LIPA; e) BAX; BAK; ICAM-1; GGTA1; CMAH; LPLA2; PPT1; and LIPA; f) BAX; BAK; ICAM-1; SIRT-1; GGTA1; CMAH; LPLA2; PPT1; and LIPA; g) BAX; BAK; ICAM-1; SIRT-1; MYC; GGTA1; CMAH; LPLA2; PPT1; and LIPA; h) BAX; BAK; ICAM-1; LPL; LPLA2; and PPT1; i) BAX; BAK; ICAM-1; SIRT-1; LPL, LPLA2; and PPT1; j) BAX; BAK; ICAM-1; SIRT-1; MYC; LPL; LPLA2; and PPT1; k) BAX; BAK; ICAM-1; SIRT-1; MYC; LPL; LPLA2; PPT1; and LIPA; 1) BAX; BAK; ICAM-1; SIRT-1; and MYC; m) BAX; BAK; ICAM-1; PERK; SIRT-1; and MYC; n) BAX; BAK; ICAM-1; SIRT-1; PERK; MYC; GGTA1; CMAH; LPL, LPLA2; PPT1; and LIPA; o) BAX; BAK; LPL; LPLA2; GGTA1; and CMAH; p) BAX; BAK; PERK; LPL; LPLA2; GGTA1; and CMAH; q) BAX; BAK; MYC; LPL; LPLA2; GGTA1; and CMAH; r) BAX; BAK; MYC; PERK; LPL; LPLA2; GGTA1; and CMAH; s) BAX; BAK; LPL; LPLA2; GGTA1; CMAH; and PPT1; t) BAX; BAK; PERK; LPL; LPLA2; GGTA1; CMAH; and PPT1; u) BAX; BAK; MYC; LPL; LPLA2; GGTA1; CMAH; and PPT1; v) BAX; BAK; MYC; PERK; LPL; LPLA2; GGTA1; CMAH; and PPT1; w) BAX; BAK; ICAM-1; and SIRT-1; x) BAX; BAK; and ICAM-1; y) BAX; BAK; BCKDHA; ICAM-1; GGTA1; CMAH; LPL; LPLA2; and PPT1; z) BAX; BAK; BCKDHA; ICAM-1; SIRT-1; GGTA1; CMAH; LPL; LPLA2; and PPT1; aa) BAX; BAK; BCKDHA; ICAM-1; SIRT-1; MYC; GGTA1; CMAH; LPL; LPLA2; and PPT1; bb) BAX; BAK; BCKDHA; ICAM-1; SIRT-1; MYC; GGTA1; CMAH; LPL, LPLA2; PPT1; and LIPA; cc) BAX; BAK; BCKDHA; ICAM-1; GGTA1; CMAH; LPLA2; PPT1; and LIPA; dd) BAX; BAK; BCKDHA; ICAM-1; SIRT-1; GGTA1; CMAH; LPLA2; PPT1; and LIPA; ee) BAX; BAK; BCKDHA; ICAM-1; SIRT-1; MYC; GGTA1; CMAH; LPLA2; PPT1; and LIPA; ff) BAX; BAK; BCKDHA; ICAM-1; LPL; LPLA2; and PPT1; gg) BAX; BAK; BCKDHA; ICAM-1; SIRT-1; LPL, LPLA2; and PPT1; hh) BAX; BAK; BCKDHA; ICAM-1; SIRT-1; MYC; LPL; LPLA2; and PPT1; ii) BAX; BAK; BCKDHA; ICAM-1; SIRT-1; MYC; LPL; LPLA2; PPT1; and LIPA; jj) BAX; BAK; BCKDHA; ICAM-1; SIRT-1; and MYC; kk) BAX; BAK; BCKDHA; ICAM-1; PERK; SIRT-1; and MYC; 11) BAX; BAK; BCKDHA; ICAM-1; PERK; SIRT-1; MYC; GGTA1; CMAH; LPL, LPLA2; PPT1; and LIPA; mm) BAX; BAK; BCKDHA; LPL; LPLA2; GGTA1; and CMAH; nn) BAX; BAK; BCKDHA; PERK; LPL; LPLA2; GGTA1; and CMAH; oo) BAX; BAK; BCKDHA; MYC; LPL; LPLA2; GGTA1; and CMAH; pp) BAX; BAK; BCKDHA; MYC; PERK; LPL; LPLA2; GGTA1; and CMAH; qq) BAX; BAK; BCKDHA; LPL; LPLA2; GGTA1; CMAH; and PPT1; rr) BAX; BAK; BCKDHA; PERK; LPL; LPLA2; GGTA1; CMAH; and PPT1; ss) BAX; BAK; BCKDHA; MYC; LPL; LPLA2; GGTA1; CMAH; and PPT1; tt) BAX; BAK; BCKDHA; MYC; PERK; LPL; LPLA2; GGTA1; CMAH; and PPT1; uu) BAX; BAK; BCKDHA; ICAM-1; and SIRT-1; vv) BAX; BAK; BCKDHA; and ICAM-1; ww) BAX; BAK; BCKDHB; ICAM-1; GGTA1; CMAH; LPL; LPLA2; and PPT1; xx) BAX; BAK; BCKDHB; ICAM-1; SIRT-1; GGTA1; CMAH; LPL; LPLA2; and PPT1; yy) BAX; BAK; BCKDHB; ICAM-1; SIRT-1; MYC; GGTA1; CMAH; LPL; LPLA2; and PPT1; zz) BAX; BAK; BCKDHB; ICAM-1; SIRT-1; MYC; GGTA1; CMAH; LPL, LPLA2; PPT1; and LIPA; aaa) BAX; BAK; BCKDHB; ICAM-1; GGTA1; CMAH; LPLA2; PPT1; and LIPA; bbb) BAX; BAK; BCKDHB; ICAM-1; SIRT-1; GGTA1; CMAH; LPLA2; PPT1; and LIPA; ccc) BAX; BAK; BCKDHB; ICAM-1; SIRT-1; MYC; GGTA1; CMAH; LPLA2; PPT1; and LIPA; ddd) BAX; BAK; BCKDHB; ICAM-1; LPL; LPLA2; and PPT1; eee) BAX; BAK; BCKDHB; ICAM-1; SIRT-1; LPL, LPLA2; and PPT1; fff) BAX; BAK; BCKDHB; ICAM-1; SIRT-1; MYC; LPL; LPLA2; and PPT1; ggg) BAX; BAK; BCKDHB; ICAM-1; SIRT-1; MYC; LPL; LPLA2; PPT1; and LIPA; hhh) BAX; BAK; BCKDHB; ICAM-1; SIRT-1; and MYC; iii) BAX; BAK; BCKDHB; ICAM-1; PERK; SIRT-1; and MYC; jjj) BAX; BAK; BCKDHB; ICAM-1; PERK; SIRT-1; MYC; GGTA1; CMAH; LPL, LPLA2; PPT1; and LIPA; kkk) BAX; BAK; BCKDHB; LPL; LPLA2; GGTA1; and CMAH; 111) BAX; BAK; BCKDHB; PERK; LPL; LPLA2; GGTA1; and CMAH; mmm) BAX; BAK; BCKDHB; MYC; LPL; LPLA2; GGTA1; and CMAH; nnn) BAX; BAK; BCKDHB; MYC; PERK; LPL; LPLA2; GGTA1; and CMAH; ooo) BAX; BAK; BCKDHB; LPL; LPLA2; GGTA1; CMAH; and PPT1; ppp) BAX; BAK; BCKDHB; PERK; LPL; LPLA2; GGTA1; CMAH; and PPT1; qqq) BAX; BAK; BCKDHB; MYC; LPL; LPLA2; GGTA1; CMAH; and PPT1; rrr) BAX; BAK; BCKDHB; MYC; PERK; LPL; LPLA2; GGTA1; CMAH; and PPT1; sss) BAX; BAK; BCKDHB; ICAM-1; and SIRT-1; ttt) BAX; BAK; BCKDHB; and ICAM-1; uuu) BAX; BAK; BCKDHA; BCKDHB; ICAM-1; GGTA1; CMAH; LPL; LPLA2; and PPT1; vvv) BAX; BAK; BCKDHA; BCKDHB; ICAM-1; SIRT-1; GGTA1; CMAH; LPL; LPLA2; and PPT1; www) BAX; BAK; BCKDHA; BCKDHB; ICAM-1; SIRT-1; MYC; GGTA1; CMAH; LPL; LPLA2; and PPT1; xxx) BAX; BAK; BCKDHA; BCKDHB; ICAM-1; SIRT-1; MYC; GGTA1; CMAH; LPL, LPLA2; PPT1; and LIPA; yyy) BAX; BAK; BCKDHA; BCKDHB; ICAM-1; GGTA1; CMAH; LPLA2; PPT1; and LIPA; zzz) BAX; BAK; BCKDHA; BCKDHB; ICAM-1; SIRT-1; GGTA1; CMAH; LPLA2; PPT1; and LIPA; aaaa) BAX; BAK; BCKDHA; BCKDHB; ICAM-1; SIRT-1; MYC; GGTA1; CMAH; LPLA2; PPT1; and LIPA; bbbb) BAX; BAK; BCKDHA; BCKDHB; ICAM-1; LPL; LPLA2; and PPT1; cccc) BAX; BAK; BCKDHA; BCKDHB; ICAM-1; SIRT-1; LPL, LPLA2; and PPT1; dddd) BAX; BAK; BCKDHA; BCKDHB; ICAM-1; SIRT-1; MYC; LPL; LPLA2; and PPT1; eeee) BAX; BAK; BCKDHA; BCKDHB; ICAM-1; SIRT-1; MYC; LPL; LPLA2; PPT1; and LIPA; ffff) BAX; BAK; BCKDHA; BCKDHB; ICAM-1; SIRT-1; and MYC; gggg) BAX; BAK; BCKDHA; BCKDHB; ICAM-1; PERK; SIRT-1; and MYC; hhhh) BAX; BAK; BCKDHA; BCKDHB; ICAM-1; PERK; SIRT-1; MYC; GGTA1; CMAH; LPL, LPLA2; PPT1; and LIPA; iiii) BAX; BAK; BCKDHA; BCKDHB; LPL; LPLA2; GGTA1; and CMAH; jjjj) BAX; BAK; BCKDHA; BCKDHB; PERK; LPL; LPLA2; GGTA1; and CMAH; kkkk) BAX; BAK; BCKDHA; BCKDHB; MYC; LPL; LPLA2; GGTA1; and CMAH; llll) BAX; BAK; BCKDHA; BCKDHB; MYC; PERK; LPL; LPLA2; GGTA1; and CMAH; mmmm) BAX; BAK; BCKDHA; BCKDHB; LPL; LPLA2; GGTA1; CMAH; and PPT1; nnnn) BAX; BAK; BCKDHA; BCKDHB; PERK; LPL; LPLA2; GGTA1; CMAH; and PPT1; oooo) BAX; BAK; BCKDHA; BCKDHB; MYC; LPL; LPLA2; GGTA1; CMAH; and PPT1; pppp) BAX; BAK; BCKDHA; BCKDHB; MYC; PERK; LPL; LPLA2; GGTA1; CMAH; and PPT1; qqqq) BAX; BAK; BCKDHA; BCKDHB; ICAM-1; and SIRT-1; or rrrr) BAX; BAK; BCKDHA; BCKDHB; and ICAM-1

41. A method for producing recombinant product of interest, comprising:
(a) culturing a CHO cell of any one of claims 9-21 under conditions resulting in the expression of the recombinant product of interest, and
(b) recovering the recombinant product of interest from the cultivation medium or the modified CHO cells.

42. The method of claim 41, wherein the exogenous nucleic acid encoding a recombinant product of interest is integrated in the cellular genome of the modified cells at one or more targeted locations.

43. The method of claim 42, wherein the targeted location is at least about 90% homologous to a sequence of a portion of the contig sequence of one of the contigs NW_006874047.1, NW_006884592.1, NW_006881296.1, NW_003616412.1, NW_003615063.1, NW_006882936.1, and NW_003615411.1 or to a sequence selected from SEQ ID Nos. 1-7.

44. The method of claim 42, wherein the targeted location is within a sequence at least about 50% homologous to a 1000 nucleotide sequence: of NW_006874047.1 comprising position 45269; of NW_006884592.1 comprising position 207911; of NW_006881296.1 comprising position 491909; of NW_003616412.1 comprising position 79768; of NW_003615063.1 comprising position 315265; of NW_006882936.1 comprising position 2662054; or of NW_003615411.1 comprising position 97705.

45. The method of claim 41, wherein the exogenous nucleic acid encoding a recombinant product of interest is randomly integrated in the cellular genome of the modified CHO cells.

46. The method of any one of claims 41-45, wherein the recombinant product of interest comprises a recombinant protein.

47. The method of claim 46, wherein the recombinant protein is an antibody or an antigen-binding fragment thereof.

48. The method of claim 47, wherein antibody is a multispecific antibody or an antigen-binding fragment thereof.

49. The method of claim 47, wherein the antibody consists of a single heavy chain sequence and a single light chain sequence or antigen-binding fragments thereof.

50. The method of any one of claims 47-49, wherein the antibody is a chimeric antibody, a human antibody or a humanized antibody.

51. The method of any one of claims 47-50, wherein the antibody is a monoclonal antibody.

52. The method of claim 47, wherein the antibody is selected from: bevacizumab; trastuzumab; ranibizumab; efalizumab; rituximab, omalizumab, an anti-amyloid beta (Abeta) antibody; an anti-CD4 (MTRX1011A) antibody; an anti-EGFL7 (EGF-like-domain 7) antibody; an anti-IL13 antibody; an anti-Apomab antibody; an anti-DR5-targeted pro-apoptotic receptor agonist (PARA) antibody; an anti-BR3 antibody; an anti-CD268 antibody; an anti-BLyS receptor 3 antibody; an anti-BAFF-R (BAFF Receptor) antibody; an anti-beta 7 integrin subunit antibody; an anti-αᵥβ₈ integrin antibody; dacetuzumab (Anti-CD40); GA101 (anti-CD20 monoclonal antibody); MetMAb (anti-MET receptor tyrosine kinase antibody); an anti-neuropilin-1 (NRP1) antibody; ocrelizumab (anti-CD20 antibody); an anti-OX40 ligand antibody; an anti-oxidized LDL (oxLDL) antibody; pertuzumab (HER dimerization inhibitors (HDIs)); an anti-PD-L1 antibody; an anti-CD79b antibody; an anti-CD20×anti-CD3 bispecific antibody; an anti-VEGF-A×anti-angiopoietin-2 bispecific antibody; and rhuMAb IFN alpha

53. The method of claim 41, wherein the nucleic acid sequence encoding the recombinant product of interest is integrated into the cellular genome of the CHO cell using a transposase-mediated gene integration system.

54. The method according to any one of claims 41-53 wherein the nuclease-assisted gene targeting system is selected from the group consisting of CRISPR/Cas9, CRISPR/Cpf1, zinc-finger nuclease, TALEN or meganuclease.

55. The method according to any one of claims 41-54, wherein the modified CHO cell comprises gene knock-outs selected from: a) BAX; BAK; ICAM-1; GGTA1; CMAH; LPL; LPLA2; and PPT1; b) BAX; BAK; ICAM-1; SIRT-1; GGTA1; CMAH; LPL, LPLA2; and PPT1; c) BAX; BAK; ICAM-1; SIRT-1; MYC; GGTA1; CMAH; LPL; LPLA2; and PPT1; d) BAX; BAK; ICAM-1; SIRT-1; MYC; GGTA1; CMAH; LPL, LPLA2; PPT1; and LIPA; e) BAX; BAK; ICAM-1; GGTA1; CMAH; LPLA2; PPT1; and LIPA; f) BAX; BAK; ICAM-1; SIRT-1; GGTA1; CMAH; LPLA2; PPT1; and LIPA; g) BAX; BAK; ICAM-1; SIRT-1; MYC; GGTA1; CMAH; LPLA2; PPT1; and LIPA; h) BAX; BAK; ICAM-1; LPL; LPLA2; and PPT1; i) BAX; BAK; ICAM-1; SIRT-1; LPL, LPLA2; and PPT1; j) BAX; BAK; ICAM-1; SIRT-1; MYC; LPL; LPLA2; and PPT1; k) BAX; BAK; ICAM-1; SIRT-1; MYC; LPL; LPLA2; PPT1; and LIPA; 1) BAX; BAK; ICAM-1; SIRT-1; and MYC; m) BAX; BAK; ICAM-1; PERK; SIRT-1; and MYC; n) BAX; BAK; ICAM-1; SIRT-1; PERK; MYC; GGTA1; CMAH; LPL, LPLA2; PPT1; and LIPA; o) BAX; BAK; LPL; LPLA2; GGTA1; and CMAH; p) BAX; BAK; PERK; LPL; LPLA2; GGTA1; and CMAH; q) BAX; BAK; MYC; LPL; LPLA2; GGTA1; and CMAH; r) BAX; BAK; MYC; PERK; LPL; LPLA2; GGTA1; and CMAH; s) BAX; BAK; LPL; LPLA2; GGTA1; CMAH; and PPT1; t) BAX; BAK; PERK; LPL; LPLA2; GGTA1; CMAH; and PPT1; u) BAX; BAK; MYC; LPL; LPLA2; GGTA1; CMAH; and PPT1; v) BAX; BAK; MYC; PERK; LPL; LPLA2; GGTA1; CMAH; and PPT1; w) BAX; BAK; ICAM-1; and SIRT-1; x) BAX; BAK; and ICAM-1; y) BAX; BAK; BCKDHA; ICAM-1; GGTA1; CMAH; LPL; LPLA2; and PPT1; z) BAX; BAK; BCKDHA; ICAM-1; SIRT-1; GGTA1; CMAH; LPL; LPLA2; and PPT1; aa) BAX; BAK; BCKDHA; ICAM-1; SIRT-1; MYC; GGTA1; CMAH; LPL; LPLA2; and PPT1; bb) BAX; BAK; BCKDHA; ICAM-1; SIRT-1; MYC; GGTA1; CMAH; LPL, LPLA2; PPT1; and LIPA; cc) BAX; BAK; BCKDHA; ICAM-1; GGTA1; CMAH; LPLA2; PPT1; and LIPA; dd) BAX; BAK; BCKDHA; ICAM-1; SIRT-1; GGTA1; CMAH; LPLA2; PPT1; and LIPA; ee) BAX; BAK; BCKDHA; ICAM-1; SIRT-1; MYC; GGTA1; CMAH; LPLA2; PPT1; and LIPA; ff) BAX; BAK; BCKDHA; ICAM-1; LPL; LPLA2; and PPT1; gg) BAX; BAK; BCKDHA; ICAM-1; SIRT-1; LPL, LPLA2; and PPT1; hh) BAX; BAK; BCKDHA; ICAM-1; SIRT-1; MYC; LPL; LPLA2; and PPT1; ii) BAX; BAK; BCKDHA; ICAM-1; SIRT-1; MYC; LPL; LPLA2; PPT1; and LIPA; jj) BAX; BAK; BCKDHA; ICAM-1; SIRT-1; and MYC; kk) BAX; BAK; BCKDHA; ICAM-1; PERK; SIRT-1; and MYC; 11) BAX; BAK; BCKDHA; ICAM-1; PERK; SIRT-1; MYC; GGTA1; CMAH; LPL, LPLA2; PPT1; and LIPA; mm) BAX; BAK; BCKDHA; LPL; LPLA2; GGTA1; and CMAH; nn) BAX; BAK; BCKDHA; PERK; LPL; LPLA2; GGTA1; and CMAH; oo) BAX; BAK; BCKDHA; MYC; LPL; LPLA2; GGTA1; and CMAH; pp) BAX; BAK; BCKDHA; MYC; PERK; LPL; LPLA2; GGTA1; and CMAH; qq) BAX; BAK; BCKDHA; LPL; LPLA2; GGTA1; CMAH; and PPT1; rr) BAX; BAK; BCKDHA; PERK; LPL; LPLA2; GGTA1; CMAH; and PPT1; ss) BAX; BAK; BCKDHA; MYC; LPL; LPLA2; GGTA1; CMAH; and PPT1; tt) BAX; BAK; BCKDHA; MYC; PERK; LPL; LPLA2; GGTA1; CMAH; and PPT1; uu) BAX; BAK; BCKDHA; ICAM-1; and SIRT-1; vv) BAX; BAK; BCKDHA; and ICAM-1; ww) BAX; BAK; BCKDHB; ICAM-1; GGTA1; CMAH; LPL; LPLA2; and PPT1; xx) BAX; BAK; BCKDHB; ICAM-1; SIRT-1; GGTA1; CMAH; LPL; LPLA2; and PPT1; yy) BAX; BAK; BCKDHB; ICAM-1; SIRT-1; MYC; GGTA1; CMAH; LPL; LPLA2; and PPT1; zz) BAX; BAK; BCKDHB; ICAM-1; SIRT-1; MYC; GGTA1; CMAH; LPL, LPLA2; PPT1; and LIPA; aaa) BAX; BAK; BCKDHB; ICAM-1; GGTA1; CMAH; LPLA2; PPT1; and LIPA; bbb) BAX; BAK; BCKDHB; ICAM-1; SIRT-1; GGTA1; CMAH; LPLA2; PPT1; and LIPA; ccc) BAX; BAK; BCKDHB; ICAM-1; SIRT-1; MYC; GGTA1; CMAH; LPLA2; PPT1; and LIPA; ddd) BAX; BAK; BCKDHB; ICAM-1; LPL; LPLA2; and PPT1; eee) BAX; BAK; BCKDHB; ICAM-1; SIRT-1; LPL, LPLA2; and PPT1; fff) BAX; BAK; BCKDHB; ICAM-1; SIRT-1; MYC; LPL; LPLA2; and PPT1; ggg) BAX; BAK; BCKDHB; ICAM-1; SIRT-1; MYC; LPL; LPLA2; PPT1; and LIPA; hhh) BAX; BAK; BCKDHB; ICAM-1; SIRT-1; and MYC; iii) BAX; BAK; BCKDHB; ICAM-1; PERK; SIRT-1; and MYC; jjj) BAX; BAK; BCKDHB; ICAM-1; PERK; SIRT-1; MYC; GGTA1; CMAH; LPL, LPLA2; PPT1; and LIPA; kkk) BAX; BAK; BCKDHB; LPL; LPLA2; GGTA1; and CMAH; 111) BAX; BAK; BCKDHB; PERK; LPL; LPLA2; GGTA1; and CMAH; mmm) BAX; BAK; BCKDHB; MYC; LPL; LPLA2; GGTA1; and CMAH; nnn) BAX; BAK; BCKDHB; MYC; PERK; LPL; LPLA2; GGTA1; and CMAH; ooo) BAX; BAK; BCKDHB; LPL; LPLA2; GGTA1; CMAH; and PPT1; ppp) BAX; BAK; BCKDHB; PERK; LPL; LPLA2; GGTA1; CMAH; and PPT1; qqq) BAX; BAK; BCKDHB; MYC; LPL; LPLA2; GGTA1; CMAH; and PPT1; rrr) BAX; BAK; BCKDHB; MYC; PERK; LPL; LPLA2; GGTA1; CMAH; and PPT1; sss) BAX; BAK; BCKDHB; ICAM-1; and SIRT-1; ttt) BAX; BAK; BCKDHB; and ICAM-1; uuu) BAX; BAK; BCKDHA; BCKDHB; ICAM-1; GGTA1; CMAH; LPL; LPLA2; and PPT1; vvv) BAX; BAK; BCKDHA; BCKDHB; ICAM-1; SIRT-1; GGTA1; CMAH; LPL; LPLA2; and PPT1; www) BAX; BAK; BCKDHA; BCKDHB; ICAM-1; SIRT-1; MYC; GGTA1; CMAH; LPL; LPLA2; and PPT1; xxx) BAX; BAK; BCKDHA; BCKDHB; ICAM-1; SIRT-1; MYC; GGTA1; CMAH; LPL, LPLA2; PPT1; and LIPA; yyy) BAX; BAK; BCKDHA; BCKDHB; ICAM-1; GGTA1; CMAH; LPLA2; PPT1; and LIPA; zzz) BAX; BAK; BCKDHA; BCKDHB; ICAM-1; SIRT-1; GGTA1; CMAH; LPLA2; PPT1; and LIPA; aaaa) BAX; BAK; BCKDHA; BCKDHB; ICAM-1; SIRT-1; MYC; GGTA1; CMAH; LPLA2; PPT1; and LIPA; bbbb) BAX; BAK; BCKDHA; BCKDHB; ICAM-1; LPL; LPLA2; and PPT1; cccc) BAX; BAK; BCKDHA; BCKDHB; ICAM-1; SIRT-1; LPL, LPLA2; and PPT1; dddd) BAX; BAK; BCKDHA; BCKDHB; ICAM-1; SIRT-1; MYC; LPL; LPLA2; and PPT1; eeee) BAX; BAK; BCKDHA; BCKDHB; ICAM-1; SIRT-1; MYC; LPL; LPLA2; PPT1; and LIPA; ffff) BAX; BAK; BCKDHA; BCKDHB; ICAM-1; SIRT-1; and MYC; gggg) BAX; BAK; BCKDHA; BCKDHB; ICAM-1; PERK; SIRT-1; and MYC; hhhh) BAX; BAK; BCKDHA; BCKDHB; ICAM-1; PERK; SIRT-1; MYC; GGTA1; CMAH; LPL, LPLA2; PPT1; and LIPA; iiii) BAX; BAK; BCKDHA; BCKDHB; LPL; LPLA2; GGTA1; and CMAH; jjjj) BAX; BAK; BCKDHA; BCKDHB; PERK; LPL; LPLA2; GGTA1; and CMAH; kkkk) BAX; BAK; BCKDHA; BCKDHB; MYC; LPL; LPLA2; GGTA1; and CMAH; llll) BAX; BAK; BCKDHA; BCKDHB; MYC; PERK; LPL; LPLA2; GGTA1; and CMAH; mmmm) BAX; BAK; BCKDHA; BCKDHB; LPL; LPLA2; GGTA1; CMAH; and PPT1; nnnn) BAX; BAK; BCKDHA; BCKDHB; PERK; LPL; LPLA2; GGTA1; CMAH; and PPT1; oooo) BAX; BAK; BCKDHA; BCKDHB; MYC; LPL; LPLA2; GGTA1; CMAH; and PPT1; pppp) BAX; BAK; BCKDHA; BCKDHB; MYC; PERK; LPL; LPLA2; GGTA1; CMAH; and PPT1; qqqq) BAX; BAK; BCKDHA; BCKDHB; ICAM-1; and SIRT-1; or rrrr) BAX; BAK; BCKDHA; BCKDHB; and ICAM-1

56. The method of any of claims 41-55, comprising purifying the recombinant product of interest, and/or formulating the recombinant product of interest.
